(19) United States
(12) Patent Application Publication
(10) Pub. No.: US 2011/0288116 A1
(43) Pub. Date: Nov. 24, 2011

(54) IAP INHIBITORS

(75) Inventors: Stephen M. Condon, Malvern, PA (US); Matthew G. Laporte, Malvern, PA (US); Yijun Deng, Malvern, PA (US); Susan Rippin, Malvern, PA (US); Yu-Hua Lee, Malvern, PA (US); Thomas Haimowitz, Malvern, PA (US)

(73) Assignee: TETRALOGIC PHARMACEUITCAL CORPORATION, Malvern, PA (US)

(21) Appl. No.: 12/863,375

(22) PCT Filed: Jan. 15, 2009
(86) PCT No.: PCT/US2009/031093

§ 371 Date: Aug. 31, 2010

Related U.S. Application Data
(60) Provisional application No. 61/023,237, filed on Jan. 24, 2008.

(51) Int. Cl. A61K 31/4045; C12N 5/09; C07D 403/06; A61P 17/06; A61P 35/00; A61P 35/02; A61P 37/00; C07D 471/04; A61K 31/437

(52) U.S. Cl. 514/300; 546/113; 435/375; 548/468; 514/414

(57) ABSTRACT

The present invention describes compounds of the following formula: processes for their preparation, pharmaceutical compositions containing them, and their use in therapy. The compounds of the present invention inhibit IAPs (inhibitors of apoptosis proteins) and thus are useful in the treatment of cancer, autoimmune diseases and other disorders where a defect in apoptosis is implicated.

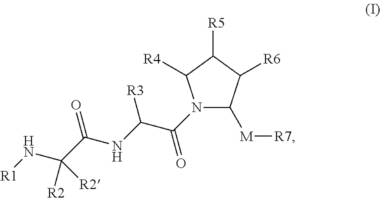

### BACKGROUND OF THE INVENTION

1. Field of the Invention

The present invention describes compounds that inhibit IAPs (inhibitors of apoptosis proteins), processes for their preparation, pharmaceutical compositions containing them, and their use in therapy. The compounds of the present invention are useful in the treatment of cancer, autoimmune diseases and other disorders where a defect in apoptosis is implicated.

2. Description of Related Art

Apoptosis (programmed cell death) plays a central role in the development and homeostasis of all multi-cellular organisms. Apoptosis can be initiated within a cell from an external factor such as a chemokine (an extrinsic pathway) or via an intracellular event such a DNA damage (an intrinsic pathway). Alterations in apoptotic pathways have been implicated in many types of human pathologies, including developmental disorders, cancer, autoimmune diseases, as well as neuro-degenerative disorders. One mode of action of chemotherapeutic drugs is cell death via apoptosis.

Apoptosis is conserved across species and executed primarily by activated caspases, a family of cysteine proteases with aspartate specificity in their substrates. These cysteine containing aspartate specific proteases (“caspases”) are produced in cells as catalytically inactive zymogens and are proteolytically processed to become active proteases during apoptosis. Once activated, effector caspases are responsible for proteolytic cleavage of a broad spectrum of cellular targets that ultimately lead to cell death. In normal surviving cells that have not received an apoptotic stimulus, most caspases remain inactive. If caspases are aberrantly activated, their proteolytic activity can be inhibited by a family of evolutionarily conserved proteins called IAPs (inhibitors of apoptosis proteins).

The IAP family of proteins suppresses apoptosis by preventing the activation of procaspases and inhibiting the enzymatic activity of mature caspases. Several distinct mammalian IAPs including XIAP, c-IAP1, c-IAP2, ML-IAP, NAIP (neuronal apoptosis inhibiting protein), Bruce, and survivin, have been identified, and they all exhibit anti-apoptotic activity in cell culture. IAPs were originally discovered in baculovirus by their functional ability to substitute for P35 protein, an anti-apoptotic gene. IAPs have been described in organisms ranging from Drosophila to human, and are known to be overexpressed in many human cancers. Generally speaking, IAPs comprise one to three Baculovirus IAP repeat (BIR) domains, and most of them also possess a carboxyl-terminal RING finger motif. The BIR domain itself is a zinc binding domain of about 70 residues comprising 4 alpha-helices and 3 beta strands, with cysteine and histidine residues that coordinate the zinc ion. It is the BIR domain that is believed to cause the anti-apoptotic effect by inhibiting the caspases and thus inhibiting apoptosis. XIAP is expressed ubiquitously in most adult and fetal tissues. Overexpression of XIAP in tumor cells has been demonstrated to confer protection against a variety of pro-apoptotic stimuli and promotes resistance to chemotherapy. Consistent with this, a strong correlation between XIAP protein levels and survival has been demonstrated for patients with acute myelogenous leukemia. Down-regulation of XIAP expression by antisense oligonucleotides has been shown to sensitize tumor cells to death induced by a wide range of pro-apoptotic agents, both in vitro and in vivo

In normal cells signaled to undergo apoptosis, however, the IAP-mediated inhibitory effect must be removed, a process at least in part performed by a mitochondrial protein named Smac (second mitochondrial activator of caspases). Smac (or, DIABLO), is synthesized as a precursor molecule of 239 amino acids; the N-terminal 55 residues serve as the mitochondria targeting sequence that is removed after import. The mature form of Smac contains 184 amino acids and behaves as an oligomer in solution. Smac and various fragments thereof have been proposed for use as targets for identification of therapeutic agents.

Smac is synthesized in the cytoplasm with an N-terminal mitochondrial targeting sequence that is proteolytically removed during maturation to the mature polypeptide and is then targeted to the inter-membrane space of mitochondria. At the time of apoptosis induction, Smac is released from mitochondria into the cytosol, together with cytochrome c, where it binds to IAPs, and enables caspase activation, therein eliminating the inhibitory effect of IAPs on apoptosis. Whereas cytochrome c induces multimerization of Apaf-1 to activate procaspase-9 and -3, Smac eliminates the inhibitory effect of multiple IAPs. Smac interacts with essentially all IAPs that have been examined to date including XIAP, c-IAP1, c-IAP2, ML-IAP, and survivin. Thus, Smac appears to be a master regulator of apoptosis in mammals.

It has been shown that Smac promotes not only the proteolytic activation of procaspases, but also the enzymatic activity of mature caspase, both of which depend upon its ability to interact physically with IAPs. X-ray crystallography has shown that the first four amino acids (AVPI) of mature Smac bind to a portion of IAPs. This N-terminal sequence is essential for binding IAPs and blocking their anti-apoptotic effects.

Current trends in cancer drug design focus on selective targeting to activate the apoptotic signaling pathways within tumors while sparing normal cells. The tumor specific properties of specific chemotherapeutic agents, such as TRAIL (tumor necrosis factor-related apoptosis-inducing ligand) have been reported. TRAIL is one of several members of the tumor necrosis factor (TNF) superfamily that induce apoptosis through the engagement of death receptors. TRAIL interacts with an unusually complex receptor system, which in humans comprises two death receptors and three decoy receptors. TRAIL has been used as an anti-cancer agent alone and in combination with other agents including ionizing radiation. TRAIL can initiate apoptosis in cells that overexpress the survival factors Bcl-2 and Bcl-XL, and may represent a treatment strategy for tumors that have acquired resistance to chemotherapeutic drugs. TRAIL binds its cognate receptors and activates the caspase cascade utilizing adapter molecules such as TRADD (TNF Receptor-Associated Death Domain). TRAIL signaling can be inhibited by overexpression of cIAP-1 or 2, indicating an important role for these proteins in the signaling pathway. Currently, five TRAIL receptors have been identified. Two receptors TRAIL-R1 (DR4) and TRAIL-R2 (DR5) mediate apoptotic signaling, and three non-functional receptors, DcR1, DcR2, and osteoprotegerin (OPG) may act as decoy receptors. Agents that increase expression of DR4 and DR5 may exhibit synergistic anti-tumor activity when combined with TRAIL.

Currently, there are drug discovery efforts aimed at identifying compounds that interfere with the role played by IAPs in disease states where a defect in apoptosis is implicated, such as in cancers and autoimmune diseases.

### SUMMARY OF THE INVENTION

The present invention provides IAP inhibitors and therapeutic methods of using these inhibitors to modulate apoptosis.

In one aspect the present invention provides compound of Formula (I):

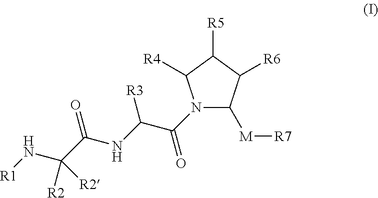

or a pharmaceutically acceptable salt thereof,

wherein:

R1 is H, hydroxy, alkyl, alkenyl, cycloalkyl, heterocycloalkyl, aryl, alkoxy, aryloxy, or heteroaryl;

R2 and R2′ are each independently H, alkyl, cycloalkyl, or heterocycloalkyl; or when R2′ is H then R2 and R1 can together form an aziridine or azetidine ring;

R3 and R4 are each independently H, alkyl, cycloalkyl, heterocycloalkyl, aryl, or heteroaryl; or, R3 and R4 are both carbon atoms linked by a covalent bond or by an alkylene or alkenylene group of 1 to 8 carbon atoms where one to three carbon atoms can be replaced by O, S(O)ₙ or N(R8);

R5 is H, hydroxy, alkoxy, aryloxy, alkyl, cycloalkyl, heterocycloalkyl, aryl, or heteroaryl;

R6 is H, hydroxy, alkoxy, aryloxy, alkyl, cycloalkyl, heterocycloalkyl, aryl, or heteroaryl,

R7 is alkyl, cycloalkyl, heterocycloalkyl, aryl, or heteroaryl;

R8 is H, hydroxy, alkoxy, aryloxy, alkyl, cycloalkyl, heterocycloalkyl, aryl, or heteroaryl;

M is a bond or an alkylene group of 1 to 5 carbon atoms;

n is 1 or 2, and

subject to the proviso that when R5 and R6 are both H, or when R5 is aryloxy and R6 is H, then either (1) R3 and R4 are both carbon atoms linked by a covalent bond or by an alkylene or alkenylene group of 1 to 8 carbon atoms where one to three carbon atoms can be replaced by O, S(O)ₙ, or N(R8), or (2) R7 is selected from

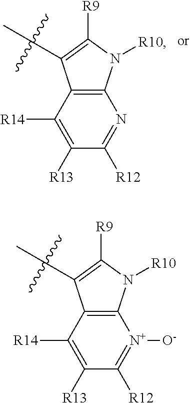

where R9, R10, R12, R13 and R14 are independently selected from hydroxy, alkoxy, aryloxy, alkyl, or aryl.

In another aspect, the present invention provides compounds of Formula (II):

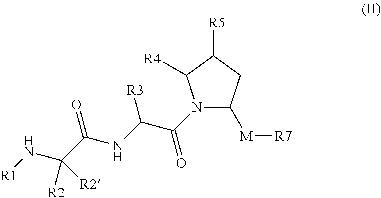

or a pharmaceutically acceptable salt thereof,

wherein:

R1 is H, hydroxy, alkyl, alkenyl, cycloalkyl, heterocycloalkyl, aryl, alkoxy, aryloxy, or heteroaryl;

R2 and R2′ are each independently H, alkyl, cycloalkyl, or heterocycloalkyl; or when R2′ is H then R2 and R1 can together form an aziridine or anticline ring;

R3 and R4 are each independently H, alkyl, cycloalkyl, heterocycloalkyl, aryl, or heteroaryl; or, R3 and R4 are both carbon atoms linked by a covalent bond or by an alkylene or alkenylene group of 1 to 8 carbon atoms where one to three carbon atoms can be replaced by O, S(O)ₙ or N(R8);

R5 is H, hydroxy, alkoxy, aryloxy, alkyl, cycloalkyl, heterocycloalkyl, aryl, or heteroaryl;

R7 is alkyl, cycloalkyl, heterocycloalkyl, aryl, or heteroaryl;

R8 is H, hydroxy, alkoxy, aryloxy, alkyl, cycloalkyl, heterocycloalkyl, aryl, or heteroaryl;

M is a bond or an alkylene group of 1 to 5 carbon atoms;

n is 1 or 2, and

subject to the proviso that when R5 is H, or aryloxy, then either (1) R3 and R4 are both carbon atoms linked by a covalent bond or by an alkylene or alkenylene group of 1 to 8 carbon atoms where one to three carbon atoms can be replaced by O, S(O)ₙ or N(R8), or (2) R7 is selected from

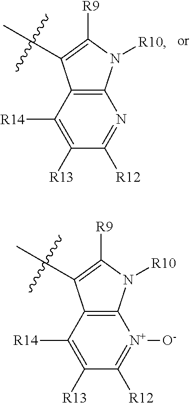

where R9, R10, R12, R13 and R14 are independently selected from hydroxy, alkoxy, aryloxy, alkyl, or aryl.

In yet another aspect, the present invention provides compounds of formula (IV)

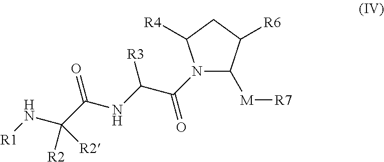

or a pharmaceutically acceptable salt thereof,

wherein:

R1 is H, hydroxy, alkyl, alkenyl, cycloalkyl, heterocycloalkyl, aryl, alkoxy, aryloxy, or heteroaryl;

R2 and R2′ are each independently H, alkyl, cycloalkyl, or heterocycloalkyl; or when R2′ is H then R2 and R1 can together form an aziridine or azetidine;

R3 and R4 are each independently H, alkyl, cycloalkyl, heterocycloalkyl, aryl, or heteroaryl; or, R3 and R4 are both carbon atoms linked by a covalent bond or by an alkylene or alkenylene group of 1 to 8 carbon atoms where one to three carbon atoms can be replaced by O, S(O)ₙ or N(R8),

R6 is hydroxy, alkoxy, aryloxy, alkyl, cycloalkyl, heterocycloalkyl, aryl, or heteroaryl;

R7 is alkyl, cycloalkyl, heterocycloalkyl, aryl, or heteroaryl;

R8 is H, hydroxy, alkoxy, aryloxy, alkyl, cycloalkyl, heterocycloalkyl, aryl, or heteroaryl;

M is a bond or an alkylene group of 1 to 5 carbon atoms; and

n is 1 or 2.

For simplicity and illustrative purposes, the principles of the invention are described by referring mainly to specific illustrative embodiments thereof. In addition, in the following description, numerous specific details are set forth in order to provide a thorough understanding of the invention. It will be apparent however, to one of ordinary skill in the art, that the invention may be practiced without limitation to these specific details. In other instances, well known methods and structures have not been described in detail so as not to unnecessarily obscure the invention.

### DEFINITIONS

“Alkyl” (monovalent) and “alkylene” (divalent) when alone or as part of another term (e.g., alkoxy) mean a branched or unbranched, saturated aliphatic hydrocarbon group, having up to 12 carbon atoms unless otherwise specified. Examples of particular alkyl groups include, but are not limited to, methyl, ethyl, n-propyl, isopropyl, n-butyl, iso-butyl, sec-butyl, tert-butyl, n-pentyl, 2-methylbutyl, 2,2-dimethylpropyl, n-hexyl, 2-methylpentyl, 2,2-dimethylbutyl, n-heptyl, 3-heptyl, 2-methylhexyl, and the like. The terms “lower alkyl”, “C₁-C₄ alkyl” and “alkyl of 1 to 4 carbon atoms” are synonymous and used interchangeably to mean methyl, ethyl, 1-propyl, isopropyl, cyclopropyl, 1-butyl, sec-butyl or t-butyl. Examples of alkylene groups include, but are not limited to, methylene, ethylene, n-propylene, n-butylene and 2-methyl-butylene. The term alkyl includes both “unsubstituted alkyls” and “substituted alkyls,” (unless the context clearly indicates otherwise) the latter of which refers to alkyl moieties having substituents replacing one or more hydrogens on one or more (often no more than four) carbon atoms of the hydrocarbon backbone. Such substituents are independently selected from the group consisting of halo (e.g., I, Br, Cl, F), hydroxy, alkenyl, alkynyl, amino, cyano, alkoxy (such as C₁-C₆ alkoxy), aryloxy (such as phenoxy), nitro, carboxyl, oxo, carbamoyl, cycloalkyl, aryl (e.g., aralkyls or arylalkyls), heterocyclyl, heteroaryl, alkylsulfonyl, arylsulfonyl and —OCF₃. Exemplary substituted alkyl groups include cyanomethyl, nitromethyl, hydroxymethyl, trityloxymethyl, propionyloxymethyl, aminomethyl, carboxymethyl, carboxyethyl, carboxypropyl, 2,3-dichloropentyl, 3-hydroxy-5-carboxyhexyl, acetyl (where the two hydrogen atoms on the —CH₂ portion of an ethyl group are replaced by an oxo (═O), 2-aminopropyl, pentachlorobutyl, trifluoromethyl, methoxyethyl, 3-hydroxypentyl, 4-chlorobutyl, 1,2-dimethyl-propyl, pentafluoroethyl, alkyloxycarbonylmethyl, allyloxycarbonylaminomethyl, carbamoyloxymethyl, methoxymethyl, ethoxymethyl, t-butoxymethyl, acetoxymethyl, chloromethyl, bromomethyl, iodomethyl, trifluoromethyl, 6-hydroxyhexyl, 2,4-dichloro (n-butyl), 2-amino (iso-propyl), and 2-carbamoyloxyethyl. Particular substituted alkyls are substituted methyl groups. Examples of substituted methyl group include groups such as hydroxymethyl, protected hydroxymethyl (e.g., tetrahydropyranyl-oxymethyl), acetoxymethyl, carbamoyloxymethyl, trifluoromethyl, chloromethyl, carboxymethyl, carboxyl (where the three hydrogen atoms on the methyl are replaced, two hydrogens are replaced by an oxo (═O) and the other hydrogen is replaced by a hydroxy (—OH), bromomethyl and iodomethyl. The term alkylene includes both “unsubstituted alkylenes” and “substituted alkylenes,” (unless the context clearly indicates otherwise). The alkylene groups can be similarly be substituted with groups as set forth above for alkyl.

“Alkenyl” (monovalent) and “alkenylene” (divalent) when alone or as part of another term mean a unsaturated hydrocarbon group containing at least one carbon-carbon double bond, typically 1 or 2 carbon-carbon double bonds, and which may be linear or branched. Representative alkenyl groups include, by way of example, vinyl, allyl, isopropenyl, but-2-enyl, n-pent-2-enyl, and n-hex-2-enyl. The terms alkenyl and alkenylene include both “unsubstituted alkenyls” and “substituted alkenyls,” as well as both “unsubstituted alkenylenes” and “substituted alkenylenes,” (unless the context clearly indicates otherwise). The substituted versions refer to alkenyl and alkenylene moieties having substituents replacing one or more hydrogens on one or more (often no more than four) carbon atoms of the hydrocarbon backbone. Such substituents are independently selected from the group consisting of: halo (e.g., I, Br, Cl, F), hydroxy, amino, cyano, alkoxy (such as C₁-C₆ alkoxy), aryloxy (such as phenoxy), nitro, carboxyl, oxo, carbamoyl, cycloalkyl, aryl (e.g., aralkyls), heterocyclyl, heteroaryl, alkylsulfonyl, arylsulfonyl and —OCF₃.

“Alkynyl” means a monovalent unsaturated hydrocarbon group containing at least one carbon-carbon triple bond, typically 1 carbon-carbon triple bond, and which may be linear or branched. Representative alkynyl groups include, by way of example, ethynyl, propargyl, and but-2-ynyl.

“Cycloalkyl” when alone or as part of another term means a saturated or partially unsaturated cyclic aliphatic hydrocarbon group (carbocycle group), having up to 12 carbon atoms unless otherwise specified and includes cyclic and polycyclic, including fused cycloalkyl. The term cycloalkyl includes both “unsubstituted cycloalkyls” and “substituted cycloalkyls,” (unless the context clearly indicates otherwise) the latter of which refers to cycloalkyl moieties having substituents replacing one or more hydrogens on one or more (often no more than four) carbon atoms of the hydrocarbon backbone. Such substituents are independently selected from the group consisting of: halo (e.g., I, Br, Cl, F), hydroxy, amino, cyano, alkoxy (such as C₁-C₆ alkoxy), aryloxy (such as phenoxy), nitro, carboxyl, oxo, carbamoyl, alkyl (including substituted alkyls such as trifluoromethyl), aryl, heterocyclyl, heteroaryl, alkylsulfonyl, arylsulfonyl and —OCF₃. Examples of cycloalkyls include cyclopropy, cyclobutyl, cyclopentyl, cyclohexyl, tetrahydronaphthyl and indanyl.

“Amino” denotes primary (i.e., —NH₂), secondary (i.e., —NHR) and tertiary (i.e., —NRR) amines, where the R groups can be a variety of moieties, usually an alkyl or an aryl. Particular secondary and tertiary amines are alkylamines, dialkylamines, arylamines, diarylamines, aralkylamines and diaralkylamines. Particular secondary and tertiary amines are methylamine, ethylamine, propylamine, isopropylamine, phenylamine, benzylamine dimethylamine, diethylamine, dipropylamine and disopropylamine.

“Aryl” when used alone or as part of another term means an aromatic carbocyclic group whether or not fused having the number of carbon atoms designated or if no number is designated, from 6 up to 14 carbon atoms. Particular aryl groups include phenyl, naphthyl, biphenyl, phenanthrenyl, naphthacenyl, and the like (see e.g. Lang's Handbook of Chemistry (Dean, J. A., ed) 13^{th} ed. Table 7-2 [1985]). Phenyl groups are generally preferred. The term aryl includes both “unsubstituted aryls” and “substituted aryls” (unless the context clearly indicates otherwise), the latter of which refers to aryl moieties having substituents replacing one or more hydrogens on one or more (usually no more than six) carbon atoms of the hydrocarbon backbone. Such substituents are independently selected from the group consisting of: halo (e.g., I, Br, Cl, F), hydroxy, amino, cyano, alkoxy (such as C₁-C₆ alkoxy), aryloxy (such as phenoxy), nitro, carboxyl, oxo, carbamoyl, alkyl (such as trifluoromethyl), aryl, —OCF₃, alkylsulfonyl, arylsulfonyl, heterocyclyl and heteroaryl. Examples of such substituted phenyls include but are not limited to a mono- or di (halo) phenyl group such as 2-chlorophenyl, 2-bromophenyl, 4-chlorophenyl, 2,6-dichlorophenyl, 2,5-dichlorophenyl, 3,4-dichlorophenyl, 3-chlorophenyl, 3-bromophenyl, 4-bromophenyl, 3,4-dibromophenyl, 3-chloro-4-fluorophenyl, 2-fluorophenyl; a mono- or di (hydroxy)phenyl group such as 4-hydroxyphenyl, 3-hydroxyphenyl, 2,4-dihydroxyphenyl, the protected-hydroxy derivatives thereof; a nitrophenyl group such as 3- or 4-nitrophenyl; a cyanophenyl group, for example, 4-cyanophenyl; a mono- or di (lower alkyl)phenyl group such as 4-methylphenyl, 2,4-dimethylphenyl, 2-methylphenyl, 4-(iso-propyl)phenyl, 4-ethylphenyl, 3-(n-propyl)phenyl; a mono or di (alkoxy)phenyl group, for example, 3,4-dimethoxyphenyl, 3-methoxy-4-benzyloxyphenyl, 3-methoxy-4-(1-chloromethyl)benzyloxy-phenyl, 3-ethoxyphenyl, 4-(isopropoxy)phenyl, 4-(t-butoxy)phenyl, 3-ethoxy-4-methoxyphenyl; 3- or 4-trifluoromethylphenyl; a mono- or dicarboxyphenyl or (protected carboxy)phenyl group such 4-carboxyphenyl; a mono- or di (hydroxymethyl)phenyl or (protected hydroxymethyl)phenyl such as 3-(protected hydroxymethyl)phenyl or 3,4-di (hydroxymethyl)phenyl; a mono- or di (aminomethyl)phenyl or (protected aminomethyl)phenyl such as 2-(aminomethyl)phenyl or 2,4-(protected aminomethyl)phenyl; or a mono- or di (N-(methylsulfonylamino)) phenyl such as 3-(N-methylsulfonylamino) phenyl. Also, the substituents, such as in a disubstituted phenyl groups, can be the same or different, for example, 3-methyl-4-hydroxyphenyl, 3-chloro-4-hydroxyphenyl, 2-methoxy-4-bromophenyl, 4-ethyl-2-hydroxyphenyl, 3-hydroxy-4-nitrophenyl, 2-hydroxy-4-chlorophenyl, as well as for trisubstituted phenyl groups where the substituents are different, as for example 3-methoxy-4-benzyloxy-6-methyl sulfonylamino, 3-methoxy-4-benzyloxy-6-phenyl sulfonylamino, and tetrasubstituted phenyl groups where the substituents are different such as 3-methoxy-4-benzyloxy-5-methyl-6-phenyl sulfonylamino. Particular substituted phenyl groups are 2-chlorophenyl, 2-aminophenyl, 2-bromophenyl, 3-methoxyphenyl, 3-ethoxy-phenyl, 4-benzyloxyphenyl, 4-methoxyphenyl, 3-ethoxy-4-benzyloxyphenyl, 3,4-diethoxyphenyl, 3-methoxy-4-benzyloxyphenyl, 3-methoxy-4-(1-chloromethyl)benzyloxy-phenyl, 3-methoxy-4-(1-chloromethyl)benzyloxy-6-methyl sulfonyl aminophenyl groups. Fused aryl rings may also be substituted with the substituents specified herein, for example with 1, 2 or 3 substituents, in the same manner as substituted alkyl groups.

“Heterocyclic group”, “heterocyclic”, “heterocycle”, “heterocyclyl”, “heterocycloalkyl” or “heterocyclo” alone and when used as a moiety in a complex group, are used interchangeably and refer to any cycloalkyl group, i.e., mono-, bi-, or tricyclic, saturated or unsaturated, non-aromatic hetero-atom-containing ring systems having the number of atoms designated, or if no number is specifically designated then from 5 to about 14 atoms, where the ring atoms are carbon and at least one heteroatom and usually not more than four (nitrogen, sulfur or oxygen). Included in the definition are any bicyclic groups where any of the above heterocyclic rings are fused to an aromatic ring (i.e., an aryl (e.g., benzene) or a heteroaryl ring). In a particular embodiment the group incorporates 1 to 4 heteroatoms. Typically, a 5-membered ring has 0 to 1 double bonds and 6- or 7-membered ring has 0 to 2 double bonds and the nitrogen or sulfur heteroatoms may optionally be oxidized (e.g. SO, SO₂), and any nitrogen heteroatom may optionally be quaternized. Particular non-aromatic heterocycles include morpholinyl (morpholino), pyrrolidinyl, oxiranyl, indolinyl, isoindolinyl, tetrahydroquinolinyl, tetrahydroisoquinolinyl, oxetanyl, tetrahydrofuranyl, 2,3-dihydrofuranyl, 2H-pyranyl, tetrahydropyranyl, aziridinyl, azetidinyl, 1-methyl-2-pyrrolyl, piperazinyl and piperidinyl. The term heterocyclo includes both “unsubstituted heterocyclos” and “substituted heterocyclos” (unless the context clearly indicates otherwise), the latter of which refers to heterocyclo moieties having substituents replacing one or more hydrogens on one or more (usually no more than six) atoms of the heterocyclo backbone. Such substituents are independently selected from the group consisting of: halo (e.g., I, Br, Cl, F), hydroxy, amino, cyano, alkoxy (such as C₁-C₆ alkoxy), aryloxy (such as phenoxy), nitro, carboxyl, oxo, carbamoyl, alkyl (such as trifluoromethyl), —OCF₃, aryl, alkylsulfonyl, and arylsulfonyl.

“Heteroaryl” alone and when used as a moiety in a complex group refers to any aryl group, i.e., mono-, bi-, or tricyclic aromatic ring system having the number of atoms designated, or if no number is specifically designated then at least one ring is a 5-, 6- or 7-membered ring and the total number of atoms is from 5 to about 14 and containing from one to four heteroatoms selected from the group consisting of nitrogen, oxygen, and sulfur (Lang's Handbook of Chemistry, supra). Included in the definition are any bicyclic groups where any of the above heteroaryl rings are fused to a benzene ring. The following ring systems are examples of the heteroaryl (whether substituted or unsubstituted) groups denoted by the term “heteroaryl”: thienyl (alternatively called thiophenyl), furyl, imidazolyl, pyrazolyl, thiazolyl, isothiazolyl, oxazolyl, isoxazolyl, triazolyl, thiadiazolyl, oxadiazolyl, tetrazolyl, thiatriazolyl, oxatriazolyl, pyridyl, pyrimidyl, pyrazinyl, pyridazinyl, thiazinyl, oxazinyl, triazinyl, thiadiazinyl, oxadiazinyl, dithiazinyl, dioxazinyl, oxathiazinyl, tetrazinyl, thiatriazinyl, oxatriazinyl, dithiadiazinyl, imidazolinyl, dihydropyrimidyl, tetrahydropyrimidyl, tetrazolo[1,5-b]pyridazinyl and purinyl, as well as benzo-fused derivatives, for example benzoxazolyl, benzofuryl, benzothienyl, benzothiazolyl, benzothiadiazolyl, benzotriazolyl, benzoimidazolyl and indolyl. The term heteroaryl includes both “unsubstituted heteroaryls” and “substituted heteroaryls” (unless the context clearly indicates otherwise), the latter of which refers to heteroaryl moieties having substituents replacing one or more hydrogens on one or more (usually no more than six) atoms of the heteroaryl backbone. Such substituents are independently selected from the group consisting of: halo (e.g., I, Br, Cl, F), hydroxy, amino, cyano, alkoxy (such as C₁-C₆ alkoxy), aryloxy (such as phenoxy), nitro, carboxyl, oxo, carbamoyl, alkyl (such as trifluoromethyl), —OCF₃, aryl, alkylsulfonyl, and arylsulfonyl. Particular “heteroaryls” include; 1H-pyrrolo[2,3-b]pyridine, 1,3-thiazol-2-yl, 4-(carboxymethyl)-5-methyl-1,3-thiazol-2-yl, 1,2,4-thiadiazol-5-yl, 3-methyl-1, 2,4-thiadiazol-5-yl, 1,3,4-triazol-5-yl, 2-methyl-1,3,4-triazol-5-yl, 2-hydroxy-1,3,4-triazol-5-yl, 2-carboxy-4-methyl-1,3,4-triazol-5-yl, 1,3-oxazol-2-yl, 1,3,4-oxadiazol-5-yl, 2-methyl-1,3,4-oxadiazol-5-yl, 2-(hydroxymethyl)-1,3,4-oxadiazol-5-yl, 1,2,4-oxadiazol-5-yl, 1,3,4-thiadiazol-5-yl, 2-thiol-1,3,4-thiadiazol-5-yl, 2-(methylthio)-1, 3,4-thiadiazol-5-yl, 2-amino-1,3,4-thiadiazol-5-yl, 1H-tetrazol-5-yl, 1-methyl-1H-tetrazol-5-yl, 1-(1-(dimethylamino) eth-2-yl)-1H-tetrazol-5-yl, 1-(carboxymethyl)-1H-tetrazol-5-yl, 1-(methylsulfonic acid)-1H-tetrazol-5-yl, 2-methyl-1H-tetrazol-5-yl, 1,2,3-triazol-5-yl, 1-methyl-1,2,3-triazol-5-yl, 2-methyl-1,2,3-triazol-5-yl, 4-methyl-1,2,3-triazol-5-yl, pyrid-2-yl N-oxide, 6-methoxy-2-(n-oxide)-pyridaz-3-yl, 6-hydroxypyridaz-3-yl, 1-methylpyrid-2-yl, 1-methylpyrid-4-yl, 2-hydroxypyrimid-4-yl, 1,4,5,6-tetrahydro-5,6-dioxo-4-methyl-as-triazin-3-yl, 1,4,5,6-tetrahydro-4-(formylmethyl)-5,6-dioxo-as-triazin-3-yl, 2,5-dihydro-5-oxo-6-hydroxy-astriazin-3-yl, 2,5-dihydro-5-oxo-6-hydroxy-as-triazin-3-yl, 2,5-dihydro-5-oxo-6-hydroxy-2-methyl-astriazin-3-yl, 2,5-dihydro-5-oxo-6-hydroxy-2-methyl-as-triazin-3-yl, 2,5-dihydro-5-oxo-6-methoxy-2-methyl-as-triazin-3-yl, 2,5-dihydro-5-oxo-as-triazin-3-yl, 2,5-dihydro-5-oxo-2-methyl-as-triazin-3-yl, 2,5-dihydro-5-oxo-2,6-dimethyl-as-triazin-3-yl, tetrazolo[1,5-b]pyridazin-6-yl and 8-aminotetrazolo[1,5-b]-pyridazin-6-yl. An alternative group of “heteroaryl” includes: 4-(carboxymethyl)-5-methyl-1,3-thiazol-2-yl, 1,3,4-triazol-5-yl, 2-methyl-1,3,4-triazol-5-yl, 1H-tetrazol-5-yl, 1-methyl-1H-tetrazol-5-yl, 1-(1-(dimethylamino) eth-2-yl)-1H-tetrazol-5-yl, 1-(carboxymethyl)-1H-tetrazol-5-yl, 1-(methylsulfonic acid)-1H-tetrazol-5-yl, 1,2,3-triazol-5-yl, 1,4,5,6-tetrahydro-5,6-dioxo-4-methyl-as-triazin-3-yl, 1,4,5,6-tetrahydro-4-(2-formylmethyl)-5,6-dioxo-as-triazin-3-yl, 2,5-dihydro-5-oxo-6-hydroxy-2-methyl-as-triazin-3-yl, 2,5-dihydro-5-oxo-6-hydroxy-2-methyl-as-triazin-3-yl, tetrazolo[1,5-b]pyridazin-6-yl, and 8-aminotetrazolo[1,5-b]pyridazin-6-yl.

“IAP Inhibitor” or “IAP antagonist” means a compound which interferes with the physiological function of an IAP protein, including the binding of IAP proteins to caspase proteins, for example by reducing or preventing the binding of IAP proteins to caspase proteins, or which reduces or prevents the inhibition of apoptosis by an IAP protein, or which binds to an IAP BIR domain in a manner similar to the amino terminal portion of Smac.

As used herein, the terms “pharmaceutically acceptable”, “physiologically tolerable” and grammatical variations thereof, as they refer to compositions, excipients, carriers, diluents and reagents, are used interchangeably and represent that the materials can be administered to a human being.

“Pharmaceutically acceptable salts” include both acid and base addition salts.

“Pharmaceutically acceptable acid addition salt” refers to those non-toxic salts which retain the biological effectiveness and essential properties of the free bases and which are not biologically or otherwise undesirable, and are formed with inorganic acids and with organic acids. The acid addition salts of the basic compounds are prepared by contacting the free base form of the compound with a sufficient amount of the desired acid to produce the salt in the conventional manner. The free base form may be regenerated by contacting the salt form with a base and isolating the free base in the conventional manner. The free base forms generally differ from their respective salt forms somewhat in certain physical properties such as solubility in polar solvents.

“Pharmaceutically acceptable base addition salts” are formed with metals or amines, such as alkali and alkaline earth metal hydroxides, or with organic amines. The base addition salts of acidic compounds are prepared by contacting the free acid form with a sufficient amount of the desired base to produce the salt in the conventional manner. The free acid form may be regenerated by contacting the salt form with an acid and isolating the free acid in a conventional manner. The free acid forms usually differ from their respective salt forms somewhat in certain physical properties such as solubility in polar solvents.

As used herein “subject” or “patient” refers to an animal or mammal including, but not limited to, human, dog, cat, horse, cow, pig, sheep, goat, chicken, monkey, rabbit, rat, and mouse.

As used herein, the term “therapeutic” refers to the amelioration of the prevention of, an improvement of, or a delay in the onset of one or more symptoms of an unwanted condition or disease of a patient. Embodiments of the present invention are directed to therapeutic treatments by promoting apoptosis, and thus cell death.

The terms “therapeutically effective amount” or “effective amount”, as used herein, means an amount of a compound, or a pharmaceutically acceptable salt thereof, sufficient to inhibit, halt, delay the onset of, or cause an improvement in the disease being treated when administered alone or in conjunction with another pharmaceutical agent for treatment in a particular subject or subject population. For example in a human or other mammal, a therapeutically effective amount can be determined experimentally in a laboratory or clinical setting, or may be the amount required by the guidelines of the United States Food and Drug Administration, or equivalent foreign agency, for the particular disease and subject being treated.

### DETAILED DESCRIPTION OF THE INVENTION

It has been demonstrated in accordance with the present invention that the IAP-binding compounds of the present invention are capable of potentiating apoptosis of cells.

Compounds of the present invention can be used in their free base or free acid forms or in the form of their pharmaceutically-acceptable salts. In the practice of the present invention, compounds of the present invention in their free base or free acid forms generally will have a molecular weight of 1000 or below, most often a molecular weight of 800 or below and often a molecular weight of 600 or below.

The following preparations and schemes are illustrative of synthesis of compounds of the present invention. Abbreviations which are used throughout these schemes and in the application generally, are identified in the following table:

| Abbreviation | Meaning |
|---|---|
| ACN | Acetonitrile |
| Cbz and Z | Benzyloxycarbonyl |
| Boc | tert-butyloxycarbonyl |
| and/or | |
| boc | |
| THF | Tetrahydrofuran |
| DCM | Dichloromethane |
| DDQ | 2,3-dichloro-5,6-dicyano-1,4- |
| | benzoquinone |
| mCPBA | 3-chloroperbenzoic acid |
| Hex | Hexanes |
| HPLC | high performance liquid |
| | chromatography |
| TLC | thin layer chromatography |
| EtOAc | ethyl acetate |
| Ph | Phenyl |
| HATU | 2-(7-Aza-1H-benzotriazole-1-yl)- |
| | 1,1,3,3-tetramethyluronium |
| | hexafluorophosphate |
| Me | Methyl* |
| iPr | Iso-propyl |
| cPr | Cyclopropyl |
| (2R-EtOMe) and/or R-MeCHOMe | 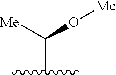 |
| TBAF | tetrabutyl ammonium fluoride |
| OMs | Methanesulfonyloxy |
| TBDMSCl | tert-butyl-dimethyl-silyl chloride |
| Ph₃P | triphenylphosphine |
| n-Bu | Normal butyl |
| Swern[O] | Swern Oxidation |
| TBA-Cl | Tetra-n-butyl ammonium chloride |
| NP-HPLC | Normal phase-high performance |
| | liquid chromatography |
| | N-3-(dimethylaminopropyl)-N′- |
| | ethylcarbodiimide hydrochloride |
| EDCI | 1-Ethyl-3-(3- |
| | Dimethylaminopropyl)carbodiimide- |
| | HCl |
| TES | triethylsilane |
| NMP | N-methylpyrrolidinone |
| DIAD | diisopropyl azo |
| | dicarboxylate |
| DIBAL | Diisobutylaluminium |
| | hydride |
| DMAP | 4-dimethylamino |
| | pyridine |
| DMF | Dimethylformamide |
| DMSO | dimethyl sulfoxide |
| TFA | trifluoroacetic acid |
| HOAc or | acetic acid |
| AcOH | |
| DIPEA | Diisopropylethylamine |
| NMM | N-methylmorpholine |
| NCS | N-chlorosuccinimide |
| TEA (Et₃N) | Triethylamine |
| MsCl | Methane- |
| | sulfonylchloride |
| Et | Ethyl |
| tBu or tert-Bu | tert-butyl |
| cHex | Cyclohexyl |
| (2R-EtOH) and/or R-MeCHOH | 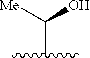 |
| MsCl | Methanesulfonyl |
| | chloride |
| OTs | —O—SO₂—Ph—Me |
| OTBS | tert-butyl-dimethyl- |
| | silanyloxy |
| Ac | 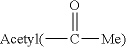 |
| DMA | Dimethylamine |
| HWE | Honer-Wadsworth- |
| | Emmons reaction |
| DMS | Dimethylsulfide |
| Meldrum's Acid | 2,2-dimethyl-1,3- |
| | dioxane-4,6-dione |
| Imid. | Imidazole |

*Alternatively, as is commonly accepted convention, a vacant terminal bond may also be used to indicate a methyl.

Abbreviations for NMR data reported in the following examples are as follows: s=singlet, d=doublet, t=triplet, q=quartet, m=multiplet, dd=doublet of doublets, ddd=doublet of doublet of doublets, dt=doublet of triplets, app=apparent, br=broad, J indicates the NMR coupling constant measured in Hertz.

The binding affinities of the compounds listed below to XIAP BIR-3 or cIAP-1 BIR-3 were determined substantially as described by Nikolovska-Coleska, Z. et. al. (Analytical Biochemistry (2004), vol. 332:261-273) using as the fluorogenic substrate the fluorescently labeled peptide AbuRPF-K(5-Fam)-NH2. The binding affinities of the compounds are reported as a Kd value. Briefly, various concentrations of test peptides were mixed with 5 nM of the fluorescently labeled peptide (i.e., a mutated N-terminal Smac peptide—AbuRPF-K(5-Fam)-NH2) and 40 nM of the BIR3 for 15 min at RT in 100 mL of 0.1M Potassium Phosphate buffer, pH 7.5 containing 100 mg/ml bovine g-globulin. Following incubation, the polarization values (mP) were measured on a Victor2V (available from PerkinElmer Life Sciences) using a 485 nm excitation filter and a 520 nm emission filter. The reported Kd values are supplied as ranges (A=<0.1 μM, B=0.1 μM to C=>1 μM to 10 μM, D=>10 μM) and, unless otherwise indicated, are the Kd for XIAP BIR-3.

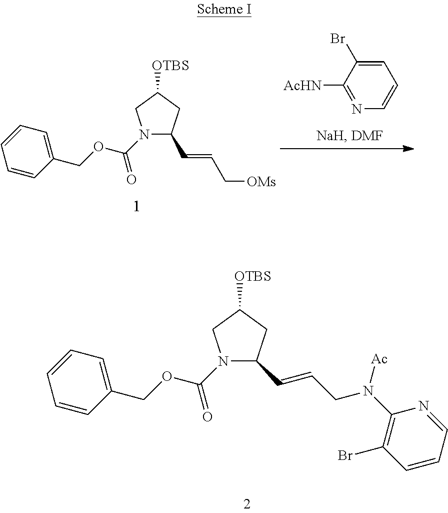

2-{3-[Acetyl-(3-bromo-pyridin-2-yl)-amino]-propenyl}-4-(tert-butyl-dimethyl-silanyloxy)-pyrrolidine-1-carboxylic acid benzyl ester (2): Under a nitrogen atmosphere at 0° C., NaH (0.89 g, 23.0 mmol) was added in portions to a solution containing 2-acetylamino-3-bromopyridine (4.12 g, 19.2 mmol) in DMF (30 mL). After 15 min at 0° C. for and 1 h at ambient temperature the reaction mixture was recooled to 0° C. and a solution containing 1 (8.99 g, 19.2 mmol. See: Ohtake, N., et al. J. Antibiotics 1997, 50, 586-597) in DMF (10 mL) was added dropwise. The reaction mixture was then stirred at ambient temperature for 2 h at which point TLC analysis revealed complete consumption of 1 [1:1 hexanes/EtOAc, R_{f}(1)=0.6; R_{f}(2)=0.3]. The reaction mixture was cooled to 0° C. followed by the dropwise addition of saturated aqueous NH₄Cl. The product was extracted with diethyl ether. The combined ether extracts were washed with water, brine, dried over anhydrous Na₂SO₄, filtered and concentrated. The crude product was purified by flash silica gel chromatography (20% EtOAc/hexanes) to afford 6.0 g (54%) of 2 as an white solid. ¹H NMR (CDCl₃, 300 MHz) δ 7.4-7.2 (m, 5H), 5.6-5.4 (m, 2H), 5.0 (s, 2H), 4.4-4.2 (m, 4H), 3.5-3.2 (m, 2H), 1.8 (s, 3H), 1.6 (s, 2H), 0.9 (s, 6H), 0.1 (s, 9H) ppm.

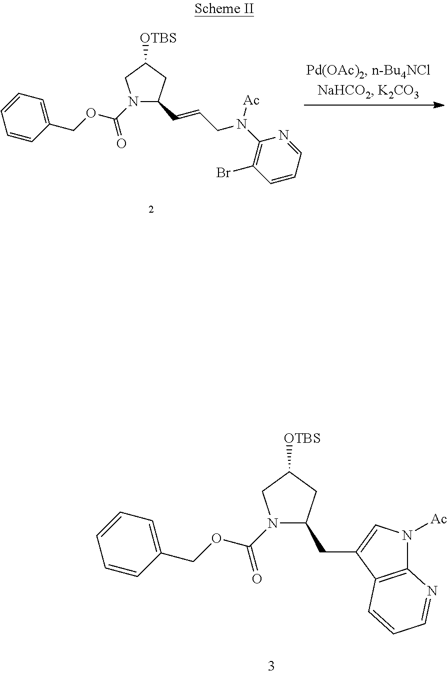

4-Acetoxy-2-(1-acetyl-1H-pyrrolo[2,3-b]pyridine-3-ylmethyl)-pyrrolidine-1-carboxylic acid benzyl ester (3): Under a nitrogen atmosphere, a solution containing 2 (5.92 g, 10.1 mmol) in anhydrous DMF (50 mL) was charged with (n-Bu)₄NCl (2.8 g, 10.1 mmol), K₂CO₃ (1.4 g, 10.1 mmol), NaHCO₂ (0.68 g, 10.1 mmol), and Pd(OAc)₂ (0.045 g, 0.20 mmol) at ambient temperature. The heterogeneous mixture was immersed in a pre-heated (85° C.) oil bath. After 3 h, TLC analysis revealed some 2 remained therefore additional Pd(OAc)₂ catalyst (0.01 g) was added. After an additional 1 h of heating, 2 was completely consumed by TLC analysis [1:1 EtOAc/hexanes, R_{f}(2)=0.3; R_{f}(3)=0.8]. The warm reaction mixture was cooled in an ice bath then diluted with diethyl ether and filtered through a pad of Celite®. The solids were washed with diethyl ether and the filtrate was washed several times with water to remove excess DMF, then washed once with brine, dried over anhydrous Na₂SO₄, filtered, and concentrated to afford 5.1 g of crude 3 which was purified by flash silica gel chromatography (20% EtOAc/hexanes) to afford 3.0 g (59%) of 3 as an white solid. ¹H NMR (CDCl₃, 300 MHz) δ 5.18 (m, 1H), 7.60 (m, 1H), 7.18 (m, 1H), 7.05 (dt, J=2.4, 8.7 Hz, 1H), 4.13 (m, 1H), 3.41 (m, 1H), 3.33 (m, 2H), 3.17 (app dd, J=14.1, 38.1 Hz, 1H), 2.61 (s, 3H), 1.83 (m, 3H), 1.69 (m, 1H), 1.49 (s, 9H) ppm.

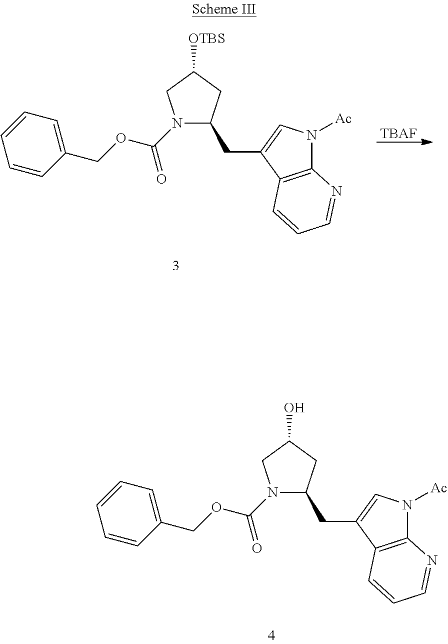

2-(1-Acetyl-1H-pyrrolo[2,3-b]□-pyridine-3-ylmethyl)-4-hydroxy-pyrrolidine-1-carboxylic acid benzyl ester (4): To a solution containing 3 (2.99 g, 5.88 mmol) in THF (20 mL) at 0° C. was added a solution of TBAF (1 M in THF, 11.8 mL, 11.8 mmol) in a dropwise fashion. After 1.5 h, TLC analysis revealed complete consumption of 3 [1:1 hexanes/EtOAc, R_{f}(3)=0.64; R_{f}(4)=0.3]. The solvent was removed in vacuo and the residue was dissolved in EtOAc and washed with water, brine, dried over anhydrous Na₂SO₄, filtered, and concentrated to afford 2.11 g of crude 4 which was used without further purification.

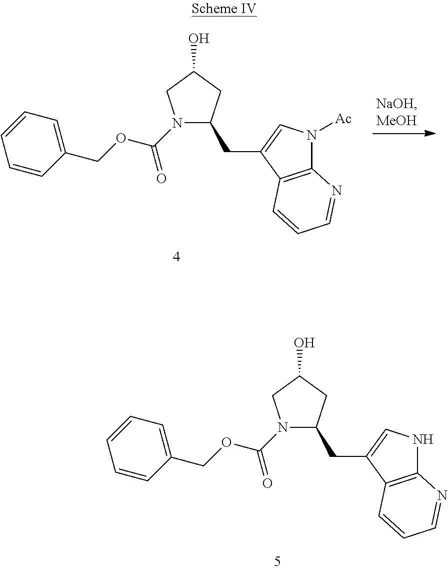

4-Hydroxy-2-(1H-pyrrolo[2,3-b]pyridin-3-ylmethyl)-pyrrolidine-1-carboxylic acid benzyl ester (5): To a solution containing 4 (2.11 g, 5.36 mmol) in MeOH (30 mL) at 0° C. was added 1M NaOH (8.1 mL, 8.05 mmol) in a dropwise fashion. After 1 h, TLC analysis revealed complete consumption of 4 [EtOAc, R_{f}(4)=0.4; R_{f}(5)=0.2]. The MeOH was removed in vacuo and the residue was dissolved in EtOAc, washed with dilute aqueous HCl, water, brine, dried over anhydrous Na₂SO₄, filtered and concentrated to afford 1.99 g of crude 5 which was used in the next step without further purification.

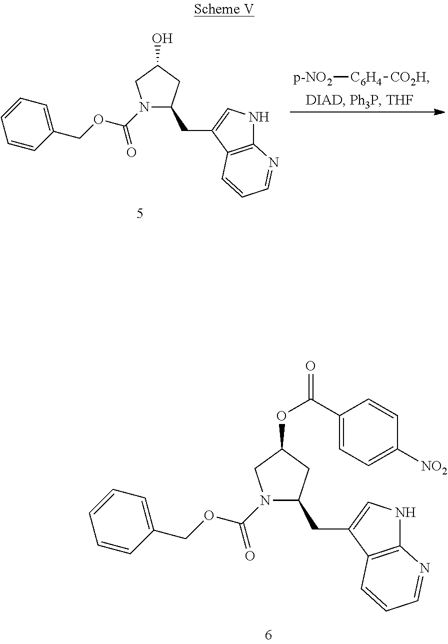

4-(4-Nitro-benzoyloxy)-2-(1H-pyrrolo[2,3-b]pyridine-3-ylmethyl)-pyrrolidine-1-carboxylic acid benzyl ester (6): To a solution containing 5 (1.99 g, 5.66 mmol), p-nitrobenzoic acid (1.23 g, 7.36 mmol), and Ph₃P (2.07 g, 7.92 mmol) in THF (35 mL) at 0° C. was added DIAD (1.6 mL, 8.2 mmol). After the addition was complete, the ice bath was removed and the reaction mixture was stirred at ambient temperature for 2 h at which point TLC analysis revealed complete consumption of 5 [EtOAc, R_{f}(5)=0.2; R_{f}(6)=0.6 ]. The solvent was removed in vacuo and the residue was dissolved in EtOAc, washed with saturated aqueous NaHCO₃, brine, dried over anhydrous Na₂SO₄, filtered, and concentrated to afford 7 g of crude 6 which was purified by flash silica gel chromatography (20% EtOAc/hexanes) to obtained 2.68 g of 6 (95%) as a white solid ¹H NMR (CDCl₃, 300 MHz): δ 8.3 (d, J=35 Hz, 2H), 7.6 (d, J=35 Hz, 2H), 7.2 (m, 5H), 7.0 (s, 1H), 5.2 (s, 2H), 4.4-3.2 (m, 3H), 3.0-2.9 (m, 1H), 2.2 (s, 2H), 1.9 (s, 2H) ppm.

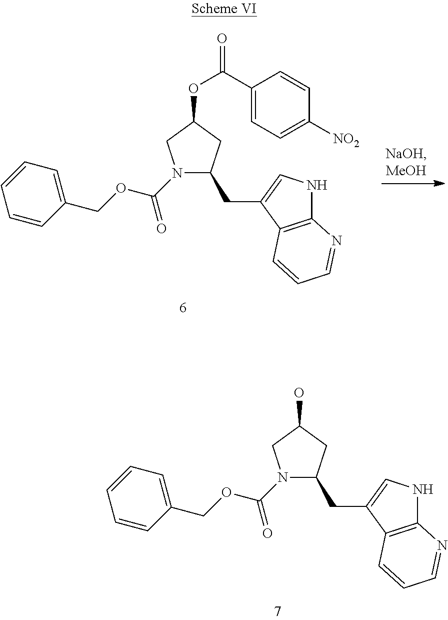

4-Hydroxy-2-(1H-pyrrolo[2,3-b]□pyridine-3-ylmethyl)-pyrrolidine-1-carboxylic acid benzyl ester (7): To a solution containing 6 (2.8 g, 5.6 mmol) in a 3:1 mixture of MeOH/DCM (40 mL) at 0′C was added 1N NaOH (8.5 mL) and the reaction mixture was stirred at ambient temperature for 15 min when TLC analysis revealed complete consumption of 6 [1:1 EtOAc/hexanes; R_{f}(6)=0.3; R_{f}(7)=0.02]. The solvent was removed in vacuo and the residue was dissolved in EtOAc, washed with dilute aqueous HCl, water, brine, dried over anhydrous Na₂SO₄, filtered, and concentrated to afford 2.7 g of crude 7 which was purified by flash silica gel chromatography (50% EtOAc/hexanes) to obtained 1.6 g of 7 (94%) as a white solid. ¹H NMR (CDCl₃, 300 MHz): δ 8.5 (m, 2H), 7.4 (s, 5H), 7.0 (m, 2H), 5.2 (s, 2H), 4.3 (s, 1H), 4.2 (m, 1H), 3.65-3.8 (m, 1H), 3.5-3.3 (m, 2H), 3.2-3.0 (m, 1H), 1.9-2.0 (m, 3H) ppm.

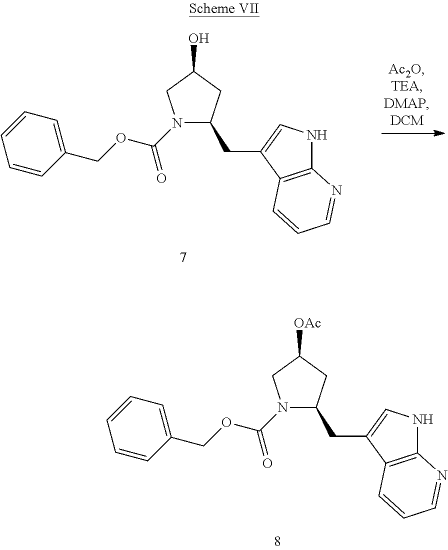

4-Acetoxy-2-(1H-pyrrolo[2,3-b]□pyridine-3-ylmethyl)-pyrrolidine-1-carboxylic acid benzyl ester (8): To a solution containing 7 (1.6 g, 4.55 mmol) in DCM (20 mL) at 0° C. was added triethylamine (1.3 mL, 9.1 mmol) followed by the dropwise addition of Ac₂O (0.64 mL, 6.82 mmol) and a catalytic amount of DMAP. The reaction mixture was stirred under a nitrogen atmosphere for 30 min at which point TLC analysis revealed the complete consumption of 7 [EtOAc: R_{f}(7)=0.2, R_{f}(8)=0.4]. The reaction mixture was transferred to a separatory funnel, diluted with DCM, washed successively with water, dilute aqueous HCl, water, and brine, then dried over anhydrous Na₂SO₄, filtered, and concentrated to afford 1.96 g of crude 8 which was used without further purification.

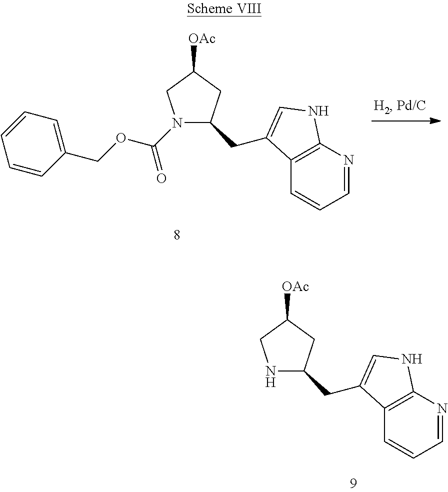

Acetic acid 5-(1H-pyrrolo[2,3-b]□pyridine-3-ylmethyl)-pyrrolidin-3-yl ester (9): To a solution containing 8 (0.5 g, 1.27 mmol) in a 1:1 mixture of MeOH/EtOAc (14 mL) was added catalytic amount of 5% Pd-on-C and the heterogeneous mixture was placed on a Parr apparatus at 50 PSI (3.4 atm) hydrogen pressure for 2 h. TLC analysis revealed the complete consumption of 8 [EtOAc: R_{f}(8)=0.4, R_{f}(9)=0.04]. The Pd-on-C catalyst was removed by filtration through a pad of Celite® and the clarified filtrate was concentrated in vacuo. LC/MS confirmed the formation of 9: mass spectrum, m/z=260.1 [(M+H)+]. The crude product (9) was used without further purification.

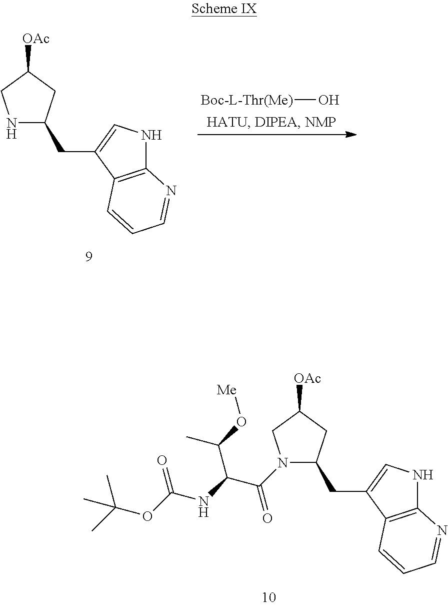

Acetic acid 1-(2-tert-butoxycarbonylamino-3-methoxy-butyryl)-5-(1H-pyrrolo[2,3-]pyridine-3-ylmethyl)-pyrrolidin-3-yl ester (10): To a solution containing crude 9 (0.33 g, 1.27 mmol) and Boc-L-Thr(Me)-OH (0.30 g, 1.27 mmol) in NMP (10 mL) at 0° C. was added DIPEA (0.22 mL, 1.27 mmol) followed by HATU (0.48 g, 1.27 mmol) and the reaction mixture was stirred to ambient temperature over 12 h at which point TLC analysis revealed the complete consumption of 9 [1:1 EtOAc/hexanes; R_{f}(9)=0.01, R_{f}(10)=0.4]. The reaction mixture was diluted with diethyl ether and washed successively with dilute aqueous HCl, water, saturated aqueous NaHCO₃, water (5×), brine, and dried over anhydrous Na₂SO₄, filtered, and concentrated to afford 0.5 g of crude 10 which was purified by flash silica gel chromatography (20% EtOAc/hexanes) to provide 0.37 g (61%) of 10 as a white solid. ¹H NMR (CDCl₃, 300 MHz): δ 9.2 (s, 1H), 8.4-8.2 (m, 2H), 7.1 (s, 1H), 5.6 (d, J=10.7 Hz, 1H), 5.3 (s, 1H), 4.6-4.4 (m, 2H), 4.0 (m, 2h), 3.9 (m, 1H), 3.6 (m, 1H), 3.4 (s, 3H), 2.8 (dd, J=16 Hz, 10 Hz). 2.1 (s, 3H), 1.4 (s, 9H), 1.1 (d, J=10.7 Hz, 3H) ppm.

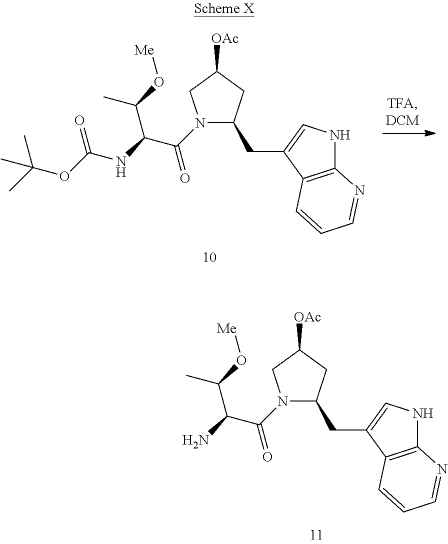

Acetic acid 1-(2-amino-3-methoxy-butyryl)-5-(1H-pyrrolo[2,3-b]pyridine-3-ylmethyl)-pyrrolidin-3-yl ester (11): To a solution of 10 (0.20 g, 0.42 mmol) in DCM (16 mL) at 0° C. was added TFA (4 mL). After 45 min, TLC analysis revealed the complete consumption of 10. [1:1 EtOAc/hexanes; R_{f}(10)=0.5, R_{f}(11)=0.04]. After concentration in vacuo, the residue was dissolved in EtOAc and washed successively with saturated aqueous NaHCO₃, water, and brine, then dried over anhydrous Na₂SO₄, filtered, and concentrated to afford 0.16 g crude 11 which was used without further purification.

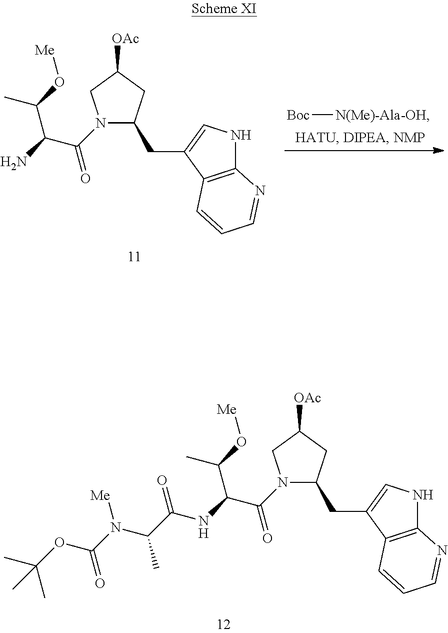

Acetic acid 1-{2-[2-(tert-butoxycarbonyl-methyl-amino)-propionylamino]-3-methoxy-butyryl}-5-(1H-pyrrolo[2,3-b]pyridine-3-ylmethyl)-pyrrolidin-3-yl ester (13): To a solution containing 11 (0.16 g, 0.42 mmol) and Boc-L-N(Me)-Ala-OH (0.09 g, 0.42 mmol) in NMP (5 mL) at 0° C. was added DIPEA (0.07 mL, 0.42 mmol) followed by HATU (0.16 g, 0.42 mmol). The reaction mixture was allowed to slowly warm to ambient temperature. After 12 h, TLC analysis revealed the complete consumption of 11 [EtOAc; R_{f}(11)=0.1, R_{f}(12)=0.4]. The reaction mixture was diluted with diethyl ether then washed successively with dilute aqueous HCl, water, saturated aqueous NaHCO₃, water (5×), and brine. The organic extract was dried over anhydrous Na₂SO₄, filtered, and concentrated to afford 0.23 g of 12 which was used without further purification.

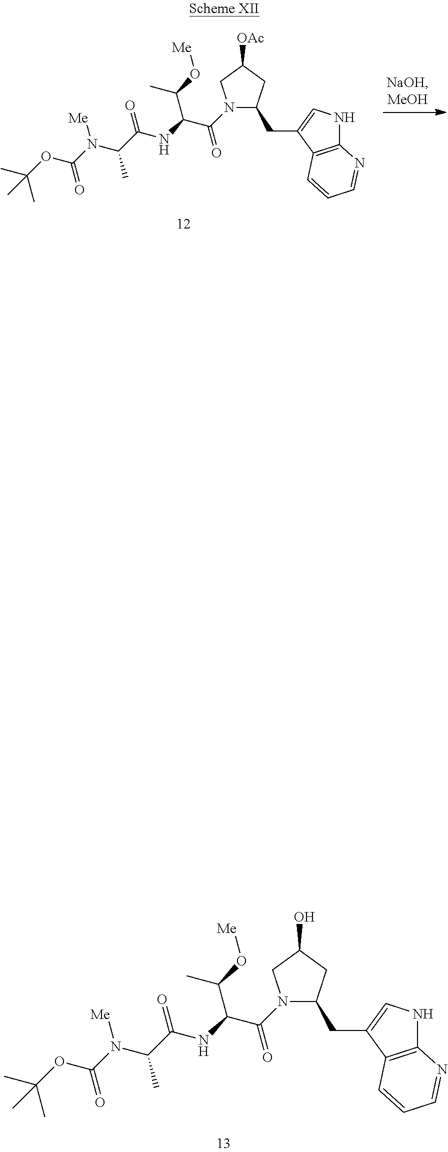

(1-{1-[4-Hydroxy-2-(1H-pyrrolo[2,3-b]pyridine-3-ylmethyl)-pyrrolidine-1-carbonyl]-2-methyl-propylcarbamoyl}-ethyl)-methyl-carbamic acid tert-butyl ester (13): To a solution containing 12 (0.16 g, 0.28 mmol) in a 5:1 mixture of MeOH/DCM (6 mL) was added 1M NaOH (0.3 mL, 0.3 mmol) at 0° C. After 90 min, TLC analysis revealed the complete consumption of 12 [20% MeOH/DCM; R_{f}(12)=0.55, R_{f}(13)=0.51]. Following removal of the solvent in vacuo, the residue was dissolved in EtOAc and washed successively with dilute aqueous HCl, water, and brine. The organic extract was dried over anhydrous Na₂SO₄, filtered, and concentrated to afford 0.15 g of 13 which was used without further purification.

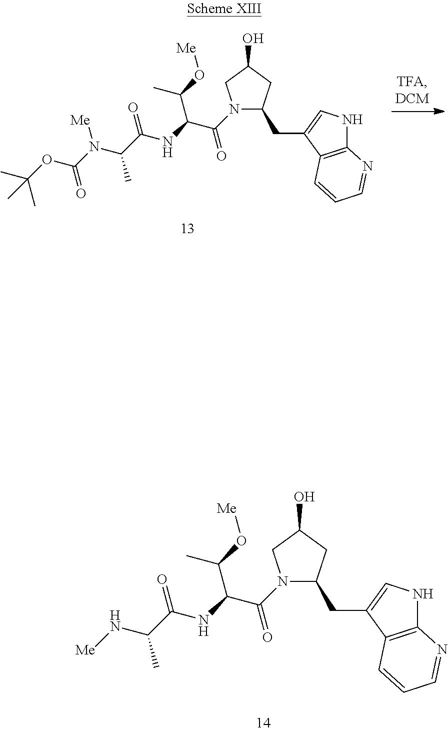

N-{1-[4-Hydroxy-2-(1H-pyrrolo[2,3-b]pyridin-3-ylmethyl)-pyrrolidine-1-carbonyl]-2-methoxy-propyl}-2-methylamino-propionamide (14): To a solution containing 13 (0.29 g, 0.56 mmol) in DCM (16 mL) at 0° C. was added TFA (4 mL). After 1.5 h, TLC analysis revealed the complete consumption of 13 [20% MeOH/DCM, R_{f}(13)=0.5, R_{f}(14)=0.2]. The reaction mixture was concentrated in vacuo and the residue was dissolved in EtOAc and washed successively with saturated aqueous NaHCO₃, water, and brine. The organic extract was dried over anhydrous Na₂SO₄, filtered, and concentrated. The crude product was purified by C18 RP-HPLC [Solvent A: Water w/0.1% v/v HOAc, Solvent B: ACN w/0.1% v/v HOAc. Dynamax Microsorb C18 60 Å, 8μ, 41.4 mm×25 cm (Varian, Inc); Flow: 40 mL/min; Detector: 254 nm). The product-containing fractions were pooled, frozen, and lyophilized to afford 0.13 g of 14 (identified as Compound A in Table 1). ¹H NMR (CDCl₃, 300 MHz): δ 8.26 (m, 2H), 7.93 (m, 1H), 7.2 (m, 2H), 4.7 (m, 1H), 4.55 (m, 2H), 4.0 (m, 1H), 3.7 (m, 2H), 3.7 (m, 1H), 3.4 (s, 3H), 3.35 (m, 1H), 3.19 (app t, 1H), 3.0 (app t, 1H), 2.42 (s, 3H), 2.4 (m, 1H), 2.19 (s, 1H), 1.35 (d, J=11, 3H), 1.3 (d, J=11, 3H) ppm.

Using the general procedures outlined in Schemes I through XIII and the appropriate amino acid analogues to the amino acid reagents Boc-Thr(Me)-OH and Boc-N(Me)Ala-OH, the compounds reported in Table 1 were prepared and tested for their binding affinities (Kd) to XIAP BIR-3 or cIAP-1 BIR-3.

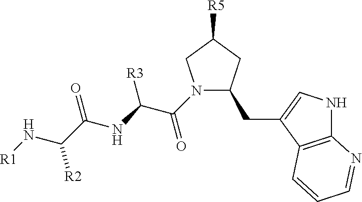

TABLE 1

| | | | | | | Observed |
|---|---|---|---|---|---|---|
| | | | | | Kd | Mass |
| Compound | R1 | R2 | R3 | R5 | (μM) | (m/z) |
| A | Me | Me | (2R-EtOMe) | (S)—OH | A | 418.5 |
| B | Me | Et | Cyclohexyl | (S)—OH | A | 456.3 |
| C | Me | Me | tent-Butyl | (S)—OH | A | 416.4 |
| D | Me | Me | (2R-EtOMe) | H | A | 401.6 |
| E | Me | Me | tert-Butyl | H | A | 399.7 |
| F | Me | Et | (2R-EtOMe) | H | A | 415.5 |
| G | Et | Et | (2R-EtOMe) | H | A | 429.5 |
| H | Et | Me | (2R-EtOMe) | H | A | 415.5 |
| I | Et | H | (2R-EtOMe) | H | B | 401.5 |
| J | Me | CH₂OH | (2R-EtOMe) | H | A | 417.5 |
| K | Et | Me | tert-Butyl | H | A | 413.6 |
| L | Me | Et | tert-Butyl | H | A | 413.6 |
| M | Et | Et | tert-Butyl | H | B | 427.7 |
| N | Et | H | tert-Butyl | H | C | 399.2 |
| O | Me | CH₂OH | tert-Butyl | H | A | 415.4 |

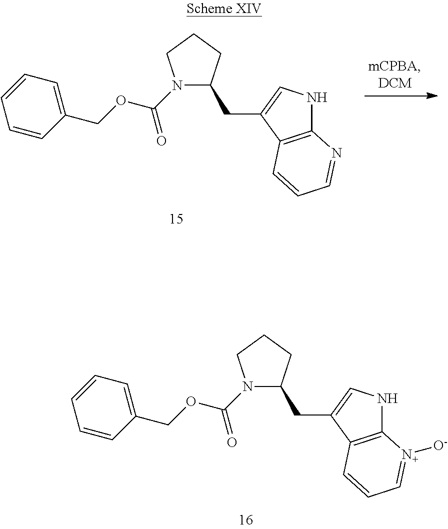

3-(1-Benzyloxycarbonyl-pyrrolidin-2-ylmethyl)-1H-pyrrolo[2,3-b]pyridine N-oxide (16): A solution containing 15 (600 mg, 1.8 mmol) in DCM (15 mL) was cooled to 0° C. mCPBA (500 mg, 1.7 mmol) was added in portions. After 2 h, the reaction mixture was diluted with DCM and washed successively with aqueous NaHCO₃ (2×) and brine, dried over anhydrous Na₂SO₄, filtered, and concentrated. The crude product was purified by flash silica gel chromatography (5% MeOH/DCM) to afford 530 mg (83%) of 16. Mass spectrum, m/z=[352.0] (M)+.

Using the general procedures outlined in Schemes I through XIV and the appropriate amino acid analogues to the amino acid reagents Cbz-Hyp-OH, Boc-Thr(Me)-OH, and Boc-N(Me)Ala-OH the compounds reported in Table 2 were prepared and tested for their binding affinities (Kd) to XIAP BIR-3 or cIAP-1 BIR-3.

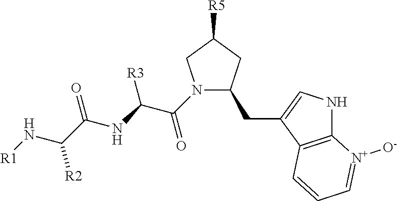

TABLE 2

| | | | | | | Observed |
|---|---|---|---|---|---|---|
| | | | | | | Mass |
| Compound | R1 | R2 | R3 | R5 | Kd (μM) | (m/z) |
| P | Me | Me | (2R-EtOMe) | H | A | 418.2 |
| Q | Et | Me | (2R-EtOMe) | H | B | 432.2 |
| R | Et | Et | (2R-EtOMe) | H | B | 446.6 |
| S | Me | Me | tert-Butyl | H | A | 416.4 |

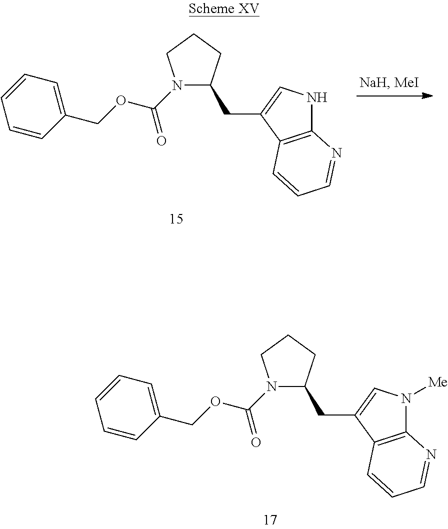

3-(1-Benzyloxycarbonyl-pyrrolidin-2-ylmethyl)-1-methyl-1H-pyrrolo[2,3-b]pyridine (17): A solution containing 15 (1.7 g, 5.07 mmol) in anhydrous THF (25 mL) was cooled to 0° C. NaH (60%, 230 mg, 6.08 mmol) was added in portions. Following the addition, the reaction mixture was warmed to ambient temperature. MeI (720 mg, 5.07 mmol) in THF (2 mL) was added dropwise. After 30 min, the reaction mixture was concentrated in vacuo and the residue was dissolved in EtOAc. The organic solution was washed successively with water and brine, dried over anhydrous Na₂SO₄, filtered, and concentrated. The crude product was purified by flash silca gel chromatography (2:1 hexane/EtOAc) to afford 1.38 g (77%) of 17. Mass spectrum, m/z=[350.0] (M+H)+.

Using the general procedures outlined in Schemes I through XIII and Scheme XV and the appropriate amino acid analogues to the amino acid reagents Cbz-Hyp-OH, Boc-Thr(Me)-OH, and Boc-N(Me)Ala-OH the compounds reported in Table 3 were prepared and tested for their binding affinities (Kd) to XIAP BIR-3 or cIAP-1 BIR-3.

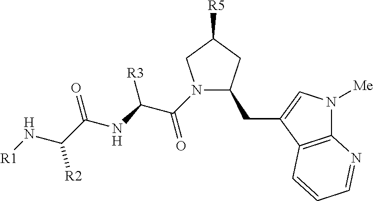

TABLE 3

| | | | | | | Observed |
|---|---|---|---|---|---|---|
| | | | | | | Mass |
| Compound | R1 | R2 | R3 | R5 | Kd (μM) | (m/z) |
| T | Me | Et | (2R-EtOMe) | H | A | 430.2 |
| U | Me | Me | (2R-EtOMe) | H | A | 416.5 |
| V | Me | Et | tert-Butyl | H | B (cIAP-1) | 428.2 |
| W | Et | Me | tert-Butyl | H | A (cIAP-1) | 428.3 |
| X | Me | Me | tert-Butyl | H | A (cIAP-1) | 414.2 |

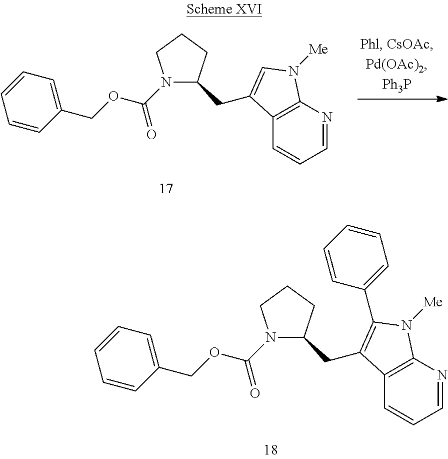

2-(1-Methyl-2-phenyl-1H-pyrrolo[2,3-b]pyridin-3-ylmethyl)-pyrrolidine-1-carboxylic acid benzyl ester (18): A mixture containing 17 (300 mg, 0.86 mmol), CsOAc (dried at 120° C. under high vacuum for 16 h, 329 mg, 1.72 mmol), Pd(OAc)₂ (1 mg, 0.5 mol %), Ph₂P (4.5 mg, 2 mol %), and PhI (211 mg, 1.03 mmol) in DMA (0.2 mL) was warmed to 125° C. After 16 h, the reaction mixture was cooled to ambient temperature and diluted with DCM. The heterogeneous mixture was filtered through Celite® and the filtrate was concentrated in vacuo. The crude product was purified by flash silica gel chromatography (4:1 hexanes/EtOAc) to afford 62 mg (17%) of 18 together with 128 mg (43%) of unreacted 17. Mass spectrum, m/z=[426.1] (M+H)+.

Using the general procedures outlined in Schemes I through XIII and Scheme XVI and the appropriate amino acid analogues to the amino acid reagents Cbz-Hyp-OH, Boc-Thr(Me)-OH, and Boc-N(Me)Ala-OH the compound reported in Table 4 were prepared and tested for its binding affinities (Kd) to XIAP BIR-3 or cIAP-1 BIR-3.

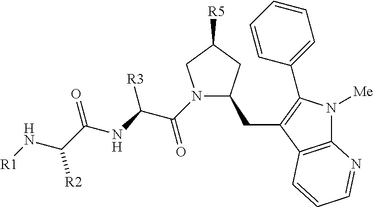

TABLE 4

| | | | | | | Observed |
|---|---|---|---|---|---|---|
| | | | | | Kd | Mass |
| Compound | R1 | R2 | R3 | R5 | (μM) | (m/z) |
| Y | Me | Me | (2R—EtOMe) | H | A | 492.6 |

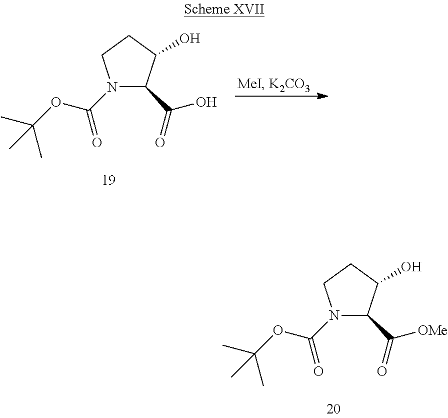

3-Hydroxypyrrolidine-1,2-dicarboxylic acid 1-tert-butyl ester 2-methyl ester (20): A solution containing 3-hydroxy-pyrrolidine-1,2-dicarboxylic acid 1-tert-butyl ester (19, 16 g, 71 mmol. See: Hodges, J. A.; Raines, R. T. J. Am. Chem. Soc. 2005, 45, 15923) in DMF (100 mL) was cooled to 0° C. To this solution was added K₂CO₃ (16 g, 116 mmol) followed by iodomethane (5.4 mL, 87 mmol). The reaction mixture was slowly warmed to ambient temperature over 1 h at which time it became a yellow heterogeneous solution. This mixture was heated at 90° C. for 1 h and then cooled to ambient temperature. The solution was diluted with brine, extracted with diethyl ether, dried over anhydrous Na₂SO₄, filtered, and concentrated to afford 14.8 g (87%) of 20 as a yellow oil (See: Demange, L.; Cluzeau, J.; Menez, A.; Dugave, C. Tetrahedron Lett. 2001, 42, 651).

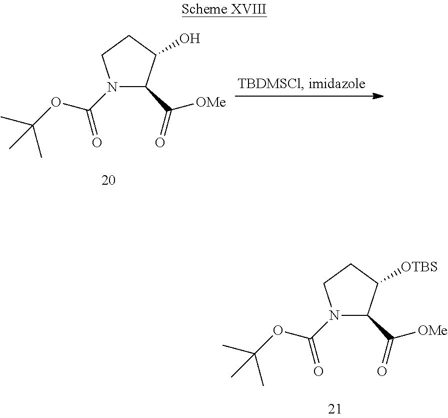

3-(tert-Butyldimethylsilanyloxy)pyrrolidine-1,2-dicarboxylic acid 1-tert-butyl ester 2-methyl ester (21): A solution containing alcohol 20 (14.8 g, 60 mmol) in DCM (150 mL) was cooled to 0° C. To this solution was added imidazole (5.4 g, 79 mmol) followed by t-butyl-dimethylsilyl-chloride (10 g, 66 mmol) in two portions. The reaction mixture was warmed to ambient temperature over 1 h. After 5 h, the solution was diluted with 1M HCl and extracted twice with DCM. The combined organic extracts were dried over anhydrous Na₂SO₄, filtered, and concentrated to afford 21.2 g (99%) of 21 as a yellow oil. ¹H NMR (CDCl₃, 300 MHz) δ 4.38-4.34 (m, 1H), 4.18 (br s, rotomers, 0.5H), 4.04 (app d, J=2.1 Hz, rotomers, 0.5H), 3.74 (s, 3H), 3.62-3.50 (m, 2H), 2.04-1.96 (m, 1H), 1.85-1.78 (m, 1H), 1.46 (s, minor rotomer), 1.41 (s, 9H), 0.92 (s, minor rotomer), 0.86 (s, 9H), 0.11 (s, 6H), 0.09 (s, minor rotomer) ppm.

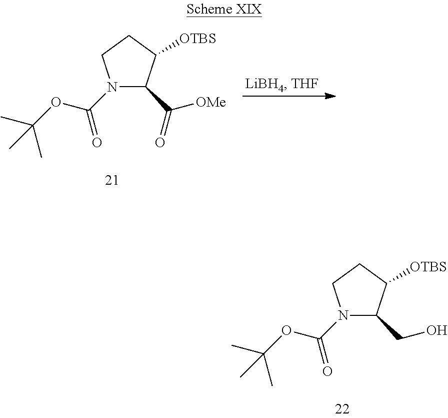

3-(tert-Butyldimethylsilanyloxy)-2-hydroxymethylpyrrolidine-1-carboxylic acid tert-butyl ester (22): A solution containing 21 (12 g, 33 mmol) in THF (50 mL) was cooled to 0° C. LiBH₄ in THF (2M, 20 mL) was added in a dropwise fashion. After 1 h, the solution was warmed to ambient temperature. After 2 h, the solution was diluted with MeOH, then H₂O, and concentrated. The residue was extracted with EtOAc, washed with 1M HCl, saturated aqueous NaHCO₃, brine, dried over anhydrous Na₂SO₄, filtered, and concentrated to afford 9.5 g (87%) of 22 as a colorless oil (See: Herdeis, C.; Hubmann, H. P.; Lotter, H. Tetrahedron: Asymmetry, 1994, 5, 119).

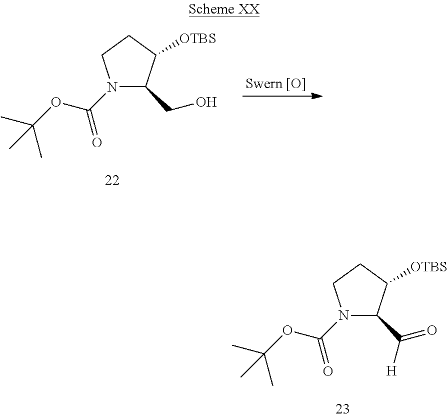

3-(tert-Butyldimethylsilanyloxy)-2-formylpyrrolidine-1-carboxylic acid tert-butyl ester (23): A solution containing 2M oxalyl chloride in DCM (22 mL) in DCM (40 mL) was cooled to −78° C. A solution containing DMSO (3.2 mL, 45 mmol) in DCM (20 mL) was added in a dropwise fashion. After 45 min, alcohol 22 (9.5 g, 29 mmol) in DCM (50 mL) was added in a dropwise fashion. After 45 min, TEA (16 mL, 115 mmol) was added in a dropwise fashion. The reaction mixture was warmed and maintained at 0° C. for 15 min. The solution was diluted with 1M HCl, extracted with DCM, washed with brine, dried over anhydrous Na₂SO₄, filtered, and concentrated to afford 9.5 g (100%) of 23 as a yellow oil. ¹H NMR (CDCl₃, 300 MHz) δ 9.53 (d, J=29 Hz, 1H), 4.39-4.36 (m, 1H), 4.24 (m, rotomer, 0.5H), 3.93 (m, rotomer, 0.5H), 3.73-3.49 (m, 2H), 1.98-1.86 (m, 2H), 1.47 (s, minor rotomer), 1.41 (s, 9H), 0.88 (s, 9H), 0.09 (s, 6H), 0.07 (s, minor rotomer) ppm.

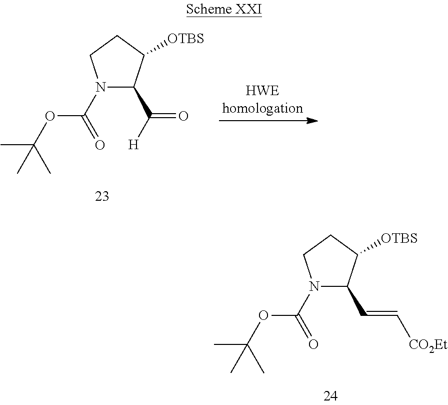

3-(tert-Butyldimethylsilanyloxy)-2-(2-ethoxycarbonylvinyl)pyrrolidine-1-carboxylic acid tert-butyl ester (24): To a suspension containing NaH (60%, 1.9 g, 46 mmol) in THF (50 mL) was slowly added triethylphosphonoacetate (7.5 mL, 38 mmol) in THF (20 mL) at 0° C. After 30 min, a solution containing aldehyde 23 (9.5 g, 29 mmol) in THF (40 mL) was then added in a dropwise fashion. The solution became orange-colored and stirring was continued for 0.5 h. The reaction mixture was diluted with brine, extracted with EtOAc, dried over anhydrous Na₂SO₄, filtered, and concentrated to afford 8.6 g (74%) of 24 as a yellow oil which was used without further purification. ¹H NMR (CDCl₃, 300 MHz) δ 6.82-6.72 (m, 1H), 5.87 (d, J=15.6 Hz, 1H), 4.24-4.11 (m, 4H), 3.67-3.46 (m, 2H), 1.94-1.89 (m, 1H), 1.79 (m, 1H), 1.48 (s, rotomer, 4.5H), 1.41 (s, rotomer, 4.5H), 1.31-1.24 (m, 3H), 0.91-0.88 (m, 9H), 0.09-0.07 (m, 6H) ppm.

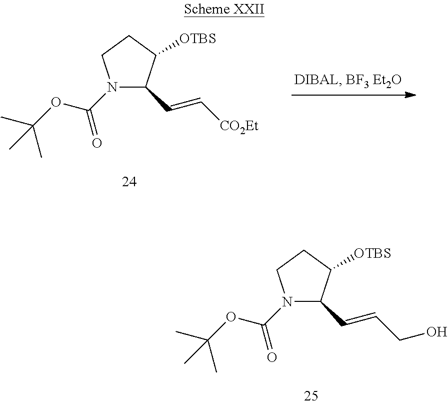

3-(tert-Butyldimethylsilanyloxy)-2-(3-hydroxypropenyl)pyrrolidine-1-carboxylic acid tert-butyl ester (25): A solution containing 24 (8.6 g, 22 mmol) in DCM (80 mL) was cooled to −78° C. To this solution was slowly added boron trifluoride etherate (2.8 mL, 22 mmol) followed by the addition of 1M DIBAL in DCM (60 mL). The solution was stirred at −78° C. for 1 h. The reaction mixture was then treated with EtOAc and stirred for 30 min. The reaction mixture was allowed to warm to −5° C. The reaction was quenched by the dropwise addition of 1M HCl. The mixture was diluted with DCM and H₂O and the layers were separated. The aqueous layer was extracted with DCM. The combined organic extracts were dried over anhydrous Na₂SO₄, filtered, and concentrated to afford 8.5 g of 25 as a light yellow oil which was used without further purification. ¹H NMR (CDCl₃, 300 MHz) δ 5.70 (m, 1H), 5.59-5.55 (m, 1H), 4.16-4.13 (m, 2H), 4.05 (m, 2H), 3.72-3.35 (m, 4H), 1.95-1.88 (m, 2H), 1.77-1.67 (m, 2H), 1.48-1.44 (m, 9H), 0.88 (s, 9H), 0.08-0.03 (m, 6H) ppm.

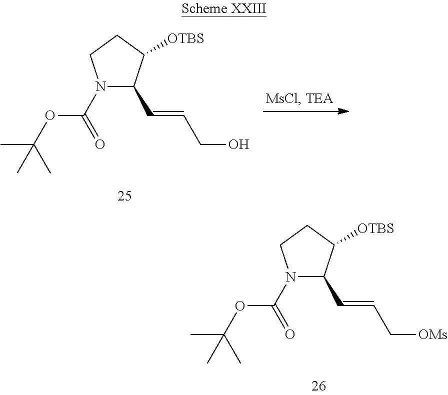

trans-2R-[3-(tert-Butyldimethylsilanyloxy)]-2-(3-methanesulfonyloxypropenyl)pyrrolidine-1-carboxylic acid tert-butyl ester (26): To a solution containing alcohol 25 (8.5 g, 24 mmol) in DCM (30 mL) was added triethylamine (4.0 mL, 29 mmol). The solution was cooled in an ice bath and methanesulfonyl chloride (2 mL, 26 mmol) was added in a dropwise fashion. The reaction mixture was stirred at ambient temperature for 30 min. Water (10 mL) was added and the product was extracted with DCM (3×50 mL). The organic extracts were combined and washed with 1M HCl, brine, dried over anhydrous Na₂SO₄, filtered, and concentrated to afford 8.9 g of 26 (92% over two steps) as an orange oil that was used without further purification. ¹H NMR (CDCl₃, 300 MHz) δ 5.73 (m, 1H), 4.71 (d, J=5.4 Hz, 1H), 4.30-4.15 (m, 1H), 4.06 (m, 1H), 3.54-3.33 (m, 2H), 3.02 (s, 3H), 1.94-1.89 (m, 1H), 1.79-1.78 (m, 1H), 1.45-1.43 (m, 9H), 0.92-0.87 (m, 9H), 0.09-0.07 (m, 6H) ppm.

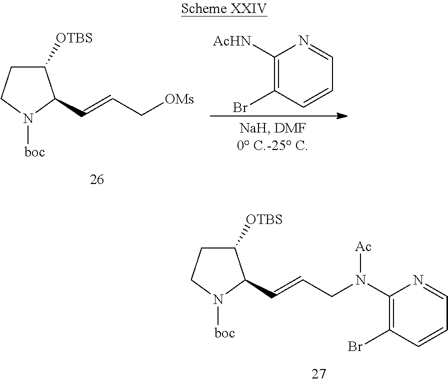

2-{3-[Acetyl-(3-bromo-pyridin-2-yl)-amino]-propenyl}-3-(tert-butyl-dimethyl-silanyloxy)-pyrrolidine-1-carboxylic acid tert-butyl ester (27): To a well-stirred solution of N-(3-Bromo-pyridin-2-yl)-acetamide (2.24 g, 10.4 mmol) in DMF (8 mL) at 0° C. was added NaH (522 mg, 13.0 mmol, 60% disp. in mineral oil) in one portion. Gas evolution was immediately noted. The solution was stirred at 0° C. for 30 minutes after which time it was warmed to room temperature and stirred for an additional 45 min. The reaction was recooled to 0° C. and a solution of 26 (4.31 g, 10.4 mmol) in DMF (12 mL) was added dropwise over 10 min. The reaction was stirred for an additional 4 hr warming gradually to room temperature. The reaction was quenched with brine, and extracted with EtOAc. The organic was washed with copious water and brine, dried over Na₂SO₄, filtered and concentrated. The crude residue was purified via flash chromatography (SiO₂, 1:1 EtOAc/hexanes) to afford 27 (2.53 g, 44%) as an orange oil. Mass spectrum, m/z=[556.0] (M)+.

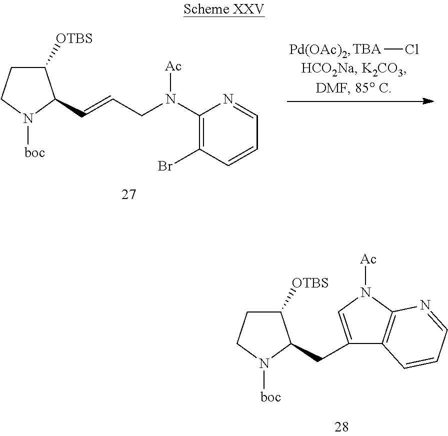

2-(1-Acetyl-1H-pyrrolo[2,3-b]pyridine-3-ylmethyl)-3-(tert-butyl-dimethyl-silanyloxy)-pyrrolidine-1-carboxylic acid tert-butyl ester (28): To a well-stirred solution of 27 (2.53 g, 4.56 mmol) in DMF (23 mL) was added tetra-n-butyl ammonium chloride (1.27 g, 4.56 mmol), Sodium Formate (310 mg, 4.56 mmol), and K₂CO₃ (818 mg, 5.93 mmol) and Pd(OAc)₂ (20 mg, 0.09 mmol). The resultant solution was heated to 85° C. for 2.5 hr., during which time the color changed from orange to black. The reaction was then cooled to room temperature, quenched with brine, and extracted with EtOAc. The organic phase was washed with water and brine, dried over Na₂SO₄, filtered and concentrated. The crude residue was purified via flash chromatography (SiO₂, 4:1 Hex/EtOAc) to afford 28 (1.32 g, 61%) as a colorless oil. Mass spectrum, m/z=[474.1] (M)+.

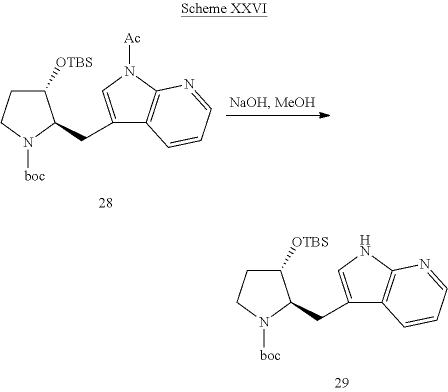

3-(tert-Butyl-dimethyl-silanyloxy)-2-(1H-pyrrolo[2,3-b]pyridine-3-ylmethyl)-pyrrolidine-1-carboxylic acid tert-butyl ester (29): To a well-stirred solution of 28 (1.32 g, 2.79 mmol) in MeOH (15 mL) was added 1 M NaOH (5 mL). The reaction was stirred for 30 minutes at room temperature, after which time it was concentrated. The residue was dissolved in CH₂Cl₂, washed with brine, dried over Na₂SO₄, filtered and concentrated to afford 29 (1.12 g, 93%) as a foamy white solid which was taken forward without further purification. Mass spectrum, m/z=[432.1] (M)+.

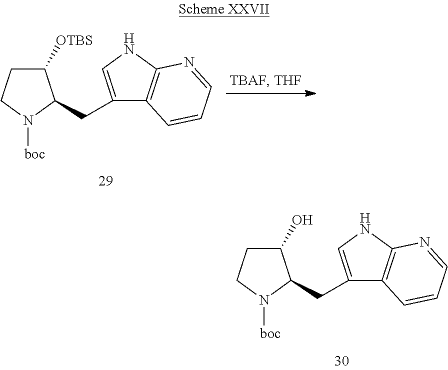

3-Hydroxy-2-(1H-pyrrolo[2,3-b]pyridin-3-ylmethyl)-pyrrolidine-1-carboxylic acid tert-butyl ester (30): To a well-stirred solution of 29 (1.12 g, 2.59 mmol) in THF (13 mL) at room temperature was added a 1.0 M solution of TBAF in THF (3.9 mL, 3.9 mmol). The reaction was stirred overnight, after which time the reaction was concentrated and the residue purified directly via flash chromatography (SiO₂, 100% EtOAc) to afford 30 (730 mg, 89%) as a foamy white solid. Mass spectrum, m/z=[318.4] (M)+.

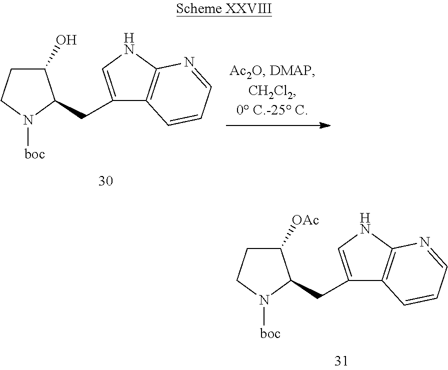

3-Acetoxy-2-(1H-pyrrolo[2,3-b]pyridine-3-ylmethyl)-pyrrolidine-1-carboxylic acid tert-butyl ester (31): To a well-stirred solution of 30 (620 mg, 1.95 mmol) in CH₂Cl₂ (10 mL) at ° C. was added DMAP (cat.) followed by Ac₂O (184 uL, 1.95 mmol). The reaction was continued stirring overnight warming gradually to room temperature. The reaction was concentrated and the residue purified directly via flash chromatography (SiO₂, 1:1 EtOAc/Hex) to afford 31 (690 mg, 98%) as a white foamy solid. Mass spectrum, m/z=[360.0] (M)+.

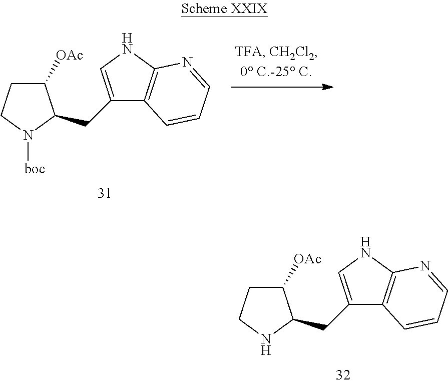

Acetic acid 2-(1H-pyrrolo[2,3-b]pyridin-3-ylmethyl)-pyrrolidin-3-yl ester (32): To a well-stirred solution of 31 (726 mg, 2.02 mmol) in CH₂Cl₂ (8 mL) at 0° C. was added TFA (2 mL). The reaction was stirred for an additional 5 h. The reaction was concentrated and the crude residue was taken up in 10% MeOH/CH₂Cl₂, washed with NaHCO₃ (sat) and brine and concentrated. The residue was then taken up in MeOH, filtered and concentrated to afford 32 (485 mg, 93%) as a white solid. Mass spectrum, m/z=[260.0] (M)+.

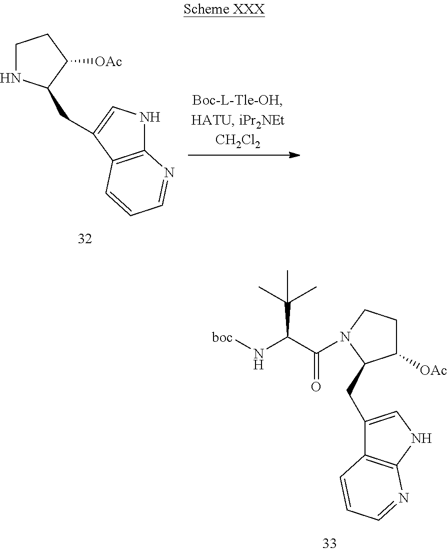

Acetic acid 1-(2-tert-butoxycarbonylamino-3,3-dimethyl-butyryl)-2-(1H-pyrrolo[2,3-b]pyridin-3-ylmethyl)-pyrrolidin-3-yl ester (33): To a well-stirred solution of Boc-Tle-OH (206 mg, 0.89 mmol) in DMF (1 mL) at 0° C. was added iPr₂NEt (220 uL, 1.28 mmol) and HATU (339 mg, 0.89 mmol). The resultant pale yellow solution was allowed to stir for an additional 20 min at 0° C. after which time a solution of 32 (220 mg, 0.85 mmol) in DMF (2 mL) was added. The reaction was stirred overnight while warming gradually to room temperature. The reaction was diluted with EtOAc, washed with water and brine, dried over Na₂SO₄, filtered and concentrated. The resultant crude was purified via flash chromatography (SiO₂, gradient 1:1 EtOAc/Hex to 100% EtOAc) to afford 33 (390 mg, 97%) as an off-white solid. Mass spectrum, m/z=[473.1] (M)+.

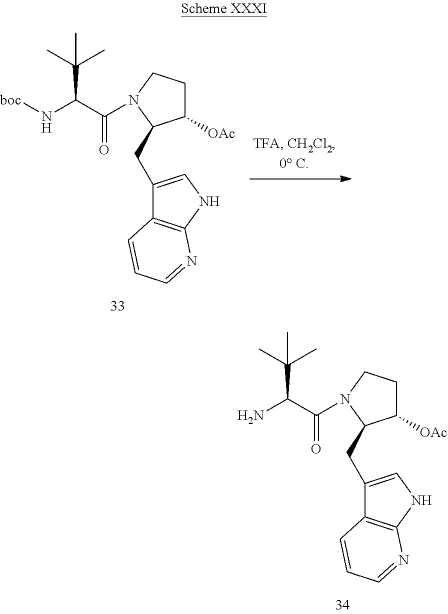

Acetic acid 1-(2-amino-3,3-dimethyl-butyryl)-2-(1H-pyrrolo[2,3-b]pyridin-3-ylmethyl)-pyrrolidin-3-yl ester (34): To a well-stirred solution of 33 (390 mg, 0.83 mmol) in CH₂Cl₂ (8 mL) at 0° C. was added TFA (2 mL). The reaction was stirred for 20 min at 0° C. then warmed to room temperature for an additional 2 h. The reaction mixture was then concentrated and the residue dissolved in 10% MeOH/CH₂Cl₂, washed with NaHCO₃ (sat) and brine, and concentrated. The residue was then dissolved in CH₂Cl₂, dried over Na₂SO₄, filtered and concentrated to afford 34 (255 mg, 83%) as a brown-colored foam which was taken forward without further purification. Mass spectrum, m/z=[373.1] (M)+.

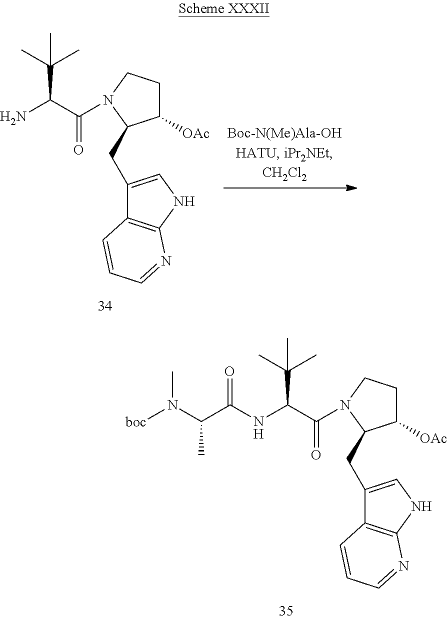

Acetic acid 1-{2-[2-(tert-butoxycarbonyl-methyl-amino)-propionylamino]-3,3-dimethyl-butyryl}-2-(1H-pyrrolo[2,3-b]pyridin-3-ylmethyl)-pyrrolidin-3-yl ester (35): To a well-stirred solution of Boc-N(Me)Ala-OH (72 mg, 0.35 mmol) in DMF (1 mL) at 0° C. was added iPr₂NEt (90 uL, 0.35 mmol) and HATU (133 mg). The reaction was continued stirring for 20 min, after which time a solution of 34 (125 mg, 0.34 mmol) in DMF (2 mL) was added. The reaction was allowed to stir overnight warming gradually to room temperature. The reaction was then diluted with EtOAc, washed with water and brine, dried over Na₂SO₄, filtered and concentrated to afford 35 (150 mg, 79%) as an off-white solid that was taken forward without further purification. Mass spectrum, m/z=[558.2] (M)+.

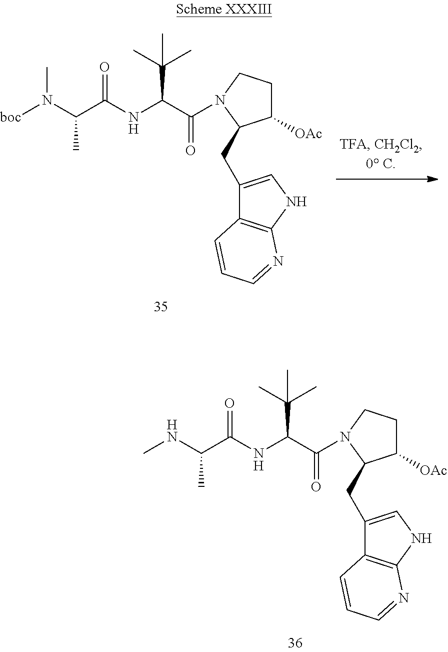

Acetic acid 1-[3,3-dimethyl-2-(2-methylamino-propionylamino)-butyryl]-2-(1H-pyrrolo[2,3-b]pyridin-3-ylmethyl)-pyrrolidin-3-yl ester (36): To a well-stirred solution of 35 (150 mg, 0.27 mmol) in CH₂Cl₂ (6 mL) at 0° C. was added TFA (1 mL) and the reaction was stirred at 0° C. for 1 h, then warmed to room temperature for 1 h. The reaction mixture was concentrated and the residue dissolved in 10% MeOH/CH₂Cl₂, washed with NaHCO₃ (sat.) and brine, and concentrated. The residue was then taken up in MeOH, filtered and concentrated to afford 36 (128 mg, >100%) as a yellowish oil that was taken forward without further purification. Mass spectrum, m/z=[458.2] (M)+.

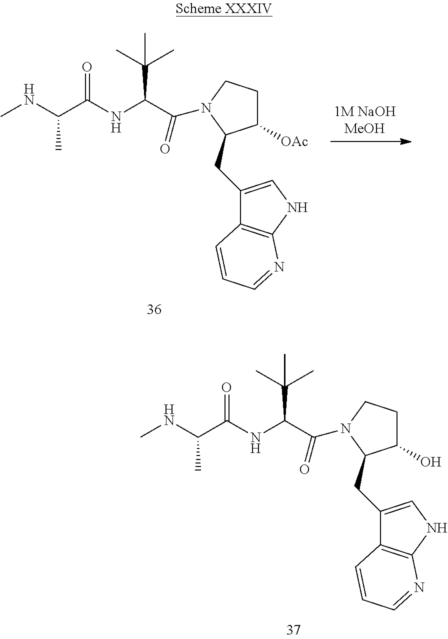

N-{1-[3-Hydroxy-2-(1H-pyrrolo[2,3-b]pyridin-3-ylmethyl)-pyrrolidine-1-carbonyl]-2,2-dimethyl-propyl}-2-methylamino-propionamide (37): To a well-stirred solution of 36 (128 mg, 0.28 mmol) in MeOH (3 mL) at 0° C. was added 1 M NaOH (1 mL). The reaction was stirred for 1.5 h then concentrated. The residue was purified directly via reverse phase HPLC (C18, 10-70% MeCN/H₂O, 30 min). The appropriate fractions were collected and lyophilized to afford 37 (69 mg, 59%) as a flocculent white solid. ¹³C NMR (75 MHz, CDCl₃) δ 173.5, 173.3, 170.5, 170.2, 148.0, 142.3, 128.3, 127.9, 124.2, 123.5, 120.7, 120.6, 115.7, 115.4, 110.7, 109.9, 74.1, 72.1, 68.0, 66.8, 59.2, 58.8, 57.3, 46.2, 44.4, 36.2, 35.5, 33.7, 33.3, 31.6, 29.7, 28.0, 26.6, 22.3, 18.6, 18.2 ppm. Mass spectrum, m/z=[415.2] (M)+.

Using the general procedures outlined in Schemes XVII through XXXIV and the appropriate amino acid analogues to the amino acid reagents Boc-Tle-OH and Boc-N(Me)Ala-OH the compounds reported in Table 5 were prepared and tested for their binding affinities (Kd) to XIAP BIR-3 or cIAP-1 BIR-3.

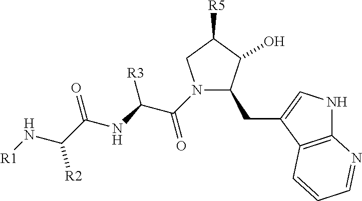

TABLE 5

| | | | | | | Observed |
|---|---|---|---|---|---|---|
| | | | | | | Mass |
| Compound | R1 | R2 | R3 | R5 | Kd (μM) | (m/z) |
| Z | Me | Me | tert-Butyl | H | A (cIAP-1) | 416.2 |
| AA | Et | Me | tert-Butyl | H | A (cIAP-1) | 430.2 |
| BB | Me | Me | (2R—EtOMe) | H | A (cIAP-1) | 418.2 |
| CC | Et | Me | (2R—EtOMe) | H | A (cIAP-1) | 432.2 |
| DD | Me | Me | iPr | H | A (cIAP-1) | 402.2 |
| EE | Et | Me | iPr | H | A (cIAP-1) | 416.2 |

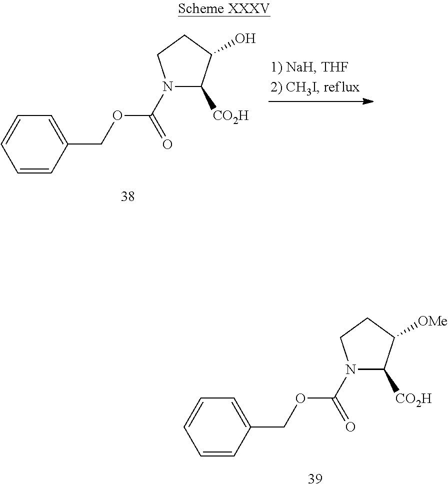

3-Methoxy-pyrrolidine-1,2-dicarboxylic acid 1-benzyl ester (39): To a solution of N-Cbz-3-hydroxyproline (38, 14.4 g, 54.5 mmol) in THF (180 mL) at room temperature was added NaH (7.6 g, 190.7 mmol) in three portions, during which time a slight exotherm and gas evolution was noted. After 1 h, CH₃I (13.3 mL, 109.0 mmol) was added and the reaction was heated to reflux. After 4 h, the yellow-colored reaction mixture was cooled to room temperature and allowed to stir overnight. The reaction mixture was concentrated and the residue was dissolved in EtOAc and extracted with H₂O. The bright yellow aqueous layer was acidified to pH 2 using 3M HCl and extracted with EtOAc. This yellow organic layer was washed with brine, dried over anhydrous Na₂SO₄, filtered and concentrated to afford 39 (13.4 g, 88%) as a viscous orange-colored oil which was used without further purification. Mass spectrum, m/z=[279.9] (M)+.

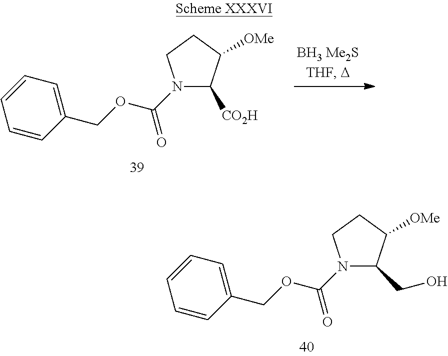

2-Hydroxymethyl-3-methoxy-pyrrolidine-1-carboxylic acid benzyl ester (40): To a solution of 39 (13.4 g, 48.1 mmol) in THF (160 mL) at room temperature was added a 2M solution of BH₃.DMS in THF (125 mL, 250.2 mmol) in one portion, during which time some bubbling was noted. The resultant pale solution was then heated at reflux. After 3 h, the reaction mixture was cooled to 0° C. and quenched by the dropwise addition of MeOH, during which time vigorous gas evolution was noted. The reaction mixture was concentrated and the resultant residue was taken up in EtOAc and washed successively with H₂O and brine. The combined aqueous phase was back-extracted with EtOAc, and the combined organic extracts were dried over anhydrous Na₂SO₄, filtered and concentrated. The crude product was purified by flash silica gel chromatography (1:1 EtOAc/hexanes) to afford 10.5 g (83%) of 40.

Using the general procedures outlined in Schemes XX through XXXVI and the appropriate amino acid analogues to the amino acid reagents Boc-Tle-OH and Boc-N(Me)Ala-OH the compounds reported in Table 6 were prepared and tested for their binding affinities (Kd) to XIAP BIR-3 or cIAP-1 BIR-3.

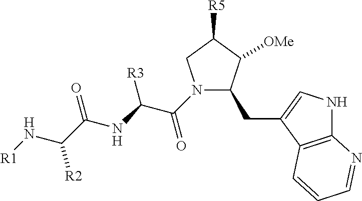

TABLE 6

| | | | | | | Observed |
|---|---|---|---|---|---|---|
| | | | | | Kd | Mass |
| Compound | R1 | R2 | R3 | R5 | (μM) | (m/z) |
| FF | Me | Me | tert-Butyl | H | C | 429.6 |
| GG | Et | Me | tert-Butyl | H | C | 443.7 |
| HH | Et | Me | (2R—EtOMe) | H | C | 445.3 |
| II | Et | Me | iPr | H | C | 429.7 |

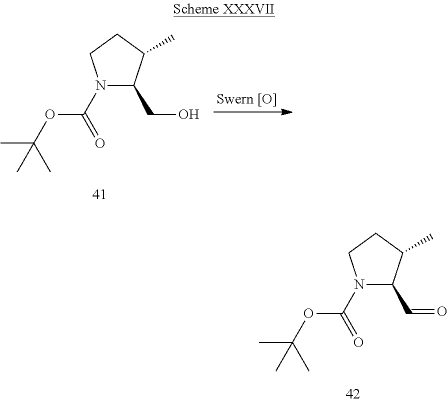

2-Formyl-3-methyl-pyrrolidine-1-carboxylic acid tert-butyl ester (42): A 500-mL three-necked flask equipped with an overhead stirrer and nitrogen inlet was charged with a 1M solution of oxalyl chloride in DCM (20.5 mL, 0.041 mol) and anhydrous DCM (100 mL) and cooled to −78° C. A solution of anhydrous DMSO (3.45 mL, 0.044 mol) in DCM (20 mL) was added dropwise with stirring. After 30 min, alcohol 41 (7.35 g, 0.034 mol. See: Herdeis, C.; Hubmann, H. P. Tetrahedron Asymmetry 1992, 3, 1213-1221; and, Ohfune, Y.; Tomita, M. J. Am. Chem. Soc. 1982, 104, 3511-3513) was added in DCM (40 mL) in a dropwise fashion. After 30 min, Et₃N (23.7 mL, 0.17 mol) was added resulting in the formation of a white suspension. The reaction mixture was transferred to a 0° C. ice/water bath and maintained for 30 min. The reaction mixture was quenched by the addition of water. The product was extracted with DCM and the combined organic extracts were washed successively with water, 1M HCl, and brine. The organic phase was dried over anhydrous Na₂SO₄, filtered, and concentrated to afford 7.05 g (99%) of aldehyde 42 which was used without further purification. ¹H NMR (CDCl₃, 300 MHz) δ 9.45 (s, minor rotamer), 9.40 (s, 1H, major rotamer), 3.78-3.35 (m, 3H), 2.3-2.0 (m, 2H), 1.70-1.55 (m, 1H), 1.47 (s, minor rotamer), 1.42 (s, 9H, major rotamer), 1.15 (d, J=6 Hz, 3H) ppm.

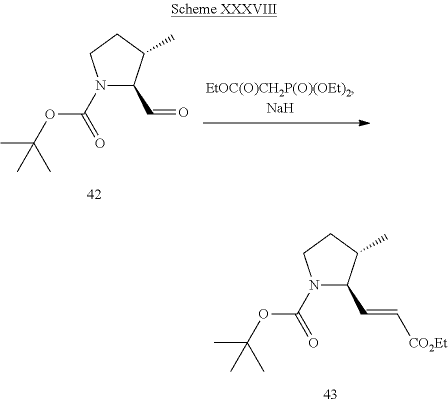

2-(2-Ethoxycarbonyl-ethyl-3-methyl-pyrrolidine-1-carboxylic acid tert-butyl ester (43): A 500-mL 3-neck round-bottomed flask was charged with sodium hydride (60%, 1.77 g, 0.044 mol) in anhydrous THF (100 mL) under nitrogen and cooled to 10° C. A solution of triethyl phosphono acetate (9.15 g, 0.041 mol) in THF (50 mL) was added drop wise to the NaH/THF suspension. Following the addition, crude aldehyde 42 (7.25 g, 0.034 mol) in THF (15 mL) was added in a dropwise fashion. After 1 h, the reaction was complete by TLC analysis [30% EtOAc/Hexanes: R_{f}(42)=0.7; R_{f}(43)=0.75]. The reaction mixture was quenched by the addition of saturated aqueous NH₄Cl. The product was extracted with EtOAc, washed with 1M HCl, water, brine, dried over anhydrous Na₂SO₄, filtered, and concentrated to afford 13.3 g of crude 43 (quant.) which was used without further purification. ¹H NMR (CDCl₃, 300 MHz) δ 6.8 (m, 1H), 5.82 (m, 1H), 4.2 (m, 2H), 4.0-3.25 (m, 3H), 2.2-1.85 (m, 2H), 1.70-1.55 (m, 1H), 1.47 (s, minor rotamer), 1.42 (s, 9H, major rotamer), 1.15 (d, J=6 Hz, 3H) ppm.

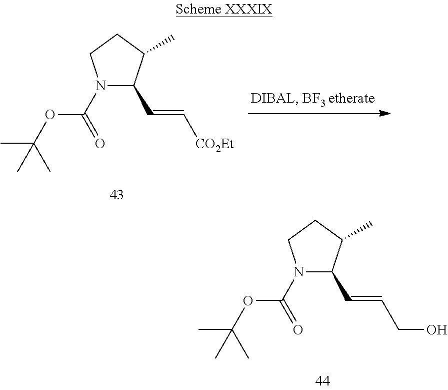

2-(3-hydroxy-propenyl)-3-methyl-pyrrolidine-1-carboxylic acid tert-butyl ester (44): A solution containing crude 43 (16.7 g, 0.059 mol) in DCM (150 mL) was cooled to −78° C. BF₃.Et₂O (8.9 mL, 0.07 mol) was added followed by the dropwise addition of DIBAL (2 M/DCM, 200 mL, 0.4 mol). After 2 h, TLC analysis indicated complete consumption of the 43 [TLC analysis: 1:1 hexane/EtOAc, R_{f}(44)=0.3]. EtOAc (40 mL) was added and the reaction mixture was warmed to −15° C. The reaction mixture was carefully quenched with 1M HCl until pH=2. The product was extracted with DCM. The organic extracts were washed with 1M HCl, water, and brine, dried over anhydrous Na₂SO₄, filtered, and concentrated. The crude product was purified by silica gel chromatography (2:1 hexanes/EtOAc) to afford 7.2 g (51%) of 44. ¹H NMR (CDCl₃, 300 MHz) δ 5.8-5.5 (m, 2H), 4.18 (m, 2H), 4.0-3.25 (m, 3H), 2.2-1.85 (m, 2H), 1.55-1.3 (m, 1H), 1.43 (s, 9H), 1.15 (d, J=6 Hz, 3H) ppm.

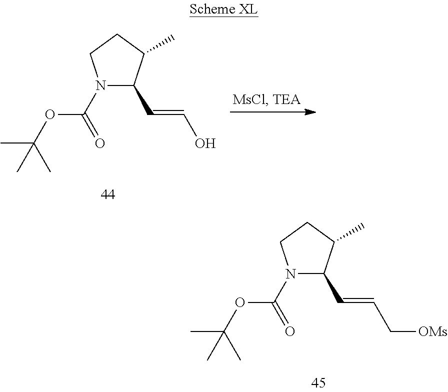

2-(3-Methanesulfonyloxy-propenyl)-3-methylpyrrolidine-1-carboxylic acid tert-butyl ester (45): To a solution containing 44 (6.0 g, 0.025 mol) in DCM (25 mL) at 0° C. was added Et₃N (4.5 mL, 0.032 mol). After 5 min, a solution containing methanesulfonylchloride (2.33 mL, 0.03 mol) in DCM (5 mL) was added dropwise. After 2 h, TLC analysis revealed complete consumption of 44 [1:1 hexanes/EtOAc, R_{f}(45)=0.5; R_{f}(44)=0.4]. The reaction mixture was poured onto ice-water and extracted with DCM. The organic extracts were washed with water, brine, and dried over anhydrous Na₂SO₄, filtered, and concentrated to afford 7.05 g (89%) of crude 45 as a pale brown oil which was used without further purification. ¹H NMR (CDCl₃, 300 MHz) δ 5.8-5.5 (m, 2H), 4.69 (d, J=6.15 Hz, 2H), 3.85-3.3 (m, 3H), 3.0 (s, 3H), 2.0-1.9 (m, 1H), 1.55-1.30 (m, 1H), 1.40 (s, 9H), 1.0 (d, J=6.74 Hz, 3H) ppm.

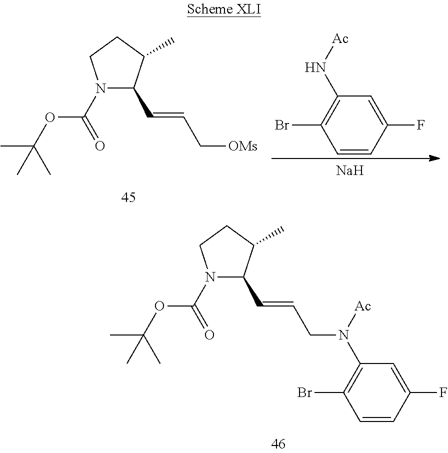

2-{3-[Acetyl-(2-bromo-5-fluoro-phenyl)-amino]-propenyl}-3-methyl-pyrrolidine-1-carboxylic acid (46): To a suspension of NaH (60%, 1.44 g, 0.036 mol) in DMF (15 mL) at 0° C. was added a solution containing 2-bromo-5-fluoroacetanilide (8.35 g, 0.036 mol) in DMF (10 mL). After 30 min, a solution containing crude 45 (9.58 g, 0.03 mol) in DMF (10 mL) was added and the reaction mixture was warmed to ambient temperature overnight. The reaction was quenched by pouring onto the ice-water containing 1M HCl. The product was extracted with diethyl ether, washed with water, brine, dried over anhydrous Na₂SO₄, filtered, and concentrated. The product was purified by flash silica gel chromatography (2:1 hexane/EtOAc) to afford 5.41 g (45%) of 46 as a pale brown viscous oil. ¹H NMR (CDCl₃, 300 MHz) δ 7.62 (m, 1H), 7.05 (m, 2H), 5.65-5.25 (m, 2H), 4.9-4.7 (m, 1H), 4.3-4.1 (m, 1H), 3.85-3.3 (m, 4H), 2-1.9 (m, 1H), 1.8 (s, 3H) 1.55-1.3 (m, 1H), 1.43 (s, 9H), 0.96 (d, J=6.15 Hz, 3H) ppm. Mass spectrum, m/z=[354.3] (M-Boc)+.

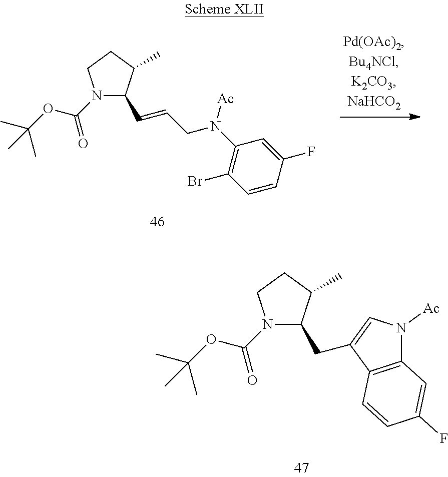

2-(1-Acetyl-6-fluoro-1H-indol-3-ylmethyl)-3-methyl-pyrrolidine-1-carboxylic acid tert-butyl ester (47): A solution containing 46 (5 g, 0.011 mol), n-Bu₄NCl (3.3 g, 0.012 mol), K₂CO₃ (1.65 g, 0.012 mol), and NaHCO₂ (0.81 g, 0.012 mol) in DMF (20 mL) was degassed under high vacuum. Palladium acetate (0.49 g, 0.002 mol) was added and the heterogeneous reaction mixture was immersed in a preheated (80-85° C.) oil bath. After 3 h, TLC analysis revealed complete consumption of 46 [1:1 hexane/EtOAc, R_{f}(46)=0.4, R_{f}(47)=0.5]. The reaction mixture was cooled in an ice bath and diethyl ether (100 mL) was added. The mixture was filtered through Celite® and the solids were washed with diethyl ether. The filtrate was washed with water, brine, dried over anhydrous Na₂SO₄, filtered, and concentrated. The crude product was purified by normal phase HPLC (10-100% EtOAc/hexane over 50 min) to afford 2.2 g (54%) of 47 as brown, viscous oil. ¹H NMR (CDCl₃, 300 MHz) δ 8.22-8.1 (m, 1H), 7.7-7.5 (m, 1H), 7.15-6.97 (m, 2H), 3.8-2.65 (m, 4H), 2.6 (s, 3H), 2.12-1.85 (m, 1H), 1.62 (s, 1H), 1.42 (s, 9H, major rotamer), 1.4 (s, minor rotamer), 0.9 (d, J=6 Hz, 3H) ppm. Mass spectrum, m/z=[274.5] (M-Boc)+.

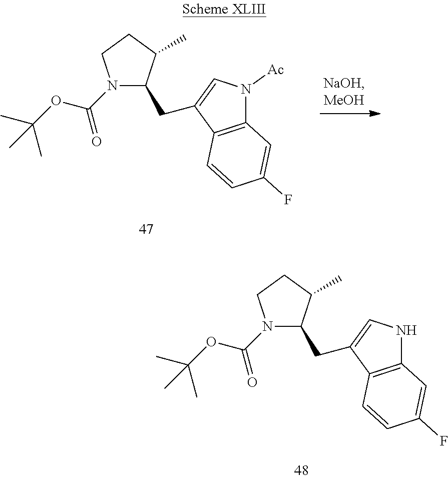

2-{6-Fluoro-1H-indol-3-ylmethyl)-3-methyl-pyrrolidin-1-carboxylic acid tert-butyl ester (48): To a solution containing 47 (2.2 g, 0.006 mol) in MeOH (15 mL) was added 1M NaOH (6 mL, 0.006 mol) at 0° C. After 30 min, TLC analysis revealed complete consumption of 47 [EtOAc/hexanes 1:1, R_{f}(47)=0.6; R_{f}(48)=0.5]. The solvent was removed in vacuo and the residue was dissolved in EtOAc. The organic phase was washed with 1M HCl, water, brine, dried over anhydrous Na₂SO₄, filtered, and concentrated to afford 2.11 g (quant.) of crude 48 which was used in the next step without further purification. ¹H NMR (CDCl₃, 300 MHz) δ 9.0 (s, 1H, major rotamer), 8.85 (s, minor rotamer), 7.62-7.5 (m, 1H), 7.1-6.72 (m, 3H), 3.8-2.7 (m, 5H), 2.15-1.3 (m, 3H), 1.55 (s, 9H), 0.85 (d, J=7 Hz, 3H) ppm.

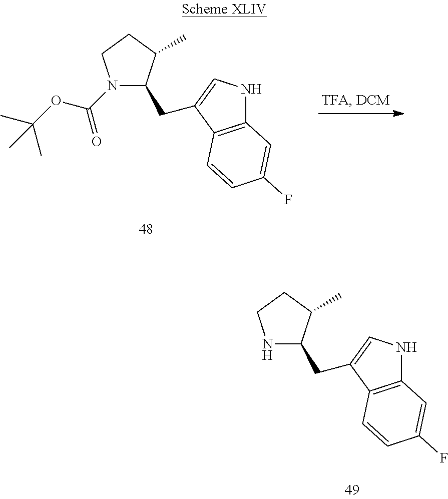

6-Fluoro-3-(3-methyl-pyrrolidin-2-ylmethyl)-1H-indole (49): To solution containing 48 (0.89 g, 0.0024 mol) in DCM (20 mL) at 0° C. was added TFA (4 mL). After 2 h, TLC analysis revealed complete consumption of 48 [10% MeOH/DCM, R_{f}(48)=0.7, R_{f}(49)=0.3]. The reaction mixture was concentrated in vacuo, diluted with DCM, washed with aqueous NaHCO₃, brine, dried over anhydrous Na₂SO₄, filtered, and concentrated to afford 0.6 g (86%) of 49 which was used without further purification. ¹H NMR (CDCl₃, 300 MHz) δ 9.0 (br s, 1H), 7.6-7.35 (m, 1H), 7.1-6.7 (m, 3H), 4.2 (br m, 1H), 3.2-2.5 (m, 5H), 2.1-1.2 (m, 3H), 1.05 (d, J=6.74 Hz, 3H) ppm.

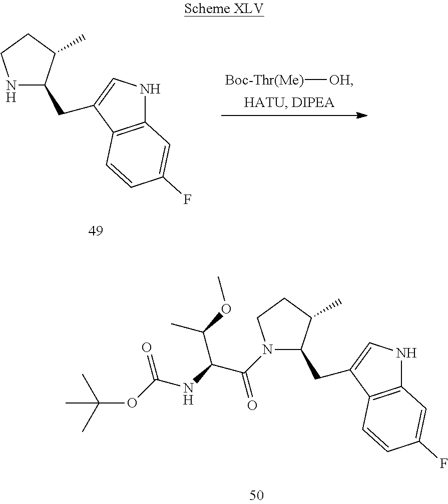

{1-[2-(6-Fluoro-1H-indol-3-ylmethylpyrrolidine-1-carbonyl]-2-methoxy-propyl}carbamic acid tert-butyl ester (50): To a solution containing crude 49 (0.3 g, 1.1 mmol) and Boc-Thr(Me)-OH (0.31 g, 1.3 mmol) in NMP (5 mL) at 0° C. was added DIPEA (0.25 mL, 1.44 mmol) followed by HATU (0.5 g, 1.3 mmol) and the reaction mixture was stirred at ambient temperature for 6 h. The reaction mixture was diluted with EtOAc and washed successively with dilute aqueous HCl, water, saturated aqueous NaHCO₃, water, and brine. The organic phase was dried over anhydrous Na₂SO₄, filtered, and concentrated. The product was purified by reverse-phase HPLC (C18; 50-100% ACN/water v/v 0.1% AcOH). The product-containing fractions were concentrated in vacuo to afford 0.28 g (48%) of 50 as a white solid. ¹H NMR (CDCl₃, 300 MHz): δ 8.2 (s, 1H), 7.8-7.5 (m, 1H), 7.05 (m, 2H), 6.92 (m, 1H), 5.6 (d, J=10.7 Hz, 1H), 4.6 (m, 1H), 4.1 (m, 1H), 3.6 (m, 3H), 3.4 (s, 3H), 3.35 (m, 1H), 2.6 (m, 1H), 2.1 (m, 2H), 1.7 (m, 1H), 1.48 (m, H) 1.45 (s, 9H), 1.21 (d, J=6.45 Hz, 3H, major rotamer), 1.14 (d, J=6.45 Hz, minor rotamer), 0.90 (d, J=7.03 Hz, minor rotamer), 0.76 (d, J=6.45 Hz, 3H, major rotamer) ppm. Mass spectrum, m/z=[447.7] (M)+.

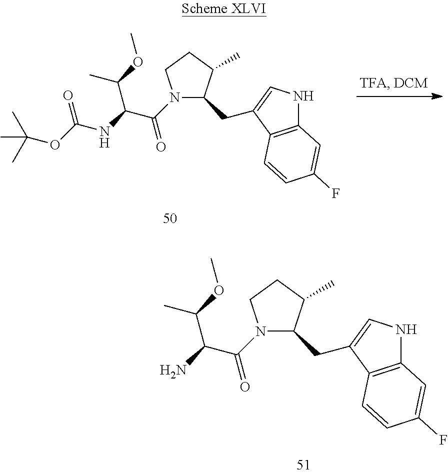

2-Amino-1-[2-(6-fluoro-1H-indol-3-ylmethyl)-3-methylpyrrolidin-1-yl]-3-methoxy-butan-one (51): To a solution containing 50 (0.28 g, 0.63 mmol) in DCM (20 mL) at 0° C. was added TFA (4 mL). After 2 h, TLC analysis showed the complete consumption of 50 [10% MeOH/DCM, R_{f}(50)=0.6, R_{f}(51)=0.2]. The reaction mixture was concentrated in vacuo and the residue was dissolved in DCM and washed successively with saturated aqueous NaHCO₃, and brine. The organic extracts were dried over anhydrous Na₂SO₄, filtered, and concentrated to afford 0.36 g (quant.) of crude 51 which was used without further purification.

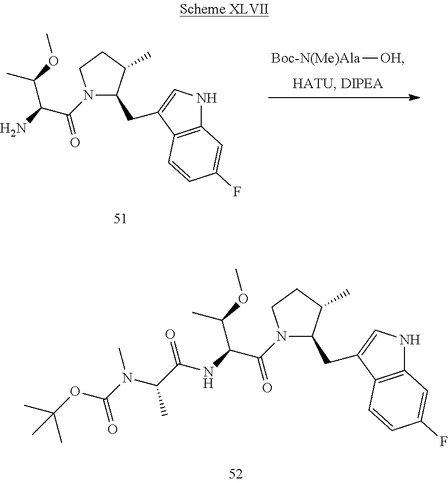

{1-{1-2-(6-fluoro-1H-indol-3-ylmethyl)-3-methyl-pyrrolidine-1-carbonyl]-2-methoxypropylcarbamoyl}-ethyl}-methyl-carbamic acid tert-butyl ester (52): To a solution containing 51 (0.09 g, 0.26 mmol) and Boc-N(Me)Ala-OH (0.063 g, 0.31 mmol) in NMP (3 mL) at 0° C. was added DIPEA (0.075 mL, 0.43 mmol) followed by HATU (0.13 g, 0.34 mmol). The reaction mixture was stirred at ambient temperature for 16 h. The reaction mixture was diluted with EtOAc and then washed with 1M HCl, saturated aqueous NaHCO₃, water, and brine. The organic extract was dried over anhydrous Na₂SO₄, filtered, and concentrated. The crude product was purified by RP HPLC (C18; 50-100% ACN/water v/v 0.1% HOAc) to afford 0.052 g (38%) of 52. ¹H NMR (CDCl₃, 300 MHz), ˜0.3:1 mixture of rotamers: δ 9.4 (s, minor rotamer), 8.9 (s, 1H, major rotamer), 7.76-7.4 (m, 1H), 7.05-6.89 (m, 2H), 6.9-6.82 (m, 1H), 4.08-3.95 (m, 2H), 3.7-3.2 (m, 5H), 3.38 (s, 3H), 3.35-3.25 (m, 1H), 2.8 (s, 3H), 2.65-2.55 (m, 1H), 2.3-2.0 (m, 1H). 1.5 (s, 9H), 1.34 (d, J=7.3 Hz, 3H, major rotamer), 1.29 (d, J=7.3 Hz, minor rotamer), 1.17 (d, J=6.4 Hz, 3H, major rotamer), 0.91 (d, J=7.0 Hz, minor rotamer), 0.73 (d, J=6.7 Hz, 3H, major rotamer) ppm. Mass spectrum, m/z=[532.8] (M)+.

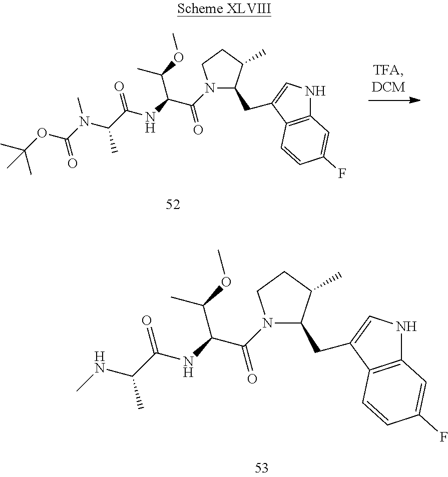

N-{1-{1-2-(6-fluoro-1H-indol-3-ylmethyl)-3-methyl-pyrrolidine-1-carbonyl]-2-methoxy-propyl}-2-methylamino-propionamide (53): To a solution containing 52 (0.052 g, 0.1 mmol) in DCM (20 mL) at 0° C. was added TFA (4 mL). After 1 h, TLC and mass spectrum analysis revealed the completion consumption of 52 [10% MeOH/DCM, R_{f}(52)=0.6, R_{f}(53)=0.2]. The reaction mixture was concentrated in vacuo and the residue was neutralized by the addition of saturated aqueous NaHCO₃. The aqueous solution was purified by reverse-phase HPLC (water/ACN v/v 0.1% HOAc) to afford pure acid addition salt 53.HOAc (0.058 g). ¹H NMR (CDCl₃, 300 MHz): δ 9.2 (s, 1H), 8.2 (s, 0.5H), 7.8 (d, J=8 Hz, 1H), 7.6 (m, 1H), 7.05-7.02 (m, 2H), 6.92-6.80 (m, 1H), 4.84-4.8 (m, 1H), 4.15-4.03 (m, 1H), 3.83-3.75 (m, 1H), 3.72-3.63 (m, 1H), 3.58-3.5 (m, 2H), 3.39 (s, 3H), 3.32-3.26 (m, 1H), 2.9-2.65 (m, 1H), 2.58 (s, 3H), 2.48-2.1 (m, 2H), 1.57-1.5 (m, 1H), 1.46 (d, J=7 Hz, 3H), 1.2 (d, J=6 Hz, 3H), 0.75 (d, J=6 Hz, 3H) ppm. Mass spectrum, m/z=[432.7] (M)+.

Using the general procedures outlined in Schemes XXXVII through XLVIII and using the appropriate amino acid analogues to the amino acid reagents Boc-Thr(Me)-OH and Boc-N(Me)Ala-OH, the compounds reported in Table 7 were prepared and tested for their binding affinities (Kd) to XIAP BIR-3 or cIAP-1 BIR-3.

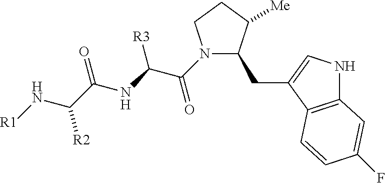

TABLE 7

| | | | | | Observed |
|---|---|---|---|---|---|
| Compound | R1 | R2 | R3 | Kd (μM) | Mass (m/z) |
| JJ | Me | Me | (2R—EtOMe) | B | 432.7 |
| KK | Me | Et | (2R—EtOMe) | D | 446.7 |
| LL | Me | CH₂OH | (2R—EtOMe) | D | 448.7 |
| MM | Et | Me | (2R—EtOMe) | C | 446.7 |
| NN | Me | Me | (2R—EtOH) | C | 419.3 |
| OO | Me | Et | (2R—EtOH) | B | 433.3 |
| PP | Me | CH₂OH | (2R—EtOH) | D | 435.3 |
| QQ | Et | Me | (2R—EtOH) | D | 433.3 |

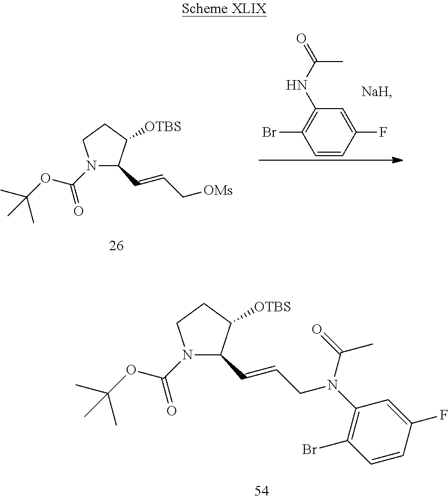

trans-2R-[2-{3-[Acetyl-(2-bromo-5-fluorophenyl)amino]propenyl}]-3-(tert-butyldimethylsilanyloxy)pyrrolidine-1-carboxylic acid tert-butyl ester (54): To a solution containing N-(2-bromo-5-fluorophenyl)acetamide (5.7 g, 24 mmol) in DMF (30 mL) was added NaH (60%, 1.2 g, 30 mmol) at 0° C. After 30 min, the solution was warmed and maintained at ambient temperature for 30 min. To this solution was slowly added mesylate 26 (See Scheme XXIII) (8.9 g, 24 mmol) in DMF (30 mL) at 0° C. The reaction was allowed to slowly warm to ambient temperature over 1 h. After 2 h, the solution was diluted with brine, extracted with diethyl ether, washed twice with brine, dried over anhydrous Na₂SO₄, filtered, and concentrated to afford 12 g of crude 54 (the product contained unreacted acetanilide that co-eluted on TLC) which was used without further purification.

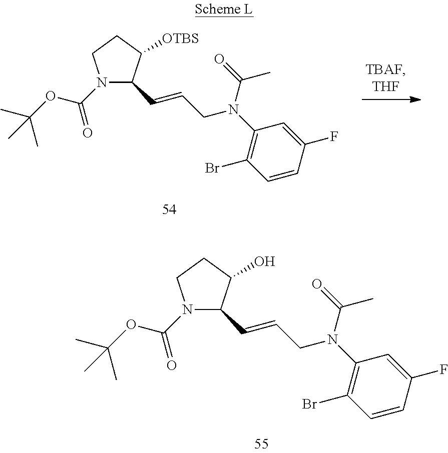

trans-2R-[2-{3-[Acetyl(2-bromo-5-fluorophenyl)amino]propenyl}]-3-hydroxypyrrolidine-1-carboxylic acid tert-butyl ester (55): To a solution containing crude 54 (11 g, approx., 19 mmol) in THF (30 mL) was added 1M TBAF/THF (25 mL) at ambient temperature. After 1 h, the solution was diluted with EtOAc, washed with 1M HCl, brine, dried over anhydrous Na₂SO₄, filtered, and concentrated. The residue was absorbed on silica gel and purified by flash silica gel chromatography (1:1 hexanes/EtOAc to 5% MeOH/DCM) to afford 4.2 g of alcohol 55 as an orange-colored foam. ¹H NMR (CDCl₃, 300 MHz) δ 7.65 (m, 1H), 7.04-7.02 (m, 2H), 5.62 (m, 1H), 5.40-5.34 (m, 1H), 4.74-4.69 (m, 1H), 4.26-4.00 (m, 2H), 3.74-3.38 (m, 3H), 2.69-2.57 (m, 1H), 1.82 (s, 3H), 1.46 (s, 9H) ppm.

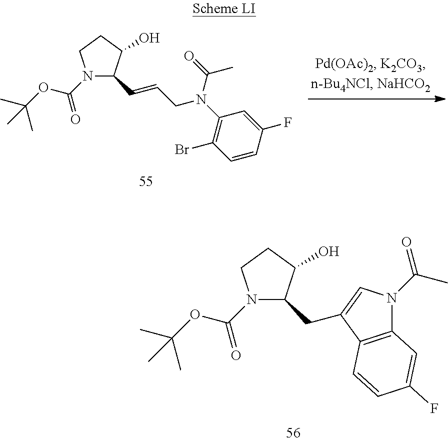

trans-2R-[2-(1-Acetyl-6-fluoro-1H-indol-3-ylmethyl)]-3-hydroxypyrrolidine-1-carboxylic acid tert-butyl ester (56): To a solution containing 55 (5.7 g, 12.5 mmol) in DMF (40 mL) was added K₂CO₃ (1.7 g, 12.3 mmol), sodium formate (0.86 g, 12.7 mmol), tetrabutylammonium chloride (3.5 g, 12.7 mmol), and Pd(OAc)₂ (0.32 g, 1.4 mmol) at ambient temperature. This reaction mixture was immersed in an oil bath preheated to 90° C. After 4 h, the reaction mixture was cooled in an ice bath, diluted with brine, extracted with EtOAc, washed twice with brine, dried over anhydrous Na₂SO₄, filtered, and concentrated to afford 4.5 g of crude indole 56 as an orange-colored foam that was used without further purification.

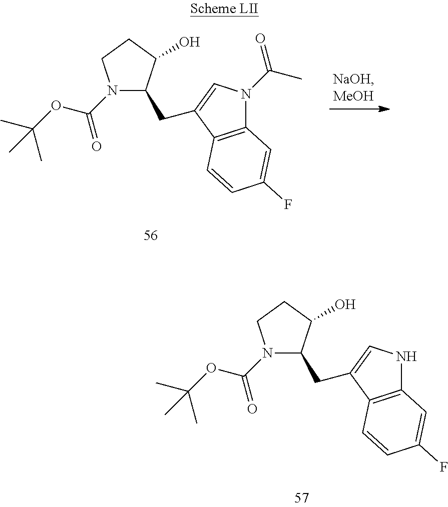

trans-2R-[2-(6-Fluoro-1H-indol-3-ylmethyl)]-3-hydroxypyrrolidine-1-carboxylic acid tert-butyl ester (57): To a solution containing acetate 56 (2.5 g, 6.6 mmol) in MeOH (15 mL) was added 1M NaOH (8 mL) at ambient temperature. After 40 min, the solution was concentrated, diluted with EtOAc, washed with brine, dried over anhydrous Na₂SO₄, filtered, and concentrated. The residue was purified by NP-HPLC (SiO₂, 40% EtOAc/hexanes increasing to EtOAc over 30 min) to afford 1.3 g of indole 57 as a light yellow-colored foam. ¹H NMR (CDCl₃, 300 MHz) δ 8.75 (s, rotomer, 0.5H), 8.71 (s, rotomer, 0.5H), 7.52 (dd, J=9.0, 14.1 Hz, 1H), 7.03-6.81 (m, 3H), 4.15-4.08 (m, 2H), 3.96 (dd, J=3.3, 10.2 Hz, 1H), 3.57-3.33 (m, 2H), 3.22-3.09 (m, 1H), 2.60-2.49 (m, 2H), 2.01-1.91 (m, 1H), 1.79-1.75 (m, 1H), 1.50 (s, 9H) ppm.

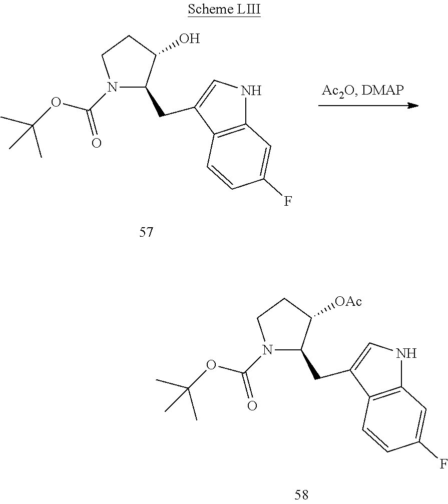

trans-2R-[3-Acetoxy-2-(6-fluoro-1H-indol-3-ylmethyl)]pyrrolidine-1-carboxylic acid tert-butyl ester (58): To a suspension containing indole 57 (0.35 g, 1.1 mmol) in DCM (10 mL) was added acetic anhydride (0.15 mL, 1.5 mmol) followed by DMAP (10 mg, 0.08 mmol) at ambient temperature. After 30 min, the solution became homogeneous. After 1 h, the solution was diluted with 1M HCl, extracted with DCM, dried over anhydrous Na₂SO₄, filtered, and concentrated to afford 0.36 g (87%) of 58 as a yellow-colored oil. ¹H NMR (CDCl₃, 300 MHz) δ 8.62 (s, rotomer, 0.5H), 8.57 (s, rotomer, 0.5H), 7.62-7.51 (m, 1H), 7.03 (d, J=7.8 Hz, 1H), 6.98 (s, 1H), 6.90-6.85 (m, 1H), 5.05 (s, 1H), 4.18-4.08 (m, 1H), 3.51-3.11 (m, 3H), 2.90-2.44 (m, 1H), 2.23 (s, 3H), 1.86-1.84 (m, 2H), 1.53 (s, 9H) ppm.

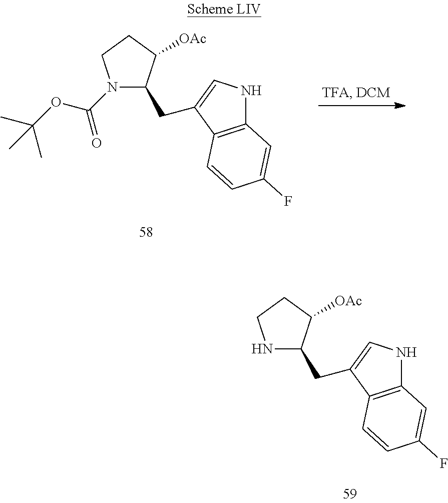

trans-2R-[Acetic acid 2-(6-fluoro-1H-indol-3-ylmethyl)]pyrrolidin-3-yl ester (59): To a solution containing carbamate 58 (0.48 g, 1.3 mmol) in DCM (15 mL) at 0° C. was added TFA (3 mL). After 15 min, the reaction was warmed and maintained at ambient temperature for 1 h. The solution was concentrated, diluted with EtOAc, washed with saturated NaHCO₃, dried over anhydrous Na₂SO₄, filtered, and concentrated to afford 0.32 g (89%) of amine 59 as an orange-colored oil that was used without further purification. ¹H NMR (CDCl₃, 300 MHz) δ 8.25 (s, 1H), 7.52 (dd, J=5.4, 8.7 Hz, 1H), 7.03-6.91 (m, 2H), 6.88 (ddd, J=0.9, 8.7, 17.4 Hz, 1H), 5.01-4.98 (m, 1H), 3.44 (m, 1H), 3.07-3.00 (m, 2H), 2.82 (dd, J=8.1, 14.7 Hz, 1H), 2.14-2.03 (m, 2H), 2.03 (s, 3H), 1.82-1.79 (m, 1H) ppm.

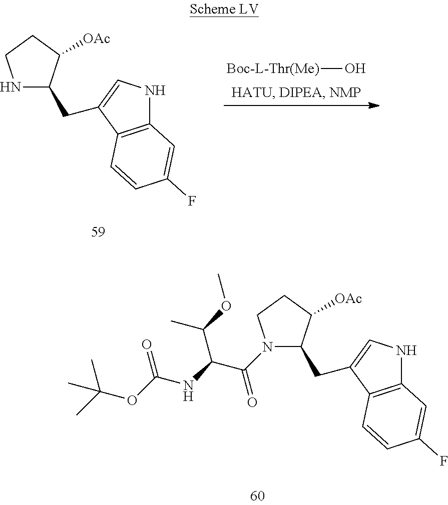

trans-2R-[Acetic acid 1-(2-tert-butoxycarbonylamino-3-methoxybutyryl)-2-(6-fluoro-1H-indol-3-ylmethyl)]pyrrolidin-3-yl ester (60): To a solution containing Boc-L-Thr(Me)-OH (105 mg, 0.45 mmol) in NMP (4 mL) at 0° C. was added HATU (169 mg, 0.44 mmol) followed by DIPEA (0.1 mL, 0.57 mmol). After 5 min, amine 59 (124 mg, 0.45 mmol) in NMP (5 mL) was added in a dropwise fashion. The reaction mixture was allowed to warm to ambient temperature. After 1 h, the solution was diluted with EtOAc, washed with 1M HCl, saturated aqueous NaHCO₃, brine, dried over anhydrous Na₂SO₄, filtered, and concentrated to afford 260 mg of amide 60 as an orange-colored oil that was used without further purification.

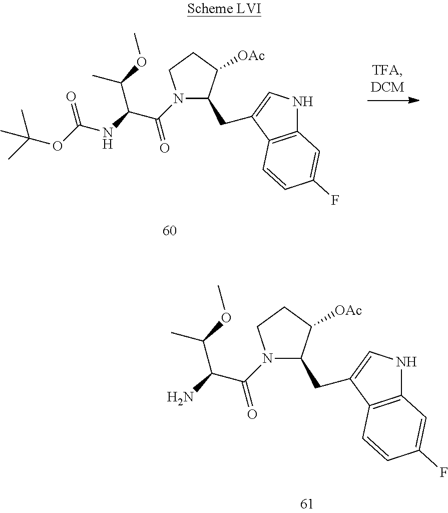

trans-2R-[Acetic acid 1-(2-amino-3-methoxybutyryl)-2-(6-fluoro-1H-indol-3-ylmethyl)]pyrrolidin-3-yl ester (61): To a solution containing 60 (0.26 g, 0.53 mmol) in DCM (15 mL) at 0° C. was added TFA (3 mL). After 15 min, the reaction mixture was warmed to ambient temperature. After 1 h, the solution was concentrated, diluted with EtOAc, washed twice with saturated aqueous NaHCO₃, dried over anhydrous Na₂SO₄, filtered, and concentrated to afford 0.20 g (97%) of amine 61 as an orange-colored oil that was used without further purification. ¹H NMR (CDCl₃, 300 MHz), mixture of amide rotamers: δ 8.75 (s, 0.3H), 8.31 (s, 0.7H), 7.80 (dd, J=5.4, 8.7 Hz, 0.7H), 7.45 (dd, J=5.4, 8.7 Hz, 0.3H), 7.07-7.00 (m, 2H), 6.94-6.87 (m, 1H), 5.17 (d, J=4.5 Hz, 0.3H), 5.07 (d, J=4.5 Hz, 0.7H), 4.53-4.43 (m, 1H), 3.80-3.69 (m, 2H), 3.43 (s, 2H), 3.26 (s, 1H), 3.58-3.18 (m, 1H), 2.94 (m, 1H), 2.54 (2m, 1H), 2.22-2.08 (m, 1H), 2.05 (s, 3H), 1.99 (s, 3H), 1.69 (m, 2H), 1.27 (d, J=6.9 Hz, 3H), 1.21 (d, J=6.9 Hz, 3H), 1.00 (d, J=6.3 Hz, 1H) ppm. Mass spectrum, m/z=[391.6] (M+).

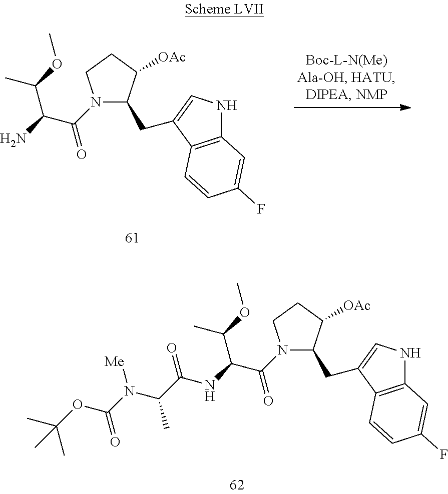

trans-2R-[Acetic acid 1-{2-[2-(tert-butoxycarbonylmethylamino)propionylamino]-3-methoxybutyryl}-2-(6-fluoro-1H-indol-3-ylmethyl)]pyrrolidin-3-yl ester (62): To a solution containing Boc-L-N(Me)-Ala-OH (47 mg, 0.23 mmol) in NMP (4 mL) at 0° C. was added HATU (88 mg, 0.23 mmol) followed by DIPEA (0.1 mL, 0.57 mmol). After 5 min, amine 61 (90 mg, 0.23 mmol) in NMP (5 mL) was added in a dropwise fashion. The reaction mixture was allowed to warm to ambient temperature. After 1 h, the solution was diluted with EtOAc, washed with 1M HCl, saturated aqueous NaHCO₃, brine, dried over anhydrous Na₂SO₄, filtered, and concentrated to afford 120 mg of amide 62 as an orange-colored oil that was used without further purification.

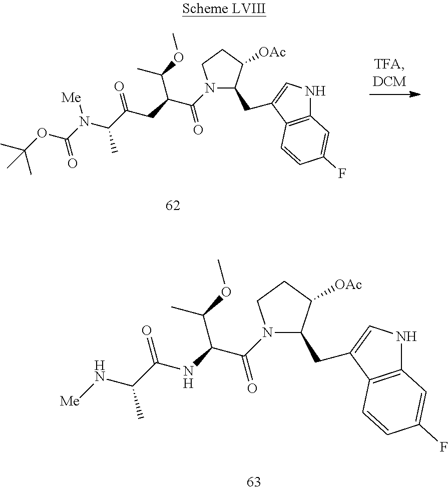

trans-2R-[Acetic acid 2-(6-fluoro-1H-indol-3-ylmethyl)-1-[3-methoxy-2-(2-methylaminopropionylamino)butyryl]]pyrrolidin-3-yl ester (63): To a solution containing carbamate 62 (120 mg, 0.21 mmol) in DCM (15 mL) at 0° C. was added TFA (3 mL). After 15 min, the reaction mixture was warmed to ambient temperature. After 1 h, the solution was concentrated, diluted with EtOAc, washed twice with saturated aqueous NaHCO₃, dried over anhydrous Na₂SO₄, filtered, and concentrated to afford 89 mg (89%) of amine 63 as a brown oil that was used without further purification. Mass spectrum, m/z=[476.5] (M)+.

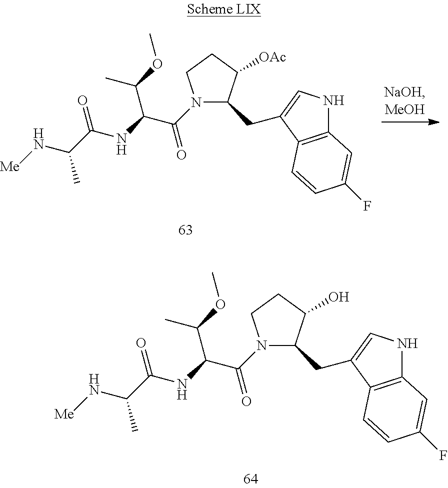

trans-2R—[N-{1-[2-(6-Fluoro-1H-indol-3-ylmethyl)-3-hydroxy-pyrrolidine-1-carbonyl]-2-methoxy-propyl}]-2-methylamino-propionamide (64): To a solution containing 63 (89 mg, 0.19 mmol) in MeOH (10 mL) was added 1M NaOH (1 mL) at ambient temperature. After 20 min, the solution was concentrated, diluted with water containing 0.1% HOAc and purified by RP-HPLC (Dynamax Microsorb C18 60 Å, 8μ, 41.4 mm×25 cm; Flow: 40 mL/min; Detector: 272 nm) using a 30 minute gradient method starting from 10% ACN/water w/0.1% v/v HOAc to 70% HOAc/water w/0.1% v/v HOAc. The product-containing fractions were frozen and lyophilized to afford 64 (44 mg) as an off-white solid. ¹H NMR (CDCl₃/d₄-MeOH, 300 MHz), mixture of amide rotamers, δ 8.65 (br s, 0.3H), 8.45 (br s, 0.7H), 8.12 (br s, 1H), 7.68-7.64 (m, 1H), 7.53 (app d, J=8.4 Hz, 0.3H), 7.38 (app q, J=5.4 Hz, 0.7H), 7.09-6.98 (m, 2H), 6.90-6.84 (m, 1H), 4.86 (br s, 1H), 4.54-4.41 (m, 1H), 4.30 (app d, J=3.9 Hz, 0.3H), 4.22 (br s, 0.7H), 3.95-3.79 (m, 2H), 3.69-3.63 (m, 1H), 3.50 (m, 0.5H), 3.26 (m, 0.5H), 3.41 (s, 2H), 3.33 (s, 1H), 2.93 (app q, J=6.9 Hz, 0.5H), 2.82 (app d, J=7.2 Hz, 0.5H), 2.48 (app q, J=10.8 Hz, 1H), 2.34 (s, 2H), 2.26 (s, 1H), 1.28 (app d, J=6.9 Hz, 1.5H), 1.21 (app d, J=6.3 Hz, 1.5H), 1.02 (d, J=6.3 Hz, 1H) ppm. Mass spectrum, m/z=[434.5] (M)+.

Using the general procedures outlined in Schemes XLIX through LIX and the appropriate amino acid analogues to the amino acid reagents Boc-Thr(Me)-OH and Boc-N(Me)Ala-OH, the compounds reported in Table 8 were prepared and tested for their binding affinities (Kd) to XIAP BIR-3 or cIAP-1 BIR-3.

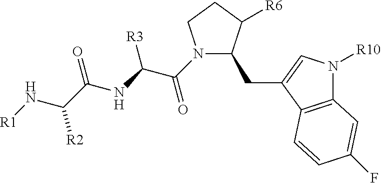

TABLE 8

| | | | | | | | Observed |
|---|---|---|---|---|---|---|---|
| Com- | | | | | | Kd | Mass |
| pound | R1 | R2 | R3 | R6 | R10 | (μM) | (m/z) |
| RR | Me | Me | (2R—EtOMe) | (S)—OH | H | A | 434.5 |
| SS | Et | Me | (2R—EtOMe) | (S)—OH | H | A | 448.6 |
| TT | Me | Et | (2R—EtOMe) | (S)—OH | H | A | 448.6 |
| UU | Me | Me | tert-Butyl | (S)—OH | H | A | 432.6 |
| VV | Me | Et | tert-Butyl | (S)—OH | H | A | 446.5 |
| WW | Me | Me | cyclo-Hexyl | (S)—OH | H | A | 458.6 |
| XX | Me | Et | cyclo-Hexyl | (S)—OH | H | A | 472.5 |
| YY | Me | Me | (2R—EtOMe) | (S)—OH | Me | A | 448.6 |
| ZZ | Et | Me | (2R—EtOMe) | (S)—OH | Me | A | 462.6 |
| A′ | Et | Me | (2R—EtOMe) | (S)—OMe | Me | B | 476.7 |
| B′ | Me | Me | (2R—EtOMe) | (S)—OMe | Me | A | 462.6 |
| C′ | Me | Et | (2R—EtOMe) | (S)—OMe | Me | D | 476.6 |
| D′ | Me | Et | (2R—EtOMe) | (S)—OMe | H | D | 462.7 |
| E′ | Me | Me | tert-Butyl | (S)—OMe | Me | D | 460.6 |
| F′ | Me | Et | tert-Butyl | (S)—OMe | Me | D | 474.6 |
| G′ | Et | Me | (2R—EtOMe) | (S)—OMe | H | D | 462.7 |
| H′ | Me | Me | tert-Butyl | (S)—OMe | H | D | 446.7 |
| I′ | Et | Me | tert-Butyl | (S)—OMe | H | D | 460.7 |
| J′ | Me | Et | tert-Butyl | (S)—OMe | H | D | 460.7 |
| K′ | Et | Me | tert-Butyl | (S)—OMe | Me | D | 474.7 |
| L′ | Me | Me | (2R—EtOMe) | (S)—OMe | H | D | 448.5 |
| M′ | Me | Me | (2R—EtOMe) | (R)—OH | H | A | 434.6 |

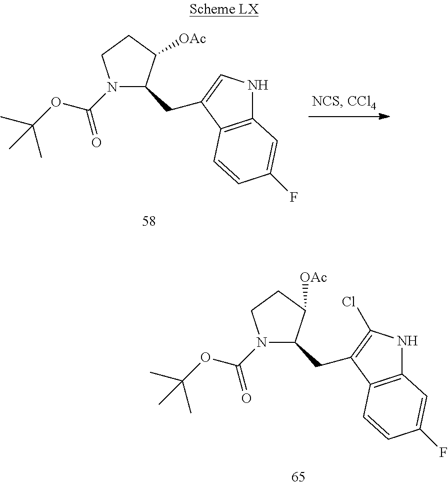

3-Acetoxy-2-(2-chloro-6-fluoro-1H-indol-3-ylmethyl)-pyrrolidine-1-carboxylic acid tert-butyl ester (65): A solution containing 58 (See Scheme LIII) (1.8 g, 4.8 mmol) in CCl₄ (30 mL) was treated with benzoyl peroxide (21 mg, 0.09 mmol) followed by NCS (649 mg, 4.9 mmol) at ambient temperature. The reaction mixture was warmed to reflux. After 1 h, the reaction mixture was concentrated onto silica gel and chromatographed (3:1 hexanes/EtOAc) to afford 1.2 g (63%) of 65 as an amber-colored foam.

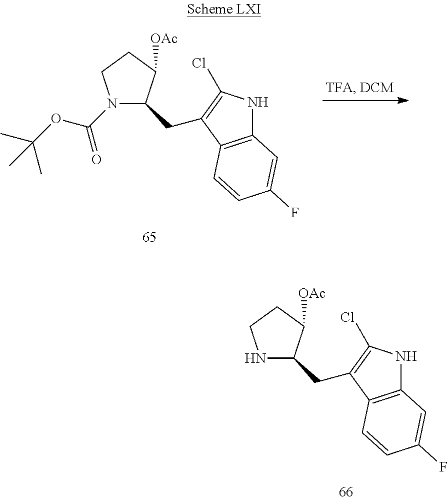

Acetic acid 2-(2-chloro-6-fluoro-1H-indol-3-ylmethyl)-pyrrolidin-3-yl ester (66): A solution containing 65 (1.2 g, 2.92 mmol) in DCM (20 mL) at 0° C. was treated with TFA (5 mL). After 4 h, the reaction mixture was concentrated in vacuo and the residue was dissolved in EtOAc, washed successively with aqueous NaHCO₃ (2×), brine, dried over anhydrous Na₂SO₄, filtered, and concentrated to afford 0.9 g (99%) of 66 which was used without further purification. Mass spectrum, m/z=[310.9] (M)+.

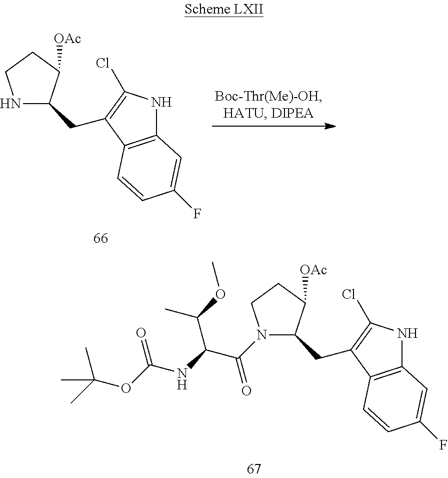

Acetic acid 1-(2-tert-butoxycarbonylamino-3-methoxy-butyryl)-2-(2-chloro-6-fluoro-1H-indol-3-ylmethyl)-pyrrolidin-3-yl ester (67): To a solution containing amine 66 (225 mg, 0.72 mmol), Boc-Thr(Me)-OH (177 mg, 0.75 mmol), and HATU (289 mg, 0.76 mmol) in NMP (4 mL) at 0° C. was added DIPEA (110 mg, 0.86 mmol). The reaction mixture was allowed to warm to ambient temperature. After 2 h, reaction mixture was diluted with diethyl ether and washed successively with dilute aqueous HCl, water (5×), aqueous NaHCO₃, water (2×), then brine. The organic phase was dried with anhydrous Na₂SO₄, filtered, and concentrated to afford the crude product which was purified by flash silica gel chromatography (1:1 hexanes/EtOAc) to afford 146 mg (38%) of 67 as a tan-colored foam. Mass spectrum, m/z=[526.0] (M)+.

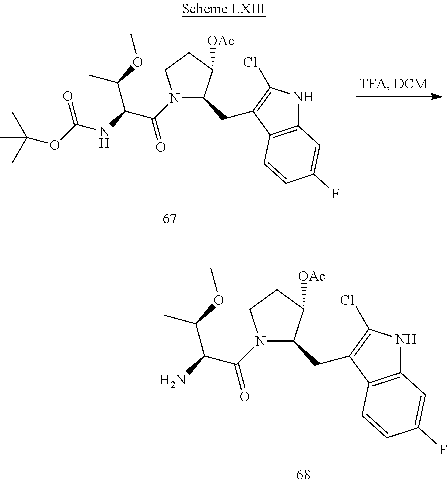

Acetic acid 1-(2-amino-3-methoxy-butyryl)-2-(2-chloro-6-fluoro-1H-indol-3-ylmethyl)-pyrrolidin-3-yl ester (68): A solution containing 67 (145 mg, 0.27 mmol) in DCM (10 mL) at 0° C. was treated with TFA (2 mL). After 40 min, the reaction mixture was concentrated in vacuo and the residue was dissolved in EtOAc, washed successively with aqueous NaHCO₃ (2×), brine, dried over anhydrous Na₂SO₄, filtered, and concentrated to afford 101 mg (86%) of 68 which was used without further purification. Mass spectrum, m/z=[425.9] (M)+.

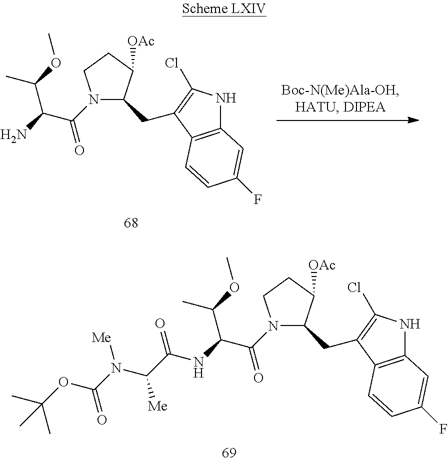

Acetic acid 1-{2-[2-(tert-butoxycarbonyl-methyl-amino)-propionylamino]-3-methoxy-butyryl}-2-(2-chloro-6-fluoro-1H-indol-3-ylmethyl)-pyrrolidin-3-yl ester (69): To a solution containing amine 68 (50 mg, 0.12 mmol), Boc-N(Me)Ala-OH (25 mg, 0.12 mmol), and HATU (47 mg, 0.12 mmol) in NMP (3 mL) at 0° C. was added DIPEA (15 mg, 0.12 mmol). The reaction mixture was allowed to warm to ambient temperature. After 2 h, reaction mixture was diluted with diethyl ether and washed successively with dilute aqueous HCl, water (5×), aqueous NaHCO₃, water (2×), then brine. The organic phase was dried with anhydrous Na₂SO₄, filtered, and concentrated to afford the crude product which was purified by flash silica gel chromatography (1:1 hexanes/EtOAc) to afford 72 mg (99%) of 69 which was used without further purification. Mass spectrum, m/z=[611.1] (M)+.

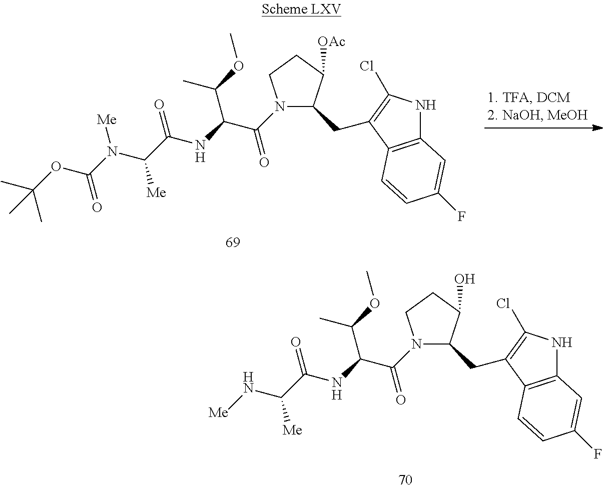

N-{1-[2-(2-Chloro-6-fluoro-1H-indol-3-ylmethyl)-3-hydroxy-pyrrolidine-1-carbonyl]-2-methoxy-propyl}-2-methylamino-propionamide (70): A solution containing 69 (72 mg, 0.12 mmol) in DCM (10 mL) at 0° C. was treated with TFA (2 mL). After 1 h, the reaction mixture was concentrated in vacuo and the residue was dissolved in EtOAc, washed successively with aqueous NaHCO₃ (2×), brine, dried over anhydrous Na₂SO₄, filtered, and concentrated. Mass spectrum, m/z=[511] (M)+.

The residue was dissolved in MeOH (5 mL) and cooled to 0° C. Aqueous NaOH (1M, 0.14 mL) was added. After 30 min, the reaction mixture was warmed to ambient temperature. After 30 min, the solvent was removed in vacuo and the residue was purified by reverse-phase HPLC (2″ Dynamax C18 column; A: water w/0.1% v/v HOAc; B: ACN w/0.1% v/v HOAc; Method: 10-70% B over 30 min; Flow: 40 mL/min) to afford 16 mg of 70 as a white solid following lyophilization. Mass spectrum, m/z=[468.9] (M)+.

Using the general procedures outlined in Schemes LX through LXV and the appropriate amino acid analogues to the amino acid reagents Boc-Thr(Me)-OH and Boc-N(Me)Ala-OH, the compounds reported in Table 9 were prepared and tested for their binding affinities (Kd) to XIAP BIR-3 or cIAP-1 BIR-3.

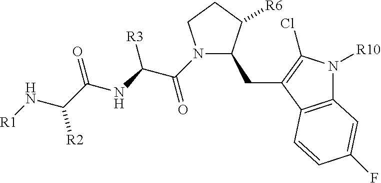

TABLE 9

| | | | | | | | Observed Mass |
|---|---|---|---|---|---|---|---|
| Compound | R1 | R2 | R3 | R6 | R10 | Kd (μM) | (m/z) |
| N′ | Me | Me | (2R—EtOH) | (S)—OMe | H | B | 469.1 |
| O′ | Me | Et | (2R—EtOH) | (S)—OMe | H | B | 483.1 |
| P′ | Me | Me | 2R—EtOMe) | (S)—OMe | H | A | 483 |
| Q′ | Me | Et | (2R—EtOMe) | (S)—OMe | H | B | 497 |
| R′ | Et | Me | (2R—EtOMe) | (S)—OMe | H | B | 497.1 |
| S′ | Me | CH₂OH | (2R—EtOMe) | (S)—OMe | H | C | 499.1 |
| T′ | Me | Me | i-Pr | (S)—OMe | H | B (cIAP-1) | 467.1 |
| U′ | Et | Me | iPr | (S)—OMe | H | B (cIAP-1) | 481.1 |
| V′ | Me | Et | iPr | (S)—OMe | H | B (cIAP-1) | 481.1 |
| W′ | Me | Me | Cyclohexyl | (S)—OH | H | A (cIAP-1) | 492.9 |
| X′ | Me | Et | tert-Butyl | (S)—OH | H | A (cIAP-1) | 481 |
| Y′ | Me | Me | tert-Butyl | (S)—OH | H | A (cIAP-1) | 467 |
| Z′ | Me | Et | iPr | (S)—OH | H | A (cIAP-1) | 467 |
| AA′ | Me | Me | iPr | (S)—OH | H | A (cIAP-1) | 495 |
| BB′ | Me | Et | 2R—EtOMe | (S)—OH | H | A (cIAP-1) | 483 |
| CC′ | Me | Me | 2R—EtOMe | (S)—OH | H | A (cIAP-1) | 468 |

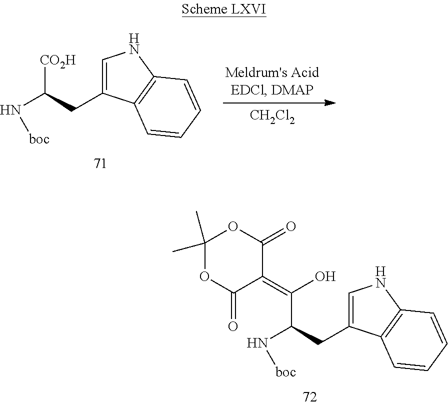

[2-(2,2-Dimethyl-4,6-dioxo-[1,3]dioxan-5-ylidene)-2-hydroxy-1-(1H-indol-3-ylmethyl)-ethyl]-carbamic acid tert-butyl ester (72): To a well-stirred suspension of Boc-D-Trp-OH (71, 12.5 g, 41.0 mmol) and Meldrum's acid (5.92 g, 41.0 mmol) in CH₂Cl₂ (205 mL) at 0° C. were added DMAP (11.8 g, 61.6 mmol) and EDCI (7.55 g, 61.6 mmol) at which time the reaction became a pale yellow homogeneous solution. The reaction mixture was allowed to slowly warm to ambient temperature. After 16 h, the reaction mixture was diluted with CH₂Cl₂ and washed with 10% KHSO₄, and brine. The organic phase was dried over anhydrous Na₂SO₄, filtered and concentrated to afford 72 (17.1 g, 96%) as an off-white solid which was used without further purification. ¹H NMR (CDCl₃, 300 MHz) δ 8.19 (br s, 1H), 7.71 (m, 1H), 7.34 (d, J=7.5 Hz, 1H), 7.21-7.06 (m, 4H), 5.96 (d, J=5.7 Hz, 1H), 5.12 (m, 1H), 3.35 (m, 1H), 3.13 (m, 1H), 1.73 (s, 3H), 1.58 (s, 3H), 1.35 (m, 9H) ppm.

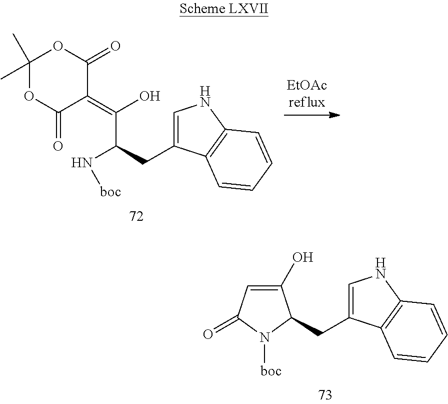

3-Hydroxy-2-(1H-indol-3-ylmethyl)-5-oxo-2,5-dihydro-pyrrole-1-carboxylic acid tert-butyl ester (73): A well-stirred solution of 72 (17.1 g, 39.6 mmol) in EtOAc (300 mL) was heated to reflux in a preheated oil bath. After 1 h, the reaction mixture was cooled to ambient temperature. The EtOAc solution was extracted 5×100 mL NaHCO₃ (sat.), and the combined aqueous extracts were acidified to pH=2 using 3 M HCl. The resultant aqueous phase was extracted 4×EtOAc and the combined extracts were washed with brine, dried over anhydrous Na₂SO₄, filtered and concentrated to afford 73 (13.5 g, >100%) as a foamy white solid which was taken forward without further purification. ¹H NMR (CDCl₃, 300 MHz) δ 8.32 & 8.17 (br s, 1H, rotamers), 7.62-6.92 (m, 5H), 4.63 (s, 1H), 3.50 (t, J=3.9 Hz), 2.79 (d, J=22.5 Hz, 1H), 2.21 (d, J=23.1 Hz), 1.63 (br s, 9H) ppm. Mass spectrum, m/z=[328.1] (M)+.

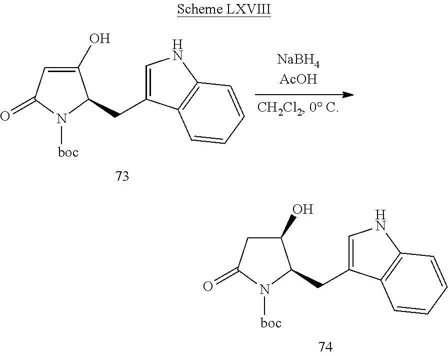

3-Hydroxy-2-(1H-indol-3-ylmethyl)-5-oxo-pyrrolidine-1-carboxylic acid tert-butyl ester (74): To a well-stirred solution of 73 (13.0 g, 39.6 mmol) in CH₂Cl₂ (200 mL) and AcOH (25 mL) at 0° C. was added NaBH₄ (3.27 g, 83.2 mmol) portion-wise. The reaction mixture was continued stirring at 0° C. for 2.5 h, after which time the reaction mixture was quenched with H₂O. The layers were separated and the aqueous phase was extracted with CH₂Cl₂. The combined organic extracts were washed successively with 3×H₂O and brine, dried over anhydrous Na₂SO₄, filtered and concentrated to afford an off-white solid. This crude material was purified through a plug of SiO₂ (eluting with 1:1 EtOAc/hexanes) to afford 74 (11.9 g, 91%) as a foamy white solid. ¹H NMR (CDCl₃, 300 MHz) δ 8.49 (br s, 1H), 7.70 (d, J=7.5 Hz, 1H), 7.36 (d, J=7.8 Hz, 1H), 7.15 (dt, J=6.6, 18 Hz, 2H), 7.01 (d, J=1.8 Hz, 1H), 4.50 (q, J=6.3, 12.3 Hz, 1H), 4.37 (q, J=7.5, 14.7 Hz, 1H), 3.28 (m, 2H), 2.52 (dd, J=7.8, 17.4 Hz, 1H), 2.26 (dd, J=7.8, 17.4 Hz, 1H), 1.44 (s, 9H) ppm. Mass spectrum, m/z=[330.2] (M)+.

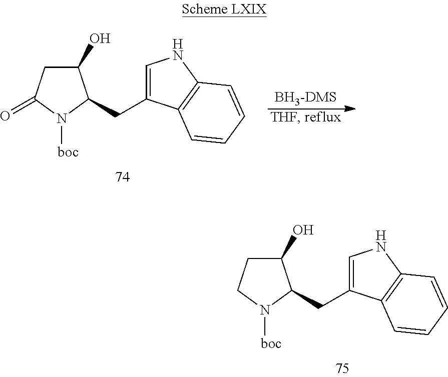

3-Hydroxy-2-(1H-indol-3-ylmethyl)-pyrrolidine-1-carboxylic acid tert-butyl ester (75): To a well-stirred solution of 74 (11.9 g, 36.0 mmol) in THF (180 mL) at ambient temperature was added a 2.0 M/THF solution of BH₃.DMS (54 mL, 108.1 mmol) dropwise over 30 min during which time gas evolution was observed. The resultant yellow solution was heated to reflux in a preheated oil bath. After 4 h, the pale green reaction mixture was cooled to ambient temperature, poured into Et₂O (600 mL) and quenched with NH₄Cl (sat.). The layers were separated and the organic phase was washed successively with 5% citric acid, H₂O and brine. The resultant organic layer was dried over anhydrous Na₂SO₄, filtered and concentrated to afford 75 (8.09 g, 71%) as a foamy white solid which was used without further purification. ¹H NMR (CDCl₃, 300 MHz) δ 8.17 (br s, 1H), 7.75 (br s, 1H), 7.36 (d, J=8.1 Hz, 1H), 7.17 (dt, J=0.9, 6.9 Hz, 1H), 7.12 (dt, J=1.2, 8.1 Hz), 7.08 (s, 1H), 4.22 (m, 2H), 3.44 (m, 3H), 3.09 (dd, J=9, 14.4 Hz, 1H), 1.90 (s, 1H), 1.72 (m, 1H), 1.46 (s, 9H) ppm. Mass spectrum, m/z=[316.8] (M)+.

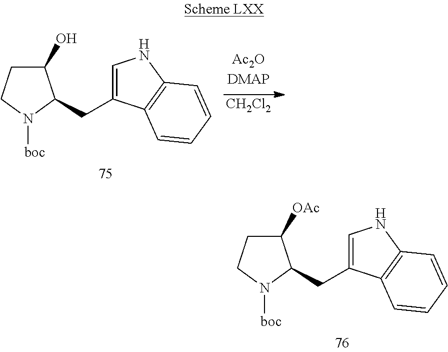

3-Acetoxy-2-(1H-indol-3-ylmethyl)-pyrrolidine-1-carboxylic acid tert-butyl ester (76): To a well-stirred suspension of 75 (8.09 g, 25.5 mmol) in CH₂Cl₂ (125 mL) was added DMAP (cat.) and Ac₂O (3.63 mL, 38.3 mmol) at which time the reaction became yellow and homogeneous. The reaction mixture was continued stirring for 18 h, during which time the color changed from yellow to red. The reaction mixture was diluted with CH₂Cl₂ and washed successively with 1 M HCl, NaHCO₃ (sat.) and brine. The resultant organic layer was dried over anhydrous Na₂SO₄, filtered and concentrated. The foamy brown solid was adsorbed onto SiO₂ and purified via flash chromatography (SiO₂, 2:1 hexanes/EtOAc) to afford 76 (4.73 g, 52%) as a foamy white solid. ¹H NMR (CDCl₃, 300 MHz) δ 8.19 (bs, 1H), 7.67 (bs, 1H), 7.33 (d, 7.8 Hz, 1H), 7.17 (dt, J=0.9, 7.2 Hz, 1H), 7.10 (dt, J=1.2, 7.8 Hz, 1H), 6.93 (s, 1H), 5.14 (q, J=6.0 Hz, 1H), 4.39 (q, J=6.0 Hz, 1H), 3.50 (m, 1H), 3.37 (m, 1H), 2.10-1.80 (m, 5H), 1.39 (m, 9H) ppm. Mass spectrum, m/z=[358.8] (M)+.

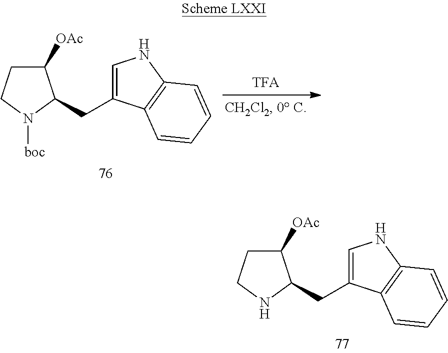

Acetic acid 2-(1H-indol-3-ylmethyl)-pyrrolidin-3-yl ester (77): To a well-stirred solution of 76 (2.61 g, 7.28 mmol) in CH₂Cl₂ (35 mL) at 0° C. was added TFA (8 mL). The resultant dark green solution was stirred for an additional 2 h after which time the reaction was concentrated. The residue was taken up in CH₂Cl₂ and washed 2×NaHCO₃ (sat.) and brine. The resultant organic phase was dried over anhydrous Na₂SO₄, filtered and concentrated to afford 77 (1.78 g, 95%) as a foamy pale yellow solid which was used without further purification. ¹H NMR (CDCl₃, 300 MHz) δ 7.28 (bs, 1H), 7.59 (d, J=7.8 Hz, 1H), 7.34 (d, J=7.5 Hz, 1H), 7.18 (t, J=6.9 Hz, 1H), 7.10 (t, J=7.5 Hz, 1H), 7.04 (s, 1H), 5.22 (m, 1H), 3.42 (m, 1H), 3.20 (m, 2H), 3.03 (m, 2H), 2.87 (m, 1H), 2.13 (s, 3H), 1.91 (m, 2H) ppm. Mass spectrum, m/z=[258.8] (M)+.

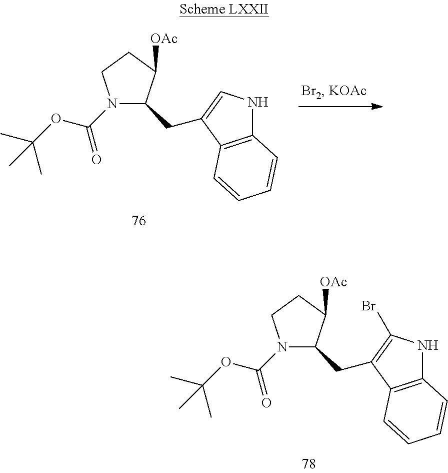

3-Acetoxy-2-(2-bromo-1H-indol-3-ylmethyl)-pyrrolidine-1-carboxylic acid tert-butyl ester (78): To a solution containing 76 (7.62 g, 21.3 mmol) in CHCl₃ (215 mL) at 0° C. was added KOAc (6.26 g, 63.7 mmol) followed by the dropwise addition of Br, (4.07 g, 25.4 mmol) in CHCl₃ (8 mL). After 15 min, the heterogeneous reaction mixture was diluted with brine and DCM. The layers were separated and the organic phase was washed successively with 10% aqueous Na₂S₂O₃ and brine, dried over anhydrous Na₂SO₄, filtered, and concentrated. The crude bromide was purified by flash silica gel chromatography (2:1 hexanes/EtOAc to 1:3 hexanes/EtOAc) to afford 6.31 g (68%) of 78. Mass spectrum, m/z=[436.8] (M)+.

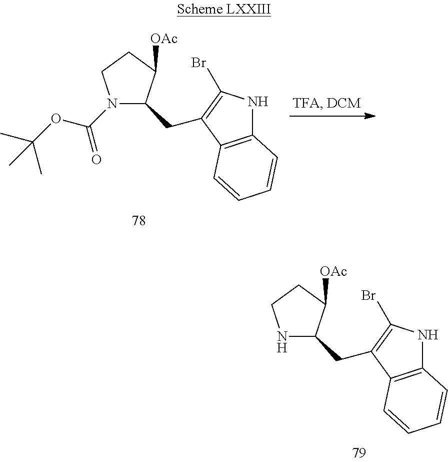

Acetic acid 2-(2-bromo-1H-indol-3-ylmethyl)-pyrrolidin-3-yl ester (79): A solution containing 78 (3.24 g, 7.40 mmol) in DCM (20 mL) was treated with TFA (4 mL) at 0° C. Additional TFA was added as needed over 7 h. Upon complete consumption of 78, the reaction mixture was concentrated in vacuo. The crude product was purified by reverse-phase HPLC (2″ Dynamax C18 column; A: water w/0.1% v/v HOAc; B: ACN w/0.1% v/v HOAc; Method: 10-100% B over 30 min; Flow: 40 mL/min). The product-containing fractions were combined and concentrated in vacuo to remove ACN. The aqueous solution was partitioned with EtOAc and washed successively with aqueous NaHCO₃ and brine. The aqueous washes were back extracted with EtOAc and the combined organic extracts were dried over anhydrous Na₂SO₄, filtered, and concentrated to afford 1.09 g (44%) of 79.

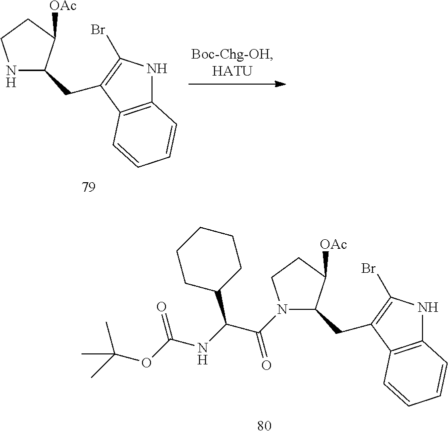

Acetic acid 2-(2-bromo-1H-indol-3-ylmethyl)-1-(2-tert-butoxycarbonylamino-2-cyclohexyl-acetyl)-pyrrolidin-3-yl ester (80): To a solution containing amine 79 (0.34 g, 1.00 mmol), Boc-Chg-OH (285 mg, 1.11 mmol), and HATU (460 mg, 1.21 mmol) in NMP (5 mL) at 0° C. was added DIPEA (169 mg, 1.31 mmol). The reaction mixture was allowed to warm to ambient temperature over night. The reaction mixture was diluted with diethyl ether and washed successively with dilute aqueous HCl, water (5×), aqueous NaHCO₃, water (2×), then brine. The aqueous washes were back extracted with diethyl ether and the combined organic extracts were dried with anhydrous Na₂SO₄, filtered, and concentrated to afford 0.66 g (>100%) of crude 80 which was used without further purification.

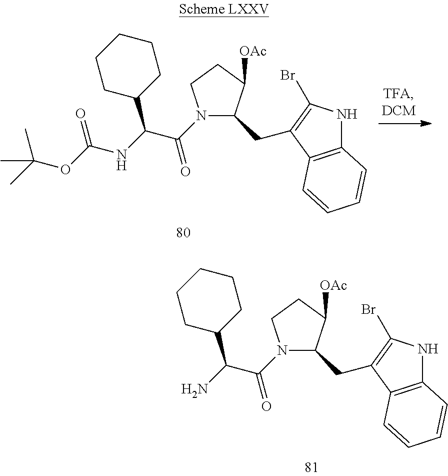

Acetic acid 1-(2-amino-2-cyclohexyl-acetyl)-2-(2-bromo-1H-indol-3-ylmethyl)-pyrrolidin-3-yl ester (81): A solution containing crude 80 (0.66 g) in DCM (10 mL) was treated with TFA (2 mL) at 0° C. After 1 h, the reaction mixture was concentrated in vacuo. The crude residue was diluted with EtOAc and washed successively with aqueous NaHCO₃ (2×) and brine. The combined aqueous washes were back extracted with EtOAc and the combined organic extracts were dried over anhydrous Na₂SO₄, filtered, and concentrated to afford 0.16 g (33%, 2 steps) of 81 which was used directly without further purification.

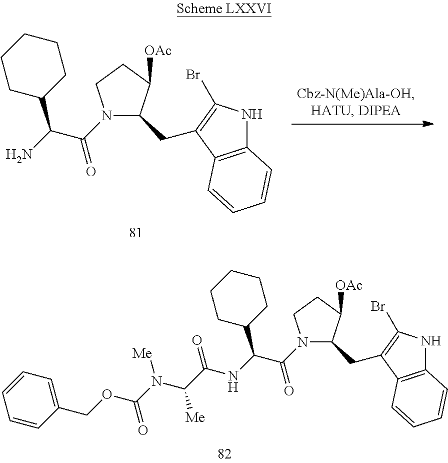

Acetic acid 1-{2-[2-(benzyloxycarbonyl-methyl-amino)-propionylamino]-2-cyclohexyl-acetyl}-2-(2-bromo-1H-indol-3-ylmethyl)-pyrrolidin-3-yl ester (82): To a solution containing crude amine 81 (0.16 g, 0.33 mmol), Cbz-N(Me)Ala-OH (87 mg, 0.36 mmol), and HATU (153 mg, 0.40 mmol) in NMP (5 mL) at 0° C. was added DIPEA (56 mg, 0.43 mmol). The reaction mixture was allowed to warm to ambient temperature over night. The reaction mixture was diluted with diethyl ether and washed successively with dilute aqueous HCl, water (5×), aqueous NaHCO₃, water (2×), then brine. The aqueous washes were back extracted with diethyl ether and the combined organic extracts were dried with anhydrous Na₂SO₄, filtered, and concentrated to afford 0.27 g (>100%) of crude 82 which was used without further purification. Mass spectrum, m/z=[697.0] (M+H)+.

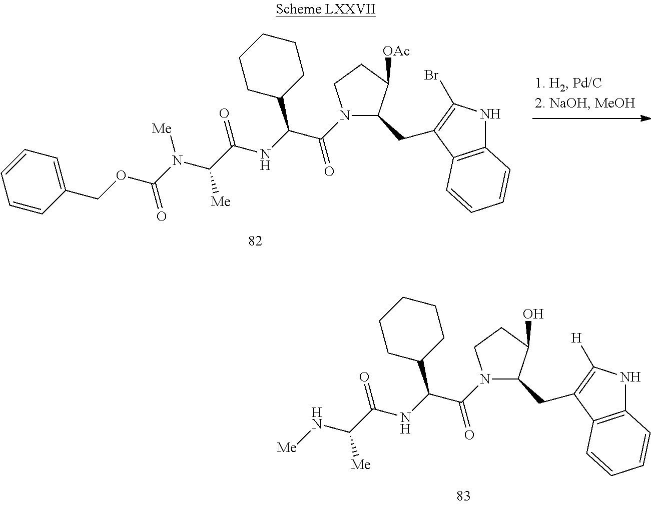

N-{1-Cyclohexyl-2-[3-hydroxy-2-(1H-indol-3-ylmethyl)-pyrrolidin-1-yl]-2-oxo-ethyl}-2-methylamino-propionamide (83): A mixture containing crude 82 (0.27 g) and 10% Pd-on-C (˜0.1 g) in MeOH (20 mL) was placed in a Parr bottle and pressurized to 50-55 PSI (3.4-3.7 atm) hydrogen. After 2 hr of shaking on a Parr apparatus, the reaction mixture was filtered and the solids were washed with MeOH. The filtrate was concentrated in vacuo and the residue was dissolved in MeOH (10 mL). At 0° C., aqueous NaOH (1M, 2 mL) was added. After 2 h, glacial HOAc (4 mL) was added and the reaction mixture was concentrated in vacuo. The residue was dissolved in water/ACN containing 0.1% v/v HOAc and the product was purified by reverse-phase HPLC (2″ Dynamax C18 column; A: water w/0.1% v/v HOAc; B: ACN w/0.1% v/v HOAc; Method: 10-70% B over 30 min; Flow: 40 mL/min) to afford 67.4 mg (39%, 2 steps) of the acid addition salt 83.HOAc as a white solid following lyophilization. Mass spectrum, m/z=[441.0] (M)+.

Using the general procedures outlined in Schemes LXVI through LXXVII and the appropriate amino acid analogues to the amino acid reagents Boc-Chg-OH and Cbz-N(Me)Ala-OH, the compounds reported in Table 10 were prepared and tested for their binding affinities (Kd) to XIAP BIR-3 or cIAP-1 BIR-3.

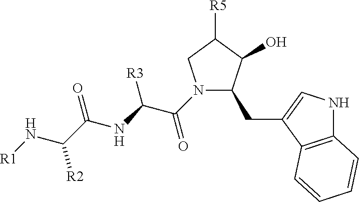

TABLE 10

| | | | | | | Observed |
|---|---|---|---|---|---|---|
| | | | | | | Mass |
| Compound | R1 | R2 | R3 | R5 | Kd (μM) | (m/z) |
| DD′ | Et | Me | 2R—EtOH | H | A (cIAP-1) | 417.0 |
| EE′ | Me | Et | 2R—EtOH | H | A | 416.9 |
| FF′ | Me | Me | 2R—EtOH | H | A | 402.9 |
| GG′ | Et | Me | 2R—EtOMe | H | A (cIAP-1) | 431.0 |
| HH′ | Me | Et | 2R—EtOMe | H | A | 431.0 |
| II′ | Me | Me | 2R—EtOMe | H | A | 417.0 |
| JJ′ | Et | Me | Cyclohexyl | H | A (cIAP-1) | 455.0 |
| KK′ | Me | Et | Cyclohexyl | H | A (cIAP-1) | 455.0 |
| LL′ | Me | Me | Cyclohexyl | H | A | 441.0 |
| MM′ | Me | cPr | tert-Butyl | H | A (cIAP-1) | 441 |
| NN′ | Me | Et | tert-Butyl | H | A (cIAP-1) | 429 |
| OO′ | Et | Me | tert-Butyl | H | A (cIAP-1) | 429 |
| PP′ | Me | Me | tert-Butyl | H | A (cIAP-1) | 415 |
| QQ′ | Me | cPr | Cyclopropyl | H | B (cIAP-1) | 424.9 |
| RR′ | Me | Et | Cyclopropyl | H | A (cIAP-1) | 413 |
| SS′ | Et | Me | Cyclopropyl | H | B (cIAP-1) | 412.9 |
| TT′ | Me | cPr | iPr | H | B (cIAP-1) | 427 |
| UU′ | Me | Et | iPr | H | A (cIAP-1) | 415 |
| VV′ | Me | Me | Cyclopropyl | H | A (cIAP-1) | 398.9 |
| WW′ | Et | Me | iPr | H | A (cIAP-1) | 415.0 |
| XX′ | Me | Me | iPr | H | A (cIAP-1) | 401 |

Using the general procedures outlined in Schemes LXVI through LXXVII and the appropriate amino acid analogues to the amino acid reagents Boc-Chg-OH and Cbz-N(Me)Ala-OH, the compound reported in Table 11 were prepared and tested for its binding affinities (Kd) to XIAP BIR-3 or cIAP-1 BIR-3.

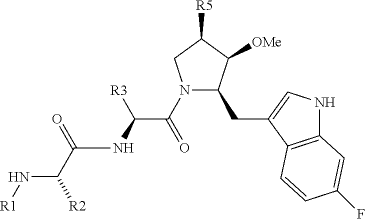

TABLE 11

| | | | | | | Observed Mass |
|---|---|---|---|---|---|---|
| Compound | R1 | R2 | R3 | R5 | Kd (μM) | (m/z) |
| YY′ | Me | Me | 2R—EtOMe | H | A | 449 |

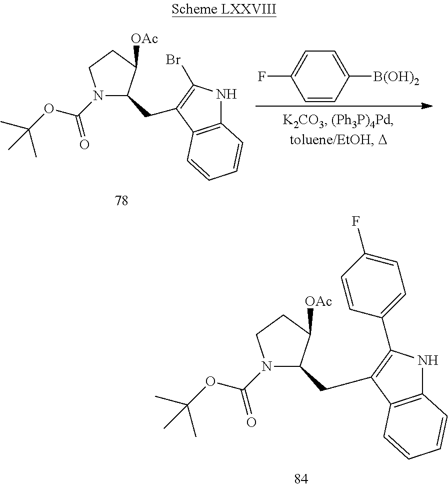

2-[2-(4-Fluoro-phenyl)-1H-indol-3-ylmethyl]-3-hydroxy-pyrrolidine-1-carboxylic acid tert-butyl ester (84): A mixture containing 78 (See Scheme LXXII)(1.1 g, 2.52 mmol), K₂CO₃ (1.22 g, 8.82 mmol), 4-F-phenylboronic acid (458 mg, 3.27 mmol), and (Ph₃P)₄Pd (145 mg, 5 mol %) was heated at 85° C. for 5 h. The reaction mixture was cooled to ambient temperature and diluted with EtOAc. The organic solution was washed successively with 1N HCl and brine, dried over anhydrous Na₂SO₄, filtered, and concentrated. The crude product was purified by silica gel chromatography to afford 920 mg (81%) of 84 as a yellow-colored solid. Mass spectrum, m/z=[452.9] (M)+.

Using the general procedures outlined in Schemes LXXIII through LXXVII and the appropriate amino acid analogues to the amino acid reagents Boc-Chg-OH and Cbz-N(Me)Ala-OH, the compounds reported in Table 12 were prepared and tested for their binding affinities (Kd) to XIAP BIR-3 or cIAP-1 BIR-3.

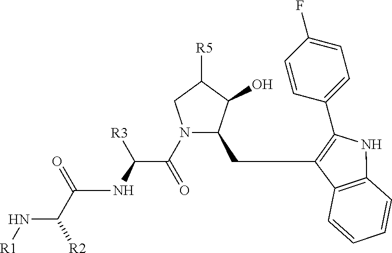

TABLE 12

| | | | | | | Observed Mass |
|---|---|---|---|---|---|---|
| Compound | R1 | R2 | R3 | R5 | Kd (μM) | (m/z) |
| ZZ′ | Et | Me | iPr | H | A (cIAP-1) | 508 |
| AAA | Me | Et | iPr | H | A (cIAP-1) | 508 |
| BBB | Me | Me | iPr | H | A | 494 |
| CCC | Et | Me | 2R—EtOH | H | A (cIAP-1) | 510.9 |
| DDD | Me | Et | 2R—EtOH | H | A (cIAP-1) | 510.9 |
| EEE | Me | Me | 2R—EtOH | H | A (cIAP-1) | 496 |
| FFF | Et | Me | CH₂OMe | H | B (cIAP-1) | 511 |
| GGG | Me | Et | CH₂OMe | H | B (cIAP-1) | 510 |
| HHH | Me | Me | CH₂OMe | H | B (cIAP-1) | 497 |
| III | Et | Me | Cyclohexyl | H | A (cIAP-1) | 549 |
| JJJ | Me | Et | Cyclohexyl | H | A (cIAP-1) | 535.1 |
| KKK | Me | Me | Cyclohexyl | H | B | 535 |
| LLL | Et | Me | 2R—EtOMe | H | A (cIAP-1) | 525 |
| MMM | Me | Et | 2R—EtOMe | H | A (cIAP-1) | 525 |
| NNN | Me | Me | 2R—EtOMe | H | A (cIAP-1) | 511 |

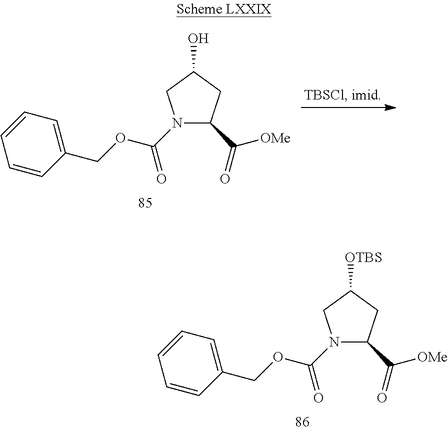

4-(tert-Butyl-dimethyl-silanyloxy)-pyrrolidine-1,2-dicarboxylic acid 1-benzyl ester 2-methyl ester (86): A solution of Z-Hyp-OMe (85, 49.4 g, 177 mmol) and imidazole (14.5 g, 214 mmol) were dissolved in DCM (215 mL) and cooled to 0° C. A solution containing tert-butyldimethylsilyl chloride (TBS-Cl, 29.8 g, 198 mmol) in DCM (100 mL) was added over about 68 minutes at ≦4° C. The reaction was allowed to warm and stir overnight at room temperature. TLC analysis indicated only a trace of starting material. The reaction was quenched with water (150 mL). The organic layer was washed with water (150 mL) containing conc. HCl (2-3 mL, pH was about 1) and then with brine (113 g). After concentration, the crude product (86) was obtained as an oil (93 g) which was used without further purification.

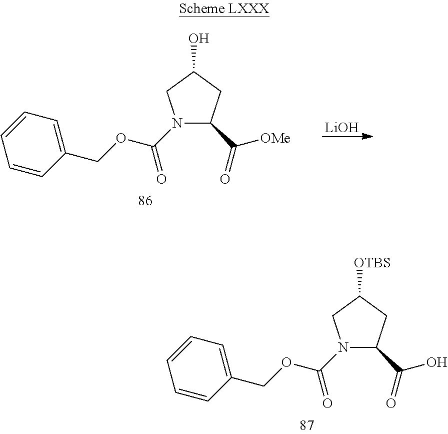

4-(tert-Butyl-dimethyl-silanyloxy)-pyrrolidine-1,2-dicarboxylic acid 1-benzyl ester (87): The oil from the previous step (86, 93 g, 177 mmol), THF (350 mL) and water (173 g) were combined and treated with LiOH monohydrate (7.8 g, 186 mmol) at room temperature. After 7 h, the reaction was complete by TLC analysis. The reaction mixture was diluted with water (350 mL) and extracted with isopropyl acetate (690 mL). The organic layer was extracted with water (170 mL). The combined aqueous layers were acidified with conc. HCl (19.7 g) to pH 2 and the product was extracted into toluene (350 mL). The organic layer was washed with water (350 mL) containing conc. HCl (1 g, pH 2). The organic layer was concentrated on the rotary evaporator and dried on a vacuum pump to provide a waxy solid (87, 62.9 g, 93%, two steps).

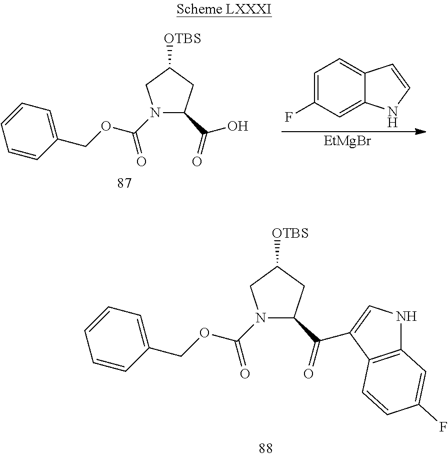

4-(tert-Butyl-dimethyl-silanyloxy)-2-(6-fluoro-1H-indole-3-carbonyl)-pyrrolidine-1-carboxylic acid benzyl ester (88): Z-Hyp(OTBS)-OH (87, 55.5 g, 145 mmol) was dissolved in toluene (265 mL). DMF (0.1 mL) and oxalyl chloride (22.4 g, 174 mmol) were added at room temperature. After 2-3 h, the bubbling stopped. After 4 h, the mixture was concentrated on a rotary evaporator (65° C. bath, ca. 30 min) to provide 95 g of a light yellow solution which was confirmed to be clean acid chloride with some traces of impurities present by ¹H NMR analysis.

6-Fluoroindole (39.2 g, 290 mmol) was dissolved in chlorobenzene (anhydrous, 300 mL) and toluene (200 mL) and the solution was cooled in an ice/acetone bath to −4° C. A solution of 3M EtMgBr in diethyl ether (101 g, 294 mmol) was added over 31 minutes at ≦2.5° C. resulting in a pale amber solution. After 30 min, the acid chloride/toluene solution from above was dripped in over about 45 minutes at <2° C. The reaction was kept cold for 1 h then let slowly warm. After about 4 h (10.6° C.), the reaction mixture was quenched with HOAc (9 g, exothermic to 17.5° C.) and then water (exothermic). A total of 200 mL water and 300 mL EtOAc were added. The organic layer was separated and washed with water (100 mL, slow separation). The organic layer was concentrated to afford 227 g of 88 as an amber-colored oil which was used without further purification.

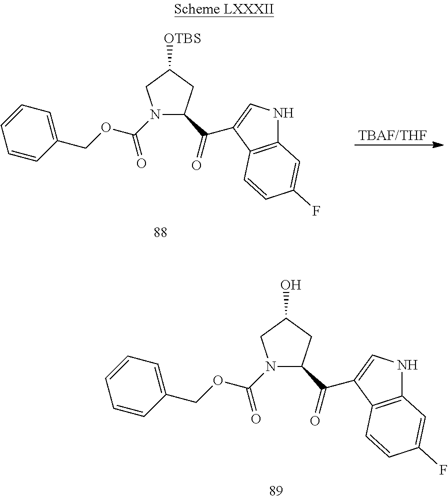

2-(6-Fluoro-1H-indole-3-carbonyl)-4-hydroxy-pyrrolidine-1-carboxylic acid benzyl ester (89): The oil from the previous step (88, 227 g) was diluted with THF (600 mL). A 1 M TBAF/THF solution (160 mL) was added and stirred at room temperature. After 9 h, another 20 mL of the 1 M TBAF/THF solution was added and the reaction was left over the weekend. The mixture was concentrated and redissolved in EtOAc (600 mL). Upon washing the solution with water (310 mL), the product precipitated to form a thick suspension. The mixture was filtered (slow) and the solids were washed with EtOAc (165 mL in portions) and dried to provide 43 g of 89 [77% overall yield for 2 steps based on Z-Hyp(OTBS)-OH]. The combined filtrates were concentrated to precipitate an additional 4.8 g (8.6%) of 89 after drying.

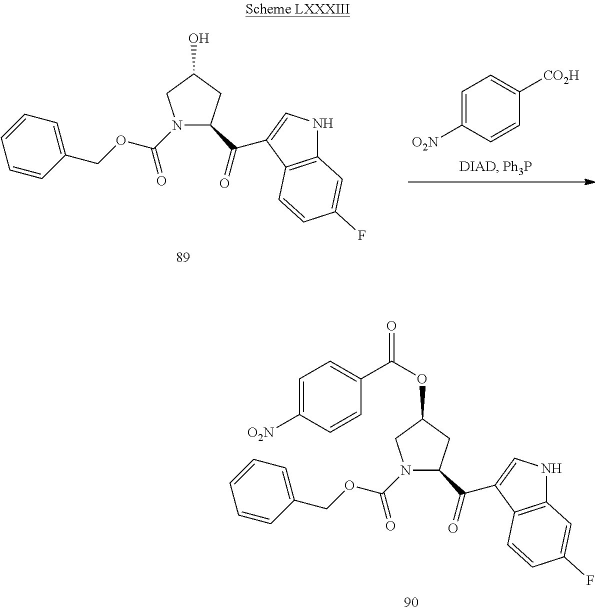

2-(6-Fluoro-1H-indole-3-carbonyl)-4-(4-nitro-benzoyloxy)-pyrrolidine-1-carboxylic acid benzyl ester (90): A solution containing 89 (51.1 g, 134 mmol), 4-nitrobenzoic acid (27.9 g, 167 mmol) and triphenylphosphine (48.9 g, 187 mmol) in anhydrous THF (700 mL) and DMF (175 mL) was cooled to 2° C. DIAD (37.4 mL, 194 mmol) was added over 1 h at 2-3° C. After 1 h, the solution was allowed to warm to room temperature and stir overnight. By HPLC analysis the reaction was complete. The reaction mixture was concentrated in vacuo and MeOH (250 mL) was added and concentrated to form a thick suspension (322 g). MeOH (250 mL) was again added and concentrated in vacuo to afford a thick suspension (420 g) that was chilled in an ice bath for about 1.5 h. The product was collected on a vacuum filter and washed with chilled MeOH (190 mL). The product air-dried on the filter to provide 82.9 g (>100%) of 90 as a light yellow-colored solid which still contained some residual MeOH.

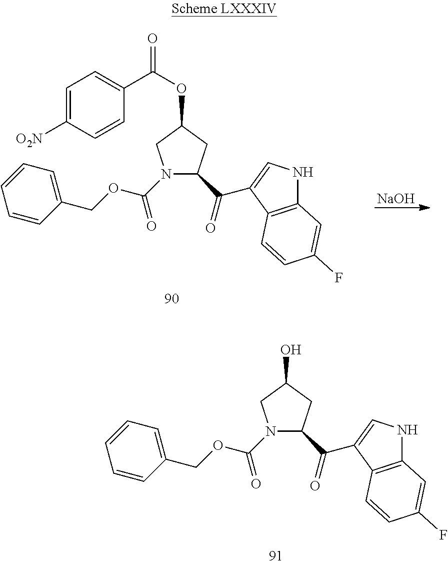

2-(6-Fluoro-1H-indole-3-carbonyl)-4-hydroxy-pyrrolidine-1-carboxylic acid benzyl ester (91): The damp solid from the previous step (90, 82.9 g) was suspended in a mixture of THF (600 mL), methanol (200 mL) and water (100 mL). A 50% aqueous NaOH solution (16.0 g, 200 mmol) was added (slightly exothermic from 23.7° C. to 25.9° C.). After 2 h, the reaction was complete by TLC analysis. HOAc (5.3 g) was added to adjust the pH to 7-8 (the orange solution color changed to pale yellowish) and the reaction mixture was concentrated in vacuo. Water (500 mL) was added and concentration was continued until a thick suspension formed (736 g). The product was collected on a vacuum filter and washed with water (400 mL in portions). The product was dried in a vacuum oven at 55° C. to provide 42.6 g (83%, 2 steps) of 91 as an off-white solid.

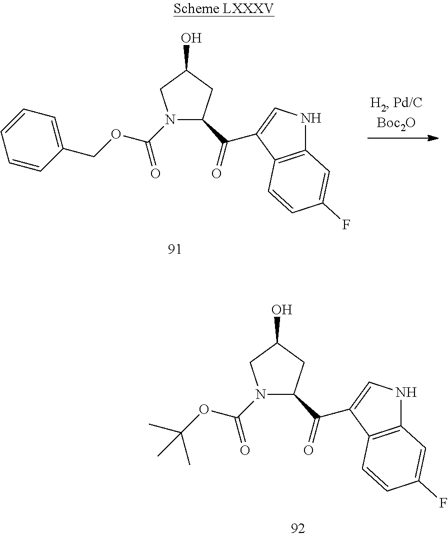

2-(6-Fluoro-1H-indole-3-carbonyl)-4-hydroxy-pyrrolidine-1-carboxylic acid tert-butyl ester (92): A suspension of 91 (3.8 g, 10 mmol), Boc₂O (2.4 g, 11 mmol), and 10% Pd-on-C (0.5 g, 5 mol %) in MeOH (50 mL) was shaken using a Parr apparatus at 40 PSI (2.72 atm) hydrogen pressure for 2 h. The reaction mixture was filtered and the filtrate was concentrated in vacuo to afford crude 92 as a white solid which was used without further purification. Mass spectrum, m/z=[348.7] (M)+.

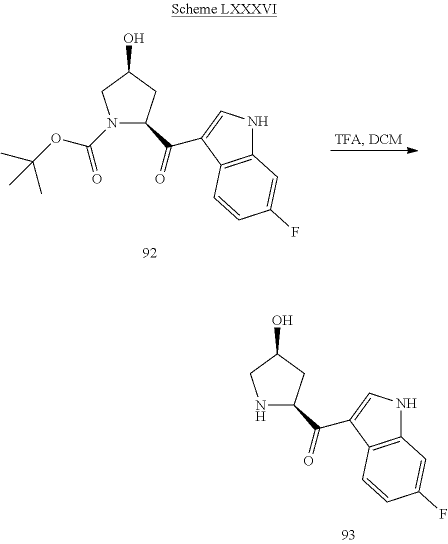

(6-Fluoro-1H-indol-3-yl)-(4-hydroxy-pyrrolidin-2-yl)-methanone (93): A solution containing crude 92 in DCM (20 mL) was cooled to 0° C. TFA (4 mL) was added. After 2 h, the reaction mixture was concentrated in vacuo and the crude product was purified by reverse-phase HPLC (2″ Dynamax C18 column; A: water w/0.1% v/v HOAc; B: ACN w/0.1% v/v HOAc; Method: 10-70% B over 30 min; Flow: 40 mL/min) to afford 2.3 g (95%, 2 steps) of 93 as a pale yellow foam following lyophilization. Mass spectrum, m/z=[248.7] (M)+.

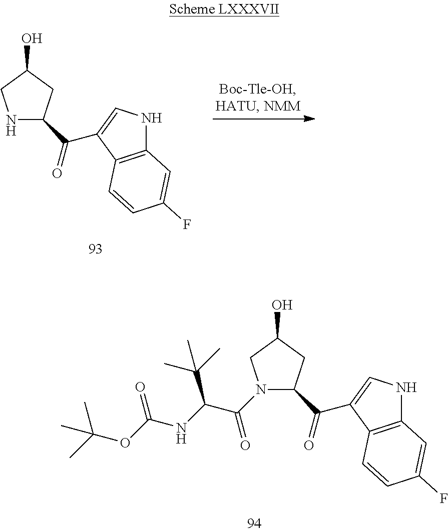

{1-[2-(6-Fluoro-1H-indole-3-carbonyl)-4-hydroxy-pyrrolidine-1-carbonyl]-2,2-dimethyl-propyl}-carbamic acid tert-butyl ester (94): To a solution containing amine 93 (0.30 g, 1.20 mmol), Boc-Tle-OH (0.31 g, 1.32 mmol), and HATU (0.50 g, 1.32 mmol) in NMP (13 mL) at 0° C. was added NMM (0.15 g, 1.44 mmol). The reaction mixture was allowed to warm to ambient temperature overnight. The reaction mixture was diluted with diethyl ether and washed successively with dilute aqueous HCl, water (5×), aqueous NaHCO₃, water (2×), then brine. The aqueous washes were back extracted with diethyl ether and the combined organic extracts were dried with anhydrous Na₂SO₄, filtered, and concentrated to afford the crude product which was purified by normal-phase HPLC (2″ Dynamax SiO₂ column (Varian, Inc.); A: hexanes; B: EtOAc; Method: 100% B over 30 min; Flow: 40 mL/min). The product-containing fractions were combined and concentrated in vacuo to afford 0.33 g (60%) of 94. Mass spectrum, m/z=[462.0] (M)+.

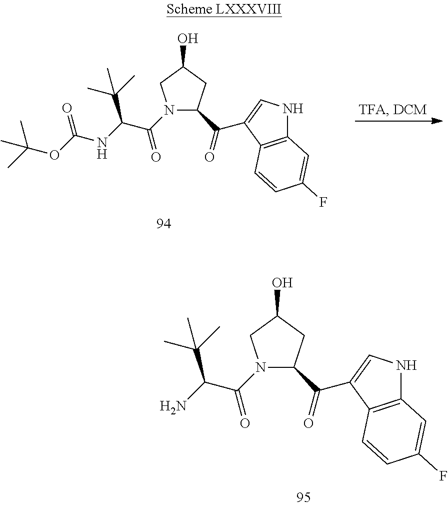

2-Amino-1-[2-(6-fluoro-1H-indole-3-carbonyl)-4-hydroxy-pyrrolidin-1-yl]-3,3-dimethyl-butan-1-one (95): A solution containing 94 (0.33 g, 0.72 mmol) in DCM (3 mL) was cooled to 0° C. TFA (1 mL) was added. After 2 h, the reaction mixture was concentrated in vacuo and the crude product was purified by reverse-phase HPLC (2″ Dynamax C18 column; A: water w/0.1% v/v HOAc; B: ACN w/0.1% v/v HOAc; Method: 10-70% B over 30 min; Flow: 40 mL/min) to afford 0.19 g (73%) of 95 following lyophilization. Mass spectrum, m/z=[361.8] (M)+.

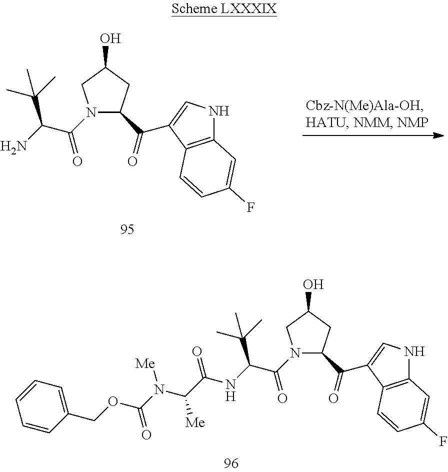

(1-{1-[2-(6-Fluoro-1H-indole-3-carbonyl)-4-hydroxy-pyrrolidine-1-carbonyl]-2,2-dimethyl-propylcarbamoyl}-ethyl)-methyl-carbamic acid benzyl ester (96): To a solution containing amine 95 (0.19 g, 0.53 mmol), Cbz-N(Me)Ala-OH (140 mg, 0.58 mmol), and HATU (220 mg, 0.58 mmol) in NMP (14 mL) at 0° C. was added NMM (60 mg, 0.64 mmol). The reaction mixture was allowed to warm to ambient temperature overnight. The reaction mixture was diluted with diethyl ether and washed successively with dilute aqueous HCl, water (5×), aqueous NaHCO₃, water (2×), then brine. The aqueous washes were back extracted with diethyl ether and the combined organic extracts were dried with anhydrous Na₂SO₄, filtered, and concentrated. The crude product was purified by reverse-phase HPLC (2″ Dynamax C18 column; A: water w/0.1% v/v HOAc; B: ACN w/0.1% v/v HOAc; Method: 30-100% B over 30 min; Flow: 40 mL/min) to afford 0.10 g (35%) of 96 following lyophilization. Mass spectrum, m/z=[581.0] (M)+.

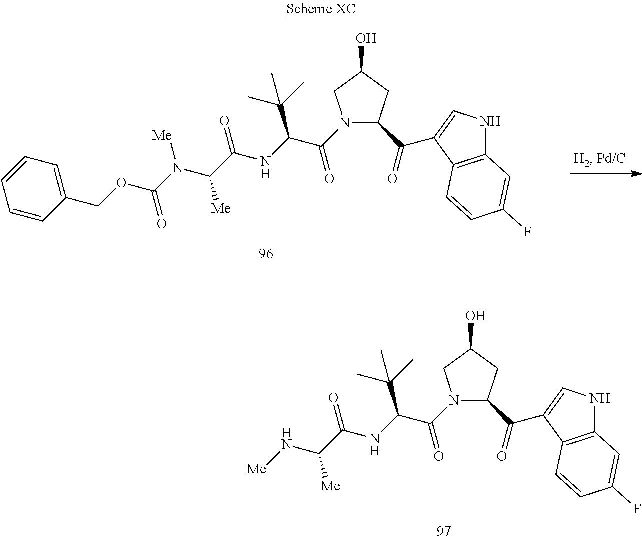

N-{1-[2-(6-Fluoro-1H-indole-3-carbonyl)-4-hydroxy-pyrrolidine-1-carbonyl]-2,2-dimethyl-propyl}-2-methylamino-propionamide (97): A solution containing 96 (0.1 g, 0.17 mmol) and 10% Pd-on-C (30 mg) in MeOH (20 mL) was shaken on a Parr apparatus under 45 PSI (3.06 atm) hydrogen pressure. After 2 h, the reaction mixture was filtered and concentrated. The crude product was purified by reverse-phase HPLC (2″ Dynamax C18 column; A: water w/0.1% v/v HOAc; B: ACN w/0.1% v/v HOAc; Method: 10-70% B over 30 min; Flow: 40 mL/min) to afford 69.4 mg (90%) of 97.HOAc following lyophilization. Mass spectrum, m/z=[447.0] (M)+.

Using the general procedures outlined in Schemes LXXIX through XC and the appropriate amino acid analogues to the amino acid reagents Boc-Tle-OH and Cbz-N(Me)Ala-OH, the compounds reported in Table 13 were prepared and tested for their binding affinities (Kd) to XIAP BIR-3 or cIAP-1 BIR-3.

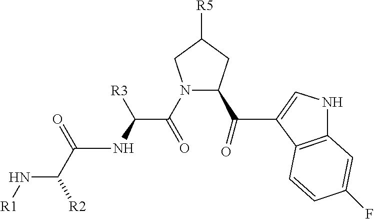

TABLE 13

| | | | | | | Observed |
|---|---|---|---|---|---|---|
| Com- | | | | | | Mass |
| pound | R1 | R2 | R3 | R5 | Kd (μM) | (m/z) |
| OOO | Me | Me | Cyclohexyl | (S)—OH | A (cIAP-1) | 473 |
| PPP | Me | Me | tert-Butyl | (S)—OH | A (cIAP-1) | 447.0 |
| QQQ | Me | Me | iPr | (S)—OH | A (cIAP-1) | 433 |
| RRR | Me | Me | Cyclohexyl | (R)—OH | C (cIAP-1) | 472.9 |
| SSS | Me | Me | tert-Butyl | (R)—OH | C (cIAP-1) | 447.0 |

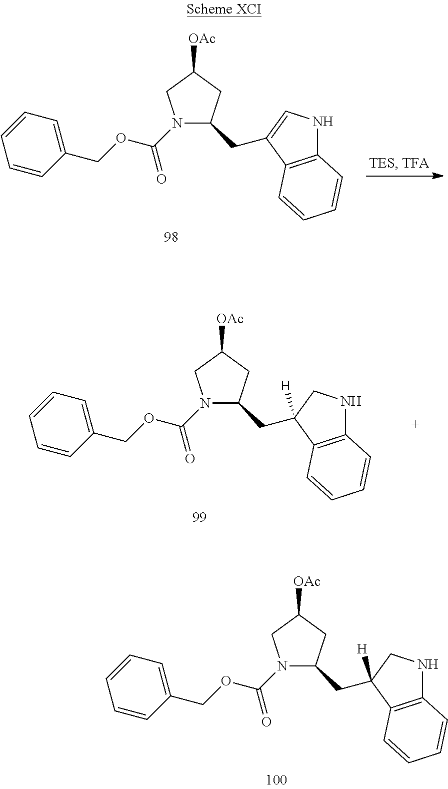

4-Acetoxy-2-(2,3-dihydro-1H-indol-3-ylmethyl)-pyrrolidine-1-carboxylic acid benzyl esters (99 and 100): TFA (100 mL) was cooled to 0° C. With vigorous stirring of the biphasic solution, triethylsilane (7.7 g, 66.5 mmol) was added in one portion followed by the dropwise addition of 98 (8.7 g, 22.1 mmol) in DCM (10 mL). After 2 h, the reaction mixture was concentrated in vacuo. The residue was dissolved in EtOAc and washed successively with saturated aqueous NaHCO₃ (until no gas evolution observed), then brine, dried over anhydrous Na₂SO₄, filtered, and concentrated. The crude products were purified by normal-phase HPLC (2″ Dynamax SiO₂, 10-100% EtOAc in hexanes over 30 min) to afford 6.5 g (75%) of an ˜1:1 mixture of 99 and 100 which was used directly in the next reaction.

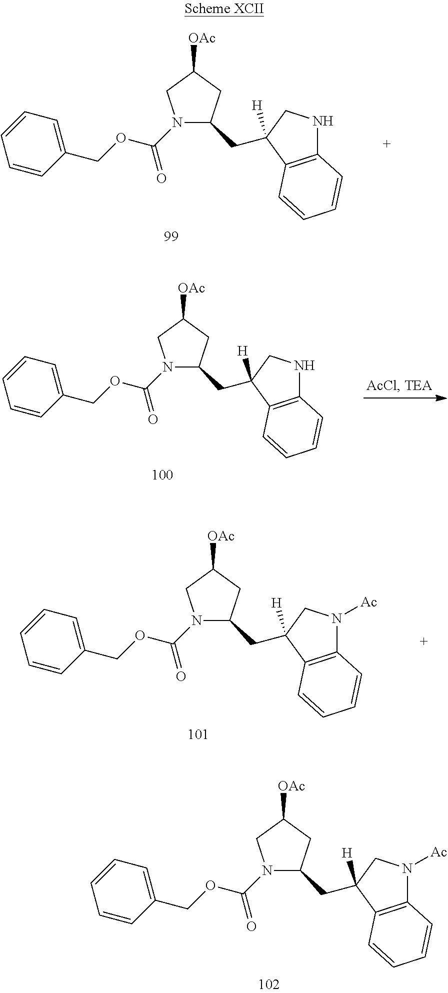

4-Acetoxy-2-(1-acetyl-2,3-dihydro-1H-indol-3-ylmethyl)-pyrrolidine-1-carboxylic acid benzyl esters (101 and 102): A solution containing ˜1:1 mixture of 99 and 100 (6.5 g, 16.4 mmol), TEA (2.5 g, 24.7 mmol), and DMAP (cat.) in DCM (100 mL) was cooled to 0° C. Acetylchloride (1.44 g, 18.1 mmol) was added via syringe. After 2 h, the heterogeneous reaction mixture was diluted with DCM and washed successively with aqueous NaHCO₃, water, and brine, dried over anhydrous Na₂SO₄, filtered, and concentrated. The crude products were purified by normal-phase HPLC (2″ Dynamax SiO₂, 34% EtOAc/hexanes) to afford 1.5 g (21%) of 101 and 2.8 g (39%) of 102. Mass spectrum, m/z=[436.6] (M)+.

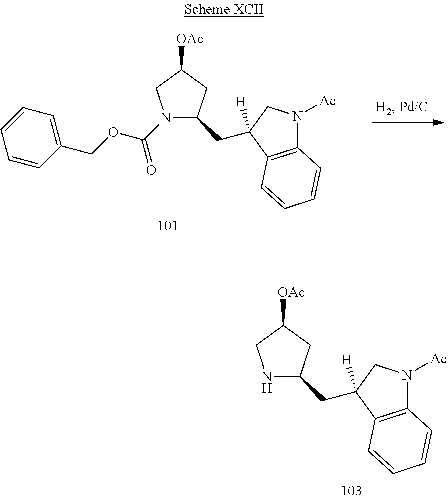

Acetic acid 5-(1-acetyl-2,3-dihydro-1H-indol-3-ylmethyl)-pyrrolidin-3-yl ester (103): A solution containing indoline 101 (0.2 g, 0.45 mmol) and 10% Pd-on-C (50 mg) in EtOAc (20 mL) was shaken on a Parr apparatus under 50 PSI (3.4 atm) hydrogen atmosphere. After 5 hr, the reaction mixture was filtered through Celite® and the solids were washed with EtOAc. The filtrate was concentrated to afford 0.26 g (>theory) of crude 103 which was used without further purification.

Using the general procedures outlined in Schemes XCI through XCIII and LXXXVIII through XC and the appropriate amino acid reagents, the compounds reported in Table 14 were prepared and tested for their binding affinities (Kd) to XIAP BIR-3 or cIAP-1 BIR-3.

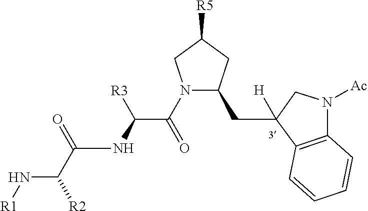

TABLE 14

| | | | | | Stereochemistry | | Observed Mass |
|---|---|---|---|---|---|---|---|
| Compound | R1 | R2 | R3 | R5 | at 3′ position | Kd (μM) | (m/z) |
| TTT | Me | Me | Cyclohexyl | OH | (S) | A (cIAP-1) | 484.7 |
| UUU | Me | Me | R—MeCHOMe | OH | (S) | A (cIAP-1) | 460.7 |
| VVV | Me | Me | Cyclohexyl | OH | (R) | A (cIAP-1) | 484.7 |
| WWW | Me | Me | R—MeCHOMe | OH | (R) | B (cIAP-1) | 460.7 |

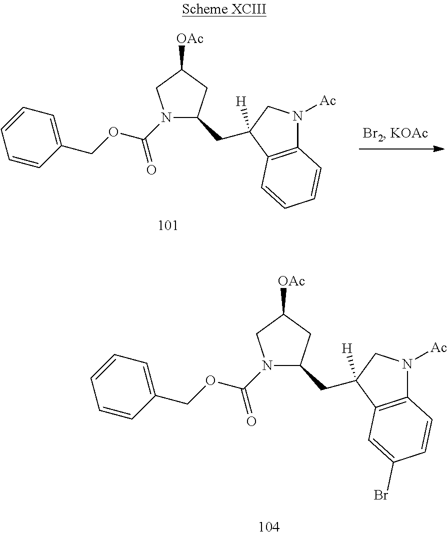

4-Acetoxy-2-(1-acetyl-5-bromo-2,3-dihydro-1H-indol-3-ylmethyl)-pyrrolidine-1-carboxylic acid benzyl ester (104): A solution containing 101 (0.8 g, 1.83 mmol) and KOAc (635 mg, 6.45 mmol) in CHCl₃ (30 mL) was cooled to 0° C. Bromine (0.35 g, 2.19 mmol) in CHCl₃ (5 mL) was added in a dropwise fashion. Following the addition of Br₂, LC/MS analysis revealed the presence of both 101 and 104, therefore an additional portion of KOAc (680 mg) and Br₂ (0.31 g in 5 mL CHCl₃) were added. Following the addition, the reaction was quenched by the addition of aqueous Na₂S₂O₃. The reaction mixture was diluted with DCM and the layers were separated. The organic phase was washed with brine, dried over anhydrous Na₂SO₄, filtered, and concentrated. The crude product was purified by normal-phase HPLC (2″ Dynamax SiO₂, 34% EtOAc/hexanes) to afford 104. Mass spectrum, m/z=[516.6] (M)+.

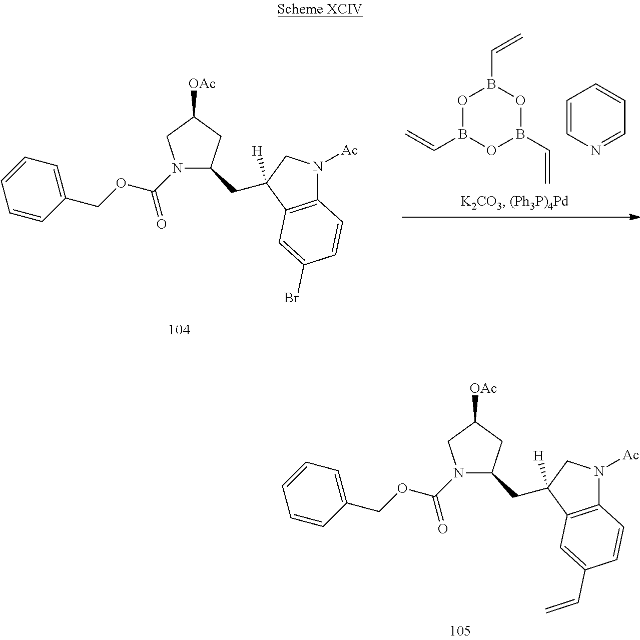

4-Acetoxy-2-(1-acetyl-5-vinyl-2,3-dihydro-1H-indol-3-ylmethyl)-pyrrolidine-1-carboxylic acid benzyl ester (105): A mixture containing 104 (0.32 g, 0.62 mmol), (Ph₃P)₄Pd (7 mg, 0.01 mol %), 2,4,6-trivinylcycloboroxane pyridine complex (150 mg, 0.62 mmol), K₂CO₃ (86 mg, 0.62 mmol) in 4:1 DME/water was warmed to 90° C. After 8 h, the reaction mixture was cooled and diluted with EtOAc. The organic solution was washed successively with water and brine, dried over anhydrous Na₂SO₄, filtered, and concentrated. The crude product was combined with the crude product from a second reaction performed on 0.35 mmol-scale and purified by normal-phase HPLC (2″ Dynamax SiO₂, 60-100% EtOAc in hexanes over 30 min) to afford 260 mg (59%) of 105. Mass spectrum, m/z=[462.6] (M)+.

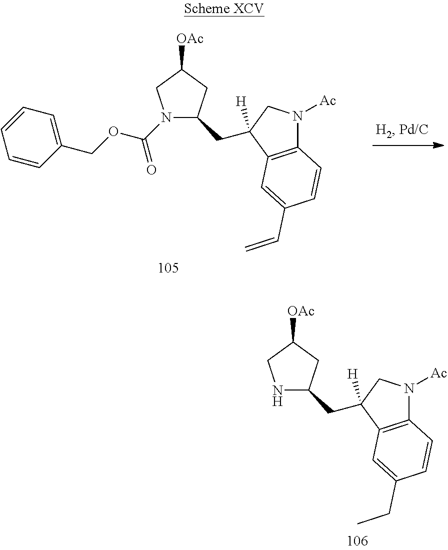

Acetic acid 5-(1-acetyl-5-ethyl-2,3-dihydro-1H-indol-3-ylmethyl)-pyrrolidin-3-yl ester (106): A solution containing indoline 105 (0.26 g, 0.56 mmol) and 10% Pd-on-C (100 mg) in EtOAc (20 mL) was shaken on a Parr apparatus under 50 PSI (3.4 atm) hydrogen atmosphere. After 8 h, the reaction mixture was filtered through Celite® and the solids were washed with EtOAc. The filtrate was concentrated to afford 0.26 g (>theory) of crude 106 which was used without further purification. Mass spectrum, m/z=[330.6] (M)+.

Using the general procedures outlined in Schemes XCIII through XCV and LXXXVIII through XC and the appropriate amino acid reagents, the compounds reported in Table 15 were prepared and tested for their binding affinities (Kd) to XIAP BIR-3 or cIAP-1 BIR-3.

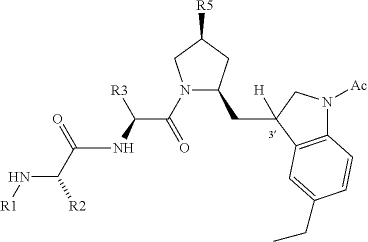

TABLE 15

| | | | | | | | Observed |
|---|---|---|---|---|---|---|---|
| | | | | | Stereochemistry | | Mass |
| Compound | R1 | R2 | R3 | R5 | at 3′ position | Kd (μM) | (m/z) |
| XXX | Me | Me | Cyclohexyl | OH | (S) | A (cIAP-1) | 511.1 |
| YYY | Et | Me | Cyclohexyl | OH | (S) | B (cIAP-1) | 526.2 |
| ZZZ | Me | Me | R—MeCHOMe | OH | (S) | A (cIAP-1) | 489.1 |
| AAAA | Et | Me | R—MeCHOMe | OH | (R) | B (cIAP-1) | 504.1 |
| BBBB | Me | Me | Cyclohexyl | OH | (R) | B (cIAP-1) | 512.8 |
| CCCC | Et | Me | Cyclohexyl | OH | (R) | B (cIAP-1) | 527.2 |
| DDDD | Me | Me | R—MeCHOMe | OH | (R) | A (cIAP-1) | 489.1 |
| EEEE | Et | Me | R—MeCHOMe | OH | (R) | B (cIAP-1) | 504.1 |

The compounds of the present invention may exist in unsolvated forms as well as solvated forms, including hydrated forms. The compounds of the present invention (e.g., compounds of Formula I) also are capable of forming both pharmaceutically acceptable salts, including but not limited to acid addition and/or base salts. Furthermore, compounds of the present invention may exist in an amorphous form (noncrystalline form), and in the form of clathrates, prodrugs, polymorphs, bio-hydrolyzable esters, racemic mixtures, or as purified stereoisomers including, but not limited to, optically pure enantiomers and diastereomers. In general, all of these forms can be used as an alternative form to the free base or acid forms of the compounds, as described above and are intended to be encompassed within the scope of the present invention.

A “polymorph” refers to solid crystalline forms of a compound. Different polymorphs of the same compound can exhibit different physical, chemical and/or spectroscopic properties. Different physical properties include, but are not limited to stability (e.g., to heat or light), compressibility and density (important in formulation and product manufacturing), and dissolution rates (which can affect bioavailability). Different physical properties of polymorphs can affect their processing. A “clathrate” means a compound or a salt thereof in the form of a crystal lattice that contains spaces (e.g., channels) that have a guest molecule (e.g., a solvent or water) trapped within. The term “prodrug” refers to compounds that are rapidly transformed in vivo to yield the parent compound of the above formulae, for example, by hydrolysis in blood. A thorough discussion is provided in T. Higuchi and V. Stella, “Pro-drugs as Novel Delivery Systems,” Vol 14 of the A.C.S. Symposium Series, and in Bioreversible Carriers in Drug Design, ed. Edward B. Roche, American Pharmaceutical Association and Pergamon Press, 1987, both of which are incorporated herein by reference.

Compounds and salts of the present invention may also exist in tautomeric forms, such as an enol and an imine form, and the corresponding keto and enamine forms and geometric isomers and mixtures thereof. Tautomers exist as mixtures of a tautomeric set in solution. In solid form, usually one tautomer predominates. Even though only one tautomer may be described by the formulae above, the present invention includes all tautomers of the present compounds.

The compounds of the present invention can be administered to a patient either alone or a part of a pharmaceutical composition. A variety of non-limiting methods for administering the compounds and related compositions to patients include orally, rectally, parenterally (intravenously, intramuscularly, or subcutaneously), intracisternally, intravaginally, intraperitoneally, intravesically, locally (powders, ointments, or drops), or as a buccal or nasal spray.

Pharmaceutical compositions to be used comprise a therapeutically effective amount of a compound as described above, or a pharmaceutically acceptable salt or other form thereof together with a pharmaceutically acceptable excipient. The phrase “pharmaceutical composition” refers to a composition suitable for administration in medical or veterinary use. It should be appreciated that the determinations of proper dosage forms, dosage amounts, and routes of administration are within the level of ordinary skill in the pharmaceutical and medical arts.

Compositions suitable for parenteral administration conveniently comprise a sterile aqueous preparation of a compound or composition of the invention, which is preferably isotonic with the blood of the recipient. This aqueous preparation may be formulated according to known methods using suitable dispersing or wetting agents, emulsifying and suspending agents. Various antibacterial and antifungal agents, for example, parabens, chlorobutanol, phenol, and sorbic acid also may be included. The sterile injectable preparation also may be a sterile injectable solution or suspension in a non-toxic parenterally-acceptable diluent or solvent, for example, as a solution in 1,3-butane diol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed including synthetic mono- or di-glycerides. In addition, fatty acids such as oleic acid may be used in the preparation of injectables. Prolonged absorption of the injectable pharmaceutical form can be brought about by the use of agents delaying absorption, for example, aluminum monostearate and gelatin. Carrier formulation suitable for subcutaneous, intravenous, intramuscular, etc. administrations can be found in Remington's Pharmaceutical Sciences, Mack Publishing Co., Easton, Pa. which is incorporated herein in its entirety by reference thereto.

Solid dosage forms for oral administration include capsules, tablets, pills, powders, and granules. In such solid dosage forms, the compound is admixed with at least one inert pharmaceutically acceptable excipient such as (a) fillers or extenders, as for example, starches, lactose, sucrose, glucose, mannitol, and silicic acid, (b) binders, as for example, carboxymethylcellulose, alignates, gelatin, polyvinylpyrrolidone, sucrose, and acacia, (c) humectants, as for example, glycerol, (d) disintegrating agents, as for example, agar-agar, calcium carbonate, potato or tapioca starch, alginic acid, certain complex silicates, and sodium carbonate, (e) solution retarders, as for example paraffin, (f) absorption accelerators, as for example, quaternary ammonium compounds, (g) wetting agents, as for example, cetyl alcohol, and glycerol monostearate, (h) adsorbents, as for example, kaolin and bentonite, and (i) lubricants, as for example, talc, calcium stearate, magnesium stearate, solid polyethylene glycols, sodium lauryl sulfate, or mixtures thereof. In the case of capsules, tablets, and pills, the dosage forms may also comprise buffering agents. Solid dosage forms such as tablets, dragees, capsules, pills, and granules also can be prepared with coatings and shells, such as enteric coatings and others well known in the art. The solid dosage form also may contain opacifying agents, and can also be of such composition that they release the active compound or compounds in a certain part of the intestinal tract in a delayed manner. Examples of embedding compositions which can be used are polymeric substances and waxes. The active compounds can also be in micro-encapsulated form, if appropriate, with one or more of the above-mentioned excipients. Such solid dosage forms may generally contain from 1% to 95% (w/w) of the active compound. In certain embodiments, the active compound ranges from 5% to 70% (w/w).

Liquid dosage forms for oral administration include pharmaceutically acceptable emulsions, solutions, suspensions, syrups, and elixirs. In addition to the compound or composition, the liquid dosage forms may contain inert diluents commonly used in the art, such as water or other solvents, solubilizing agents and emulsifiers, as for example, ethyl alcohol, isopropyl alcohol, ethyl carbonate, ethyl acetate, benzyl alcohol, benzyl benzoate, propyleneglycol, 1,3-butyleneglycol, dimethylformamide, oils, in particular, cottonseed oil, groundnut oil, corn germ oil, olive oil, castor oil and sesame oil, glycerol, tetrahydrofurfuryl alcohol, polyethyleneglycols and fatty acid esters of sorbitan or mixtures of these substances. Besides such inert diluents, the composition can also include adjuvants, such as wetting agents, emulsifying and suspending agents, sweetening, flavoring, and perfuming agents.

Compositions for rectal administrations are preferably suppositories which can be prepared by mixing compounds of the present invention with suitable non-irritating excipients or carriers such as cocoa butter, polyethyleneglycol or a low-melting, suppository wax, which are solid at ordinary temperatures but liquid at body temperature and therefore, melt in the rectum or vaginal cavity and release the active compound.

Dosage forms for topical administration of a compound of this invention include ointments, powders, sprays, and inhalants. The active compound is admixed under sterile conditions with a physiologically acceptable carrier and any preservatives, buffers, or propellants as may be required. Ophthalmic formulations, eye ointments, powders, and solutions are also contemplated as being within the scope of this invention.

The compounds and compositions of the present invention also may benefit from a variety of delivery systems, including time-released, delayed release or sustained release delivery systems. Such option may be particularly beneficial when the compounds and composition are used in conjunction with other treatment protocols as described in more detail below.

Many types of release delivery systems are available and known to those of ordinary skill in the art. They include polymer base systems such as poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109. Delivery systems also include non-polymer systems that are: lipids including sterols such as cholesterol, cholesterol esters and fatty acids or neutral fats such as mono-di- and tri-glycerides; hydrogel release systems; sylastic systems; peptide based systems; wax coatings; compressed tablets using conventional binders and excipients; partially fused implants; and the like. Specific examples include, but are not limited to: (a) erosional systems in which the active compound is contained in a form within a matrix such as those described in U.S. Pat. Nos. 4,452,775, 4,667,014, 4,748,034 and 5,239,660 and (b) diffusional systems in which an active component permeates at a controlled rate from a polymer such as described in U.S. Pat. Nos. 3,832,253, and 3,854,480. In addition, pump-based hardware delivery systems can be used, some of which are adapted for implantation.

Use of a long-term sustained release implant may be desirable. Long-term release, as used herein, means that the implant is constructed and arranged to deliver therapeutic levels of the active compound for at least 30 days, and preferably 60 days. Long-term sustained release implants are well-known to those of ordinary skill in the art and include some of the release systems described above.

In practicing the methods of the present invention, the compounds and compositions of the present invention are administered in a therapeutically effective amount. Generally, doses of active compounds would be from about 0.01 mg/kg per day to 1000 mg/kg per day. It is expected that doses ranging from 50-500 mg/kg will be suitable, preferably intravenously, intramuscularly, or intradermally, and in one or several administrations per day. When practicing the conjoint or combination therapy described in more detail below, the administration of the compounds and compositions of the present invention can occur simultaneous with, subsequent to, or prior to chemotherapy or radiation, so long as the chemotherapeutic agent or radiation sensitizes the system to the compounds and compositions of the present invention.

In general, routine experimentation in clinical trials will determine specific ranges for optimal therapeutic effect for a particular compound and composition of the present invention and each administrative protocol, and administration to specific patients will be adjusted to within effective and safe ranges depending on the patient condition and responsiveness to initial administrations. However, the ultimate administration protocol will be regulated according to the judgment of the attending clinician considering such factors as age, condition and size of the patient, the potency of the compound or composition, the duration of the treatment and the severity of the disease being treated. For example, a dosage regimen of the compound or composition can be an oral administration of from 1 mg to 2000 mg/day, preferably 1 to 1000 mg/day, more preferably 50 to 600 mg/day, in two to four (preferably two) divided doses, to reduce tumor growth. Intermittent therapy (e.g., one week out of three weeks or three out of four weeks) may also be used.

In the event that a response in a subject is insufficient at the initial doses applied, higher doses (or effectively higher doses by a different, more localized delivery route) may be employed to the extent that the patient tolerance permits. Multiple doses per day are contemplated to achieve appropriate systemic levels of compounds. Generally, a maximum dose is used, that is, the highest safe dose according to sound medical judgment. Those of ordinary skill in the art will understand, however, that a patient may insist upon a lower dose or tolerable dose for medical reasons, psychological reasons or for virtually any other reason.

The compounds of the present invention and pharmaceutical compositions comprising a compound of the present invention can be administered to a subject suffering from cancer, an autoimmune disease or another disorder where a defect in apoptosis is implicated. In connection with such treatments, the patient can be treated prophylactically, acutely, or chronically using compounds and compositions of the present invention, depending on the nature of the disease. Typically, the host or subject in each of these methods is human, although other mammals may also benefit from the administration of a compound of the present invention.

As described in U.S. Pat. No. 7,244,851, the disclosure of which is incorporated herein by reference, TAP antagonists can be used for the treatment of all cancer types which fail to undergo apoptosis. Thus, compounds of the present invention can be used to provide a therapeutic approach to the treatment of many kinds of solid tumors, including but not limited to carcinomas, sarcomas including Kaposi's sarcoma, erythroblastoma, glioblastoma, meningioma, astrocytoma, melanoma and myoblastoma. Treatment or prevention of non-solid tumor cancers such as leukemia is also contemplated by this invention. Indications may include, but are not limited to brain cancers, skin cancers, bladder cancers, ovarian cancers, breast cancers, gastric cancers, pancreatic cancers, colon cancers, blood cancers, lung cancers and bone cancers. Examples of such cancer types include neuroblastoma, intestine carcinoma such as rectum carcinoma, colon carcinoma, familiary adenomatous polyposis carcinoma and hereditary non-polyposis colorectal cancer, esophageal carcinoma, labial carcinoma, larynx carcinoma, hypopharynx carcinoma, tong carcinoma, salivary gland carcinoma, gastric carcinoma, adenocarcinoma, medullary thyroidea carcinoma, papillary thyroidea carcinoma, renal carcinoma, kidney parenchym carcinoma, ovarian carcinoma, cervix carcinoma, uterine corpus carcinoma, endometrium carcinoma, chorion carcinoma, pancreatic carcinoma, prostate carcinoma, testis carcinoma, breast carcinoma, urinary carcinoma, melanoma, brain tumors such as glioblastoma, astrocytoma, meningioma, medulloblastoma and peripheral neuroectodermal tumors, Hodgkin lymphoma, non-Hodgkin lymphoma, Burkitt lymphoma, acute lymphatic leukemia (ALL), chronic lymphatic leukemia (CLL), acute myeloid leukemia (AML), chronic myeloid leukemia (CML), adult T-cell leukemia lymphoma, hepatocellular carcinoma, gall bladder carcinoma, bronchial carcinoma, small cell lung carcinoma, non-small cell lung carcinoma, multiple myeloma, basalioma, teratoma, retinoblastoma, choroidea melanoma, seminoma, rhabdomyo sarcoma, craniopharyngeoma, osteosarcoma, chondrosarcoma, myosarcoma, liposarcoma, fibrosarcoma, Ewing sarcoma and plasmocytoma.

The inventors believe that the IAP antagonists of the present invention will be particularly active for treating human malignancies where cIAP1 and cIAP2 are over-expressed (e.g., lung cancers, see Dai et al, Hu. Molec. Genetics, 2003 v 12 pp 791-801; leukemias (multiple references), and other cancers (Tamm et al, Clin Cancer Res, 2000, v 6, 1796-1803). The inventors also expect that the IAP antagonists of the present invention will be active in disorders that may be driven by inflammatory cytokines such as TNF playing a pro-survival role (for example, there is a well defined role for TNF acting as a survival factor in ovarian carcinoma, similarly for gastric cancers (see Kulbe, et al, Cancer Res 2007, 67, 585-592).

In addition to apoptosis defects found in tumors, defects in the ability to eliminate self-reactive cells of the immune system due to apoptosis resistance are considered to play a key role in the pathogenesis of autoimmune diseases. Autoimmune diseases are characterized in that the cells of the immune system produce antibodies against its own organs and molecules or directly attack tissues resulting in the destruction of the latter. A failure of those self-reactive cells to undergo apoptosis leads to the manifestation of the disease. Defects in apoptosis regulation have been identified in autoimmune diseases such as systemic lupus erythematosus or rheumatoid arthritis.

Examples of such autoimmune diseases include collagen diseases such as rheumatoid arthritis, systemic lupus erythematosus, Sharp's syndrome, CREST syndrome (calcinosis, Raynaud's syndrome, esophageal dysmotility, telangiectasia), dermatomyositis, vasculitis (Morbus Wegener's) and Sjögren's syndrome, renal diseases such as Goodpasture's syndrome, rapidly-progressing glomerulonephritis and membrano-proliferative glomerulonephritis type II, endocrine diseases such as type-I diabetes, autoimmune polyendocrinopathy-candidiasis-ectodermal dystrophy (APECED), autoimmune parathyroidism, pernicious anemia, gonad insufficiency, idiopathic Morbus Addison's, hyperthyreosis, Hashimoto's thyroiditis and primary myxedema, skin diseases such as pemphigus vulgaris, bullous pemphigoid, herpes gestationis, epidermolysis bullosa and erythema multiforme major, liver diseases such as primary biliary cirrhosis, autoimmune cholangitis, autoimmune hepatitis type-1, autoimmune hepatitis type-2, primary sclerosing cholangitis, neuronal diseases such as multiple sclerosis, myasthenia gravis, myasthenic Lambert-Eaton syndrome, acquired neuromyotony, Guillain-Barré syndrome (Müller-Fischer syndrome), stiff-man syndrome, cerebellar degeneration, ataxia, opsoklonus, sensoric neuropathy and achalasia, blood diseases such as autoimmune hemolytic anemia, idiopathic thrombocytopenic purpura (Morbus Werlhof), infectious diseases with associated autoimmune reactions such as AIDS, Malaria and Chagas disease.

The present invention also is directed to the use of the compounds and compositions as a chemopotentiating agent with other treatment approaches. The term “chemopotentiating agent” refers to an agent that acts to increase the sensitivity of an organism, tissue, or cell to a chemical compound, or treatment namely “chemotherapeutic agents” or “chemo drugs” or to radiation treatment. Thus, compounds and compositions of the present invention can be used for inhibiting tumor growth in vivo by administering them in combination with a biologic or chemotherapeutic agent or by using them in combination with chemoradiation. In these applications, the administration of the compounds and compositions of the present invention may occur prior to, and with sufficient time, to cause sensitization of the site to be treated. Alternatively, the compounds and compositions of the present invention may be used contemporaneously with radiation and/or additional anti-cancer chemical agents (infra). Such systems can avoid repeated administrations of the compounds and compositions of the present invention, increasing convenience to the subject and the physician, and may be particularly suitable for certain compositions of the present invention.

Biological and chemotherapeutics/anti-neoplastic agents and radiation induce apoptosis by activating the extrinsic or intrinsic apoptotic pathways, and, since the compounds and compositions of the present invention relieve inhibitors of apoptotic proteins (IAPs) and, thus, remove the block in apoptosis, the combination of chemotherapeutics/anti-neoplastic agents and radiation with the compounds and compositions of the present invention should work synergistically to facilitate apoptosis.

A combination of a compound of the present invention and a chemotherapeutic/anti neoplastic agent and/or radiation therapy of any type that activates the intrinsic pathway may provide a more effective approach to destroying tumor cells. Compounds of the present invention interact with IAP's, such as XIAP, cIAP-1, cIAP-2, ML-IAP, etc., and block the IAP mediated inhibition of apoptosis while chemotherapeutics/anti neoplastic agents and/or radiation therapy kills actively dividing cells by activating the intrinsic apoptotic pathway leading to apoptosis and cell death. As is described in more detail below, embodiments of the invention provide combinations of a compound of the present invention and a chemotherapeutic/anti-neoplastic agent and/or radiation which provide a synergistic action against unwanted cell proliferation. This synergistic action between a compound of the present invention and a chemotherapeutic/anti-neoplastic agent and/or radiation therapy can improve the efficiency of the chemotherapeutic/anti-neoplastic agent and/or radiation therapies. This will allow for an increase in the effectiveness of current chemotherapeutic/anti-neoplastic agents or radiation treatments allowing the dose of the chemotherapeutic/anti-neoplastic agent to be lowered, therein providing both a more effective dosing schedule as well as use of a more tolerable dose of chemotherapeutic/anti-neoplastic agent and/or radiation.

In an embodiment of the present invention, the patient is treated by administering a compound or a pharmaceutical composition of the present invention at a time the patient is subject to concurrent or antecedent radiation or chemotherapy for treatment of a neoproliferative pathology of a tumor such as, but not limited to, bladder cancer, breast cancer, prostate cancer, lung cancer, pancreatic cancer, gastric cancer, colon cancer, ovarian cancer, renal cancer, hepatoma, melanoma, lymphoma, sarcoma, and combinations thereof.

In another embodiment of the present invention, the compound or composition of the present invention can be administered in combination with a chemotherapeutic and/or for use in combination with radiotherapy, immunotherapy, and/or photodynamic therapy, promoting apoptosis and enhancing the effectiveness of the chemotherapeutic, radiotherapy, immunotherapy, and/or photodynamic therapy.

Embodiments of the invention also include a method of treating a patient afflicted with cancer by the contemporaneous or concurrent administration of a chemotherapeutic agent. Such chemotherapeutic agents include but are not limited to the chemotherapeutic agents described in “Modern Pharmacology with Clinical Applications”, Sixth Edition, Craig & Stitzel, Chpt. 56, pg 639-656 (2004), herein incorporated by reference. The chemotherapeutic agent can be, but is not limited to, alkylating agents, antimetabolites, anti-tumor antibiotics, plant-derived products such as taxanes, enzymes, hormonal agents, miscellaneous agents such as cisplatin, monoclonal antibodies, glucocorticoids, mitotic inhibitors, topoisomerase I inhibitors, topoisomerase II inhibitors, immunomodulating agents such as interferons, cellular growth factors, cytokines, and nonsteroidal anti-inflammatory compounds, cellular growth factors and kinase inhibitors. Other suitable classifications for chemotherapeutic agents include mitotic inhibitors and nonsteroidal anti-estrogenic analogs.

Specific examples of suitable biological and chemotherapeutic agents include, but are not limited to, cisplatin, carmustine (BCNU), 5-fluorouracil (5-FU), cytarabine (Ara-C), gemcitabine, methotrexate, daunorubicin, doxorubicin, dexamethasone, topotecan, etoposide, paclitaxel, vincristine, tamoxifen, TNF-alpha, TRAIL, interferon (in both its alpha and beta forms), thalidomide, and melphalan. Other specific examples of suitable chemotherapeutic agents include nitrogen mustards such as cyclophosphamide, alkyl sulfonates, nitrosoureas, ethylenimines, triazenes, folate antagonists, purine analogs, pyrimidine analogs, anthracyclines, bleomycins, mitomycins, dactinomycins, plicamycin, vinca alkaloids, epipodophyllotoxins, taxanes, glucocorticoids, L-asparaginase, estrogens, androgens, progestins, luteinizing hormones, octreotide actetate, hydroxyurea, procarbazine, mitotane, hexamethylmelamine, carboplatin, mitoxantrone, monoclonal antibodies, levamisole, interferons, interleukins, filgrastim and sargramostim. Chemotherapeutic compositions also comprise other members, i.e., other than TRAIL, of the TNF superfamily of compounds.

Another embodiment of the present invention relates to the use of a compound or composition of the present invention in combination with topoisomerase inhibitors to potentiate their apoptotic inducing effect. Topoisomerase inhibitors inhibit DNA replication and repair, thereby promoting apoptosis and have been used as chemothemotherapeutic agents. Topoisomerase inhibitors promote DNA damage by inhibiting the enzymes that are required in the DNA repair process. Therefore, export of Smac from the mitochondria into the cell cytosol is provoked by the DNA damage caused by topoisomerase inhibitors. Topoisomerase inhibitors of both the Type I class (camptothecin, topotecan, SN-38 (irinotecan active metabolite)) and the Type II class (etoposide) are expected to show potent synergy with compounds of the present invention. Further examples of topoisomerase inhibiting agents that may be used include, but are not limited to, irinotecan, topotecan, etoposide, amsacrine, exatecan, gimatecan, etc. Other topoisomerase inhibitors include, for example, Aclacinomycin A, camptothecin, daunorubicin, doxorubicin, ellipticine, epirubicin, and mitaxantrone.

In another embodiment of the invention, the chemotherapeutic/anti-neoplastic agent for use in combination with the compounds and compositions of the present invention may be a platinum containing compound. In one embodiment of the invention, the platinum containing compound is cisplatin. Cisplatin can synergize with a compound of the present invention and potentiate the inhibition of an IAP, such as but not limited to XIAP, cIAP-1, c-IAP-2, ML-IAP, etc. In another embodiment a platinum containing compound is carboplatin. Carboplatin can synergize with a compound of the present invention and potentiate the inhibition of an IAP, including, but not limited to, XIAP, cIAP-1, c-IAP-2, ML-IAP, etc. In another embodiment a platinum containing compound is oxaliplatin. The oxaliplatin can synergize with a compound of the present invention and potentiate the inhibition of an IAP, including, but not limited to, XIAP, cIAP-1, c-IAP-2, ML-IAP, etc.

Platinum chemotherapy drugs belong to a general group of DNA modifying agents. DNA modifying agents may be any highly reactive chemical compound that bonds with various nucleophilic groups in nucleic acids and proteins and cause mutagenic, carcinogenic, or cytotoxic effects. DNA modifying agents work by different mechanisms, disruption of DNA function and cell death; DNA damage/the formation of cross-bridges or bonds between atoms in the DNA; and induction of mispairing of the nucleotides leading to mutations, to achieve the same end result. Three non-limiting examples of a platinum containing DNA modifying agents are cisplatin, carboplatin and oxaliplatin.

Cisplatin is believed to kill cancer cells by binding to DNA and interfering with its repair mechanism, eventually leading to cell death. Carboplatin and oxaliplatin are cisplatin derivatives that share the same mechanism of action. Highly reactive platinum complexes are formed intracellularly and inhibit DNA synthesis by covalently binding DNA molecules to form intrastrand and interstrand DNA crosslinks.

Non-steroidal anti-inflammatory drugs (NSAIDs) have been shown to induce apoptosis in colorectal cells. NSAIDs appear to induce apoptosis via the release of Smac from the mitochondria (PNAS, Nov. 30, 2004, vol. 101:16897-16902). Therefore, the use of NSAIDs in combination with the compounds and compositions of the present invention would be expected to increase the activity of each drug over the activity of either drug independently.

Many naturally occurring compounds isolated from bacterial, plant, and animals can display potent and selective biological activity in humans including anticancer and antineoplastic activities. In fact, many natural products, or semi-synthetic derivatives thereof, which possess anticancer activity, are already commonly used as therapeutic agents; these include paclitaxel, etoposide, vincristine, and camptothecin amongst others. Additionally, there are many other classes of natural products such as the indolocarbazoles and epothilones that are undergoing clinical evaluation as anticancer agents. A reoccurring structural motif in many natural products is the attachment of one or more sugar residues onto an aglycone core structure. In some instances, the sugar portion of the natural product is critical for making discrete protein-ligand interactions at its site of action (i.e., pharmacodynamics) and removal of the sugar residue results in significant reductions in biological activity. In other cases, the sugar moiety or moieties are important for modulating the physical and pharmacokinetic properties of the molecule. Rebeccamycin and staurosporine are representative of the sugar-linked indolocarbazole family of anticancer natural products with demonstrated anti-kinase and anti-topoisomerase activity.

Taxanes are anti-mitotic, mitotic inhibitors or microtubule polymerization agents. Taxanes are characterized as compounds that promote assembly of microtubules by inhibiting tubulin depolymerization, thereby blocking cell cycle progression through centrosomal impairment, induction of abnormal spindles and suppression of spindle microtubule dynamics. Taxanes include but are not limited to, docetaxel and paclitaxel. The unique mechanism of action of taxane is in contrast to other microtubule poisons, such as Vinca alkaloids, colchicine, and cryptophycines, which inhibit tubulin polymerization. Microtubules are highly dynamic cellular polymers made of alpha-beta-tubulin and associated proteins that play key roles during mitosis by participating in the organization and function of the spindle, assuring the integrity of the segregated DNA. Therefore, they represent an effective target for cancer therapy.

Yet another embodiment of the present invention is the therapeutic combination or the therapeutic use in combination of a compound or composition of the present invention with TRAIL or other chemical or biological agents which bind to and activate the TRAIL receptor(s). TRAIL has received considerable attention recently because of the finding that many cancer cell types are sensitive to TRAIL-induced apoptosis, while most normal cells appear to be resistant to this action of TRAIL. TRAIL-resistant cells may arise by a variety of different mechanisms including loss of the receptor, presence of decoy receptors, or overexpression of FLIP which competes for zymogen caspase-8 binding during DISC formation. In TRAIL resistance, a compound or composition of the present invention may increase tumor cell sensitivity to TRAIL leading to enhanced cell death, the clinical correlations of which are expected to be increased apoptotic activity in TRAIL resistant tumors, improved clinical response, increased response duration, and ultimately, enhanced patient survival rate. In support of this, reduction in XIAP levels by in vitro antisense treatment has been shown to cause sensitization of resistant melanoma cells and renal carcinoma cells to TRAIL (Chawla-Sarkar, et al., 2004). The compounds of the present invention bind to IAPs and inhibit their interaction with caspases, therein potentiating TRAIL-induced apoptosis.

Compounds and compositions of the present invention also can be used to augment radiation therapy (or radiotherapy), i.e., the medical use of ionizing radiation as part of cancer treatment to control malignant cells. Although radiotherapy is often used as part of curative therapy, it is occasionally used as a palliative treatment, where cure is not possible and the aim is for symptomatic relief. Radiotherapy is commonly used for the treatment of tumors. It may be used as the primary therapy. It is also common to combine radiotherapy with surgery and/or chemotherapy. The most common tumors treated with radiotherapy are breast cancer, prostate cancer, rectal cancer, head & neck cancers, gynecological tumors, bladder cancer and lymphoma. Radiation therapy is commonly applied just to the localized area involved with the tumor. Often the radiation fields also include the draining lymph nodes. It is possible but uncommon to give radiotherapy to the whole body, or entire skin surface. Radiation therapy is usually given daily for up to 35-38 fractions (a daily dose is a fraction). These small frequent doses allow healthy cells time to grow back, repairing damage inflicted by the radiation. Three main divisions of radiotherapy are external beam radiotherapy or teletherapy, brachytherapy or sealed source radiotherapy and unsealed source radiotherapy, which are all suitable examples of treatment protocol in the present invention. The differences relate to the position of the radiation source; external is outside the body, while sealed and unsealed source radiotherapy has radioactive material delivered internally. Brachytherapy sealed sources are usually extracted later, while unsealed sources are injected into the body.

Administration of the compounds and compositions of the present invention may occur prior to, concurrently with, or subsequent to the combination treatment protocol. A variety of administration routes are available. The particular mode selected will depend, of course, upon the particular chemotherapeutic drug selected, the severity of the condition being treated and the dosage required for therapeutic efficacy. The methods of the invention, generally speaking, may be practiced using any mode of administration that is medically acceptable, meaning any mode that produces effective levels of the active compounds without causing clinically unacceptable adverse effects. Such modes of administration include, but are not limited to, oral, rectal, topical, nasal, intradermal, inhalation, intra-peritoneal, or parenteral routes. The term “parenteral” includes subcutaneous, intravenous, intramuscular, or infusion. Intravenous or intramuscular routes are particularly suitable for purposes of the present invention.

It is understood that the examples and embodiments described herein are for illustrative purposes only and that various modifications or changes in light thereof will be suggested to persons skilled in the art and are to be included within the spirit and purview of this application and the scope of the appended claims. For example, a further subset of compounds are those where R5 is hydroxy and R6 is H, in any of formulae (I), (II), (III) or (VIII) and in which either (1) both R3 and R4 are carbon atoms linked by a covalent bond or by an alkylene or alkenylene group of 1 to 8 carbon atoms where one to three carbon atoms can be replaced by O, S(O)ₙ or N(R8), or (2) R7 is selected from

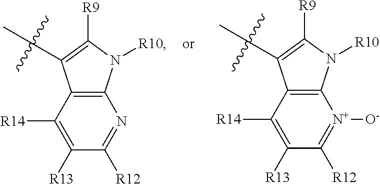

where R8 is H, hydroxy, alkoxy, aryloxy, alkyl, cycloalkyl, heterocycloalkyl, aryl, or heteroaryl and R9, R10, R12, R13 and R14 are independently selected from hydroxy, alkoxy, aryloxy, alkyl, or aryl.

1. A compound of Formula (I): 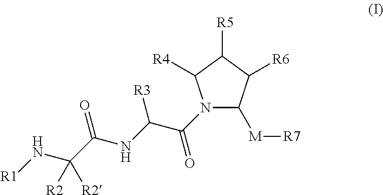
or a pharmaceutically acceptable salt thereof,
wherein:
R1 is H, hydroxy, alkyl, alkenyl, cycloalkyl, heterocycloalkyl, aryl, alkoxy, aryloxy, or heteroaryl;
R2 and R2′ are each independently H, alkyl, cycloalkyl, or heterocycloalkyl; or when R2′ is H then R2 and R1 can together form an aziridine or azetidine ring;
R3 and R4 are each independently H, alkyl, cycloalkyl, heterocycloalkyl, aryl, or heteroaryl; or, R3 and R4 are both carbon atoms linked by a covalent bond or by an alkylene or alkenylene group of 1 to 8 carbon atoms where one to three carbon atoms can be replaced by O, S(O)ₙ or N(R8);
R5 is H, hydroxy, alkoxy, aryloxy, alkyl, cycloalkyl, heterocycloalkyl, aryl, or heteroaryl;
R6 is H, hydroxy, alkoxy, aryloxy, alkyl, cycloalkyl, heterocycloalkyl, aryl, or heteroaryl,
R7 is alkyl, cycloalkyl, heterocycloalkyl, aryl, or heteroaryl;
R8 is H, hydroxy, alkoxy, aryloxy, alkyl, cycloalkyl, heterocycloalkyl, aryl, or heteroaryl;
M is a bond or an alkylene group of 1 to 5 carbon atoms;
n is 1 or 2, and
subject to the proviso that when R5 and R6 are both H, or when R5 is aryloxy and R6 is H, then either (1) R3 and R4 are both carbon atoms linked by a covalent bond or by an alkylene or alkenylene group of 1 to 8 carbon atoms where one to three carbon atoms can be replaced by O, S(O)ₙ or N(R8), or (2) R7 is selected from 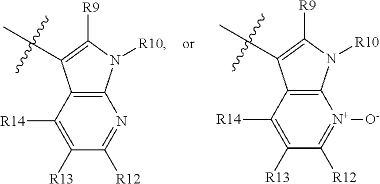
where R9, R10, R12, R13 and R14 are independently selected from hydroxy, alkoxy, aryloxy, alkyl, or aryl.

2. A compound of claim 1 having formula (II): 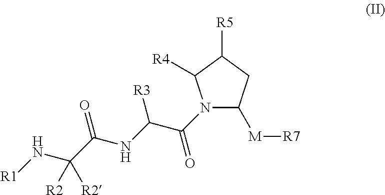
or a pharmaceutically acceptable salt thereof,
wherein:
R1 is H, hydroxy, alkyl, alkenyl, cycloalkyl, heterocycloalkyl, aryl, alkoxy, aryloxy, or heteroaryl;
R2 and R2′ are each independently H, alkyl, cycloalkyl, or heterocycloalkyl; or when R2′ is H then R2 and R1 can together form an aziridine or azetidine ring;
R3 and R4 are each independently H, alkyl, cycloalkyl, heterocycloalkyl, aryl, or heteroaryl; or, R3 and R4 are both carbon atoms linked by a covalent bond or by an alkylene or alkenylene group of 1 to 8 carbon atoms where one to three carbon atoms can be replaced by O, S(O)ₙ or N(R8);
R5 is H, hydroxy, alkoxy, aryloxy, alkyl, cycloalkyl, heterocycloalkyl, aryl, or heteroaryl;
R7 is alkyl, cycloalkyl, heterocycloalkyl, aryl, or heteroaryl;
R8 is H, hydroxy, alkoxy, aryloxy, alkyl, cycloalkyl, heterocycloalkyl, aryl, or heteroaryl;
M is a bond or an alkylene group of 1 to 5 carbon atoms;
n is 1 or 2, and
subject to the proviso that when R5 is H, or aryloxy, then either (1) R3 and R4 are both carbon atoms linked by a covalent bond or by an alkylene or alkenylene group of 1 to 8 carbon atoms where one to three carbon atoms can be replaced by O, S(O)ₙ or N(R8), or (2) R7 is selected from 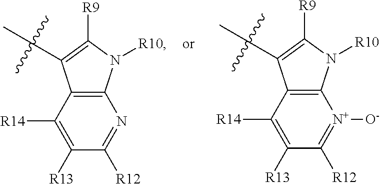
where R9, R10, R12, R13 and R14 are independently selected from hydroxy, alkoxy, aryloxy, alkyl, or aryl.

3. A compound or a pharmaceutically acceptable salt of claim 2 wherein R7 is selected from 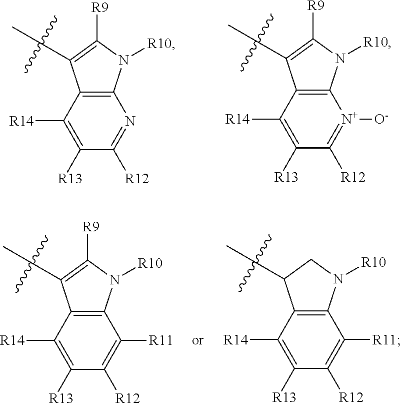
where R9, R10, R11, R12, R13 and R14 are independently selected from hydroxy, alkoxy, aryloxy, alkyl, or aryl.

4. A compound or a pharmaceutically acceptable salt of claim 3 wherein R7 is selected from

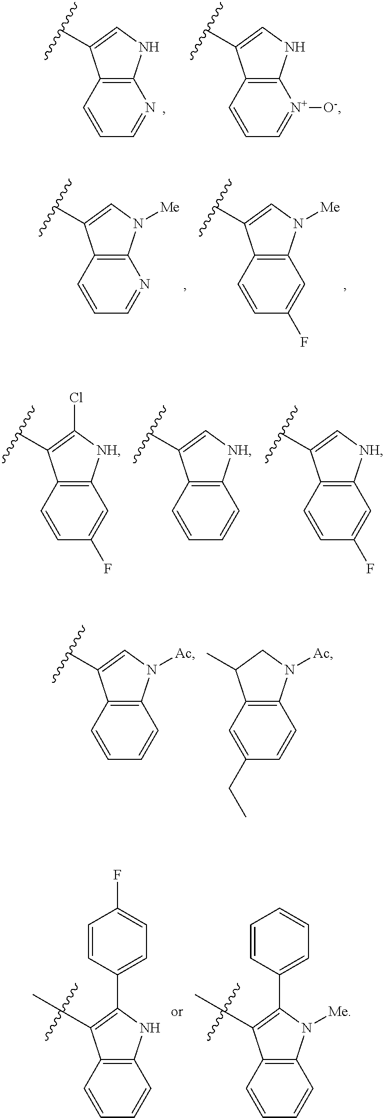

5. A compound or a pharmaceutically acceptable salt of claim 3 wherein R1 is methyl or ethyl; R2 is methyl, ethyl, or hydroxymethyl; R3 is isopropyl, tert-butyl, cyclohexyl, R-MeCHOMe, R-MeCHOH; R5 is H, or hydroxy; R6 is H, hydroxy, methyl, or methoxy.

6. A compound of claim 2 having the structure of formula (III): 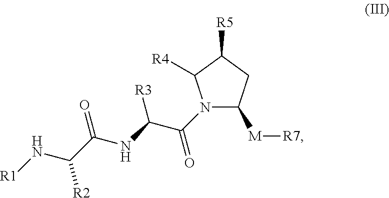
or a pharmaceutically acceptable salt thereof.

7. A compound of claim 1 having formula (IV): 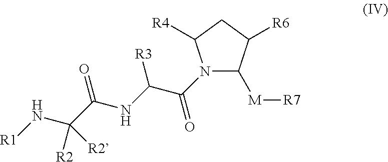
or a pharmaceutically acceptable salt thereof,
wherein:
R1 is H, hydroxy, alkyl, alkenyl, cycloalkyl, heterocycloalkyl, aryl, alkoxy, aryloxy, or heteroaryl;
R2 and R2′ are each independently H, alkyl, cycloalkyl, or heterocycloalkyl; or when R2′ is H then R2 and R1 can together form an aziridine or azetidine;
R3 and R4 are each independently H, alkyl, cycloalkyl, heterocycloalkyl, aryl, or heteroaryl; or, R3 and R4 are both carbon atoms linked by a covalent bond or by an alkylene or alkenylene group of 1 to 8 carbon atoms where one to three carbon atoms can be replaced by O, S(O)ₙ or N(R8),
R6 is hydroxy, alkoxy, aryloxy, alkyl, cycloalkyl, heterocycloalkyl, aryl, or heteroaryl;
R7 is alkyl, cycloalkyl, heterocycloalkyl, aryl, or heteroaryl;
R8 is H, hydroxy, alkoxy, aryloxy, alkyl, cycloalkyl, heterocycloalkyl, aryl, or heteroaryl;
M is a bond or an alkylene group of 1 to 5 carbon atoms; and
n is 1 or 2.

8. A compound or a pharmaceutically acceptable salt of claim 7 wherein R7 is selected from 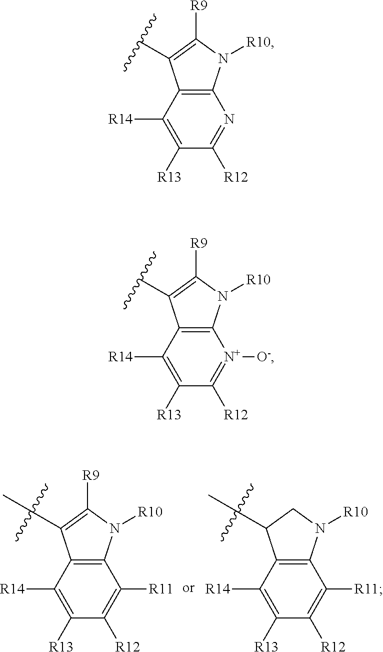
where R9, R10, R11, R12, R13 and R14 are independently selected from hydroxy, alkoxy, aryloxy, alkyl, or aryl.

9. A compound or a pharmaceutically acceptable salt of claim 8 wherein R7 is selected from

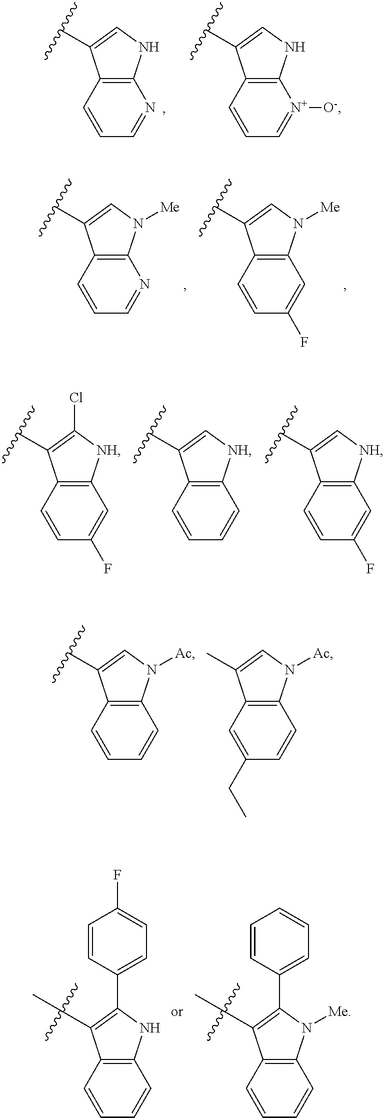

10. A compound or a pharmaceutically acceptable salt of claim 9 wherein R1 is methyl or ethyl; R2 is methyl, ethyl, or hydroxymethyl; R3 is isopropyl, tert-butyl, cyclohexyl, R-MeCHOMe, or R-MeCHOH; R5 is H, or hydroxy; R6 is H, hydroxy, methyl, or methoxy.

11. A compound of claim 7 having formula (V): 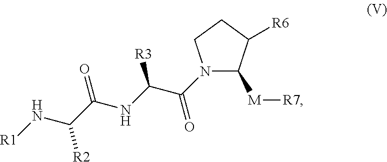
or a pharmaceutically acceptable salt thereof.

12. A compound of claim 11 having formula VI: 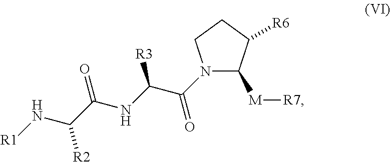
or a pharmaceutically acceptable salt thereof.

13. A compound of claim 11 having formula VII: 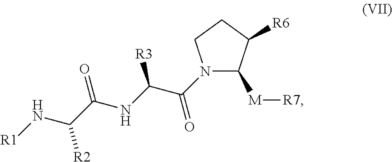
or a pharmaceutically acceptable salt thereof.

14. A compound of claim 1 having formula (VIII) 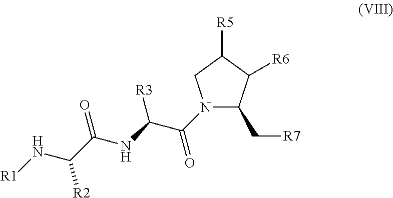
or a pharmaceutically acceptable salt thereof.

15. A compound of claim 1, or a pharmaceutically salt thereof, having the following formula and selected from the group consisting of compounds identified in the following table:

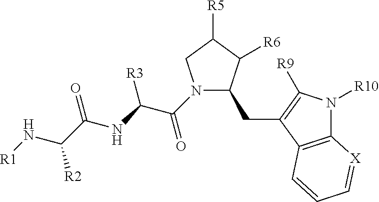

| Compound | R1 | R2 | R3 | R5 | R6 | R9 | R10 | X |
|---|---|---|---|---|---|---|---|---|
| A | Me | Me | R—MeCHOMe | (S)—OH | H | H | H | N |
| B | Me | Et | Cyclohexyl | (S)—OH | H | H | H | N |
| C | Me | Me | tert-Butyl | (S)—OH | H | H | H | N |
| D | Me | Me | R—MeCHOMe | H | H | H | H | N |
| E | Me | Me | tert-Butyl | H | H | H | H | N |
| F | Me | Et | R—MeCHOMe | H | H | H | H | N |
| G | Et | Et | R—MeCHOMe | H | H | H | H | N |
| H | Et | Me | R—MeCHOMe | H | H | H | H | N |
| I | Et | H | R—MeCHOMe | H | H | H | H | N |
| J | Me | CH₂OH | R—MeCHOMe | H | H | H | H | N |
| K | Et | Me | tert-Butyl | H | H | H | H | N |
| L | Me | Et | tert-Butyl | H | H | H | H | N |
| M | Et | Et | tert-Butyl | H | H | H | H | N |
| N | Et | H | tert-Butyl | H | H | H | H | N |
| O | Me | CH₂OH | tert-Butyl | H | H | H | H | N |
| P | Me | Me | R—MeCHOMe | H | H | H | H | N⁺—O⁻ |
| Q | Et | Me | R—MeCHOMe | H | H | H | H | N⁺—O⁻ |
| R | Et | Et | R—MeCHOMe | H | H | H | H | N⁺—O⁻ |
| S | Me | Me | tert-Butyl | H | H | H | H | N⁺—O⁻ |
| T | Me | Et | R—MeCHOMe | H | H | H | Me | N |
| U | Me | Me | R—MeCHOMe | H | H | H | Me | N |
| V | Me | Et | tert-Butyl | H | H | H | Me | N |
| W | Et | Me | tert-Butyl | H | H | H | Me | N |
| X | Me | Me | tert-Butyl | H | H | H | Me | N |
| Y | Me | Me | R—MeCHOMe | H | H | Ph | Me | N |
| Z | Me | Me | tert-Butyl | H | (S)—OH | H | H | N |
| AA | Et | Me | tert-Butyl | H | (S)—OH | H | H | N |
| BB | Me | Me | R—MeCHOMe | H | (S)—OH | H | H | N |
| CC | Et | Me | R—MeCHOMe | H | (S)—OH | H | H | N |
| DD | Me | Me | iPr | H | (S)—OH | H | H | N |
| EE | Et | Me | iPr | H | (S)—OH | H | H | N |
| FF | Me | Me | tert-Butyl | H | (S)—OMe | H | H | N |
| GG | Et | Me | tert-Butyl | H | (S)—OMe | H | H | N |
| HH | Et | Me | R—MeCHOMe | H | (S)—OMe | H | H | N |
| II | Et | Me | iPr | H | (S)—OMe | H | H | N |
| DD′ | Et | Me | R—MeCHOH | H | (R)—OH | H | H | CH |
| EE′ | Me | Et | R—MeCHOH | H | (R)—OH | H | H | CH |
| FF′ | Me | Me | R—MeCHOH | H | (R)—OH | H | H | CH |
| GG′ | Et | Me | R—MeCHOMe | H | (R)—OH | H | H | CH |
| HH′ | Me | Et | R—MeCHOMe | H | (R)—OH | H | H | CH |
| II′ | Me | Me | R—MeCHOMe | H | (R)—OH | H | H | CH |
| JJ′ | Et | Me | Cyclohexyl | H | (R)—OH | H | H | CH |
| KK′ | Me | Et | Cyclohexyl | H | (R)—OH | H | H | CH |
| LL′ | Me | Me | Cyclohexyl | H | (R)—OH | H | H | CH |
| MM′ | Me | cPr | tert-Butyl | H | (R)—OH | H | H | CH |
| NN′ | Me | Et | tert-Butyl | H | (R)—OH | H | H | CH |
| OO′ | Et | Me | tert-Butyl | H | (R)—OH | H | H | CH |
| PP′ | Me | Me | tert-Butyl | H | (R)—OH | H | H | CH |
| QQ′ | Me | cPr | cPr | H | (R)—OH | H | H | CH |
| RR′ | Me | Et | cPr | H | (R)—OH | H | H | CH |
| SS′ | Et | Me | cPr | H | (R)—OH | H | H | CH |
| TT′ | Me | cPr | iPr | H | (R)—OH | H | H | CH |
| UU′ | Me | Et | iPr | H | (R)—OH | H | H | CH |
| VV′ | Me | Me | cPr | H | (R)—OH | H | H | CH |
| WW′ | Et | Me | iPr | H | (R)—OH | H | H | CH |
| XX′ | Me | Me | iPr | H | (R)—OH | H | H | CH |
| YY′ | Me | Me | R—MeCHOMe | H | (R)—OMe | H | H | N |
| ZZ′ | Et | Me | iPr | H | (R)—OH | 4-F—Ph | H | CH |
| AAA | Me | Et | iPr | H | (R)—OH | 4-F—Ph | H | CH |
| BBB | Me | Me | iPr | H | (R)—OH | 4-F—Ph | H | CH |
| CCC | Et | Me | R—MeCHOH | H | (R)—OH | 4-F—Ph | H | CH |
| DDD | Me | Et | R—MeCHOH | H | (R)—OH | 4-F—Ph | H | CH |
| EEE | Me | Me | R—MeCHOH | H | (R)—OH | 4-F—Ph | H | CH |
| FFF | Et | Me | CH₂OMe | H | (R)—OH | 4-F—Ph | H | CH |
| GGG | Me | Et | CH₂OMe | H | (R)—OH | 4-F—Ph | H | CH |
| HHH | Me | Me | CH₂OMe | H | (R)—OH | 4-F—Ph | H | CH |
| III | Et | Me | Cyclohexyl | H | (R)—OH | 4-F—Ph | H | CH |
| JJJ | Me | Et | Cyclohexyl | H | (R)—OH | 4-F—Ph | H | CH |
| KKK | Me | Me | Cyclohexyl | H | (R)—OH | 4-F—Ph | H | CH |
| LLL | Et | Me | R—MeCHOMe | H | (R)—OH | 4-F—Ph | H | CH |
| MMM | Me | Et | R—MeCHOMe | H | (R)—OH | 4-F—Ph | H | CH |
| NNN | Me | Me | R—MeCHOMe | H | (R)—OH | 4-F—Ph | H | CH |

16. A compound of claim 1, or a pharmaceutically salt thereof, having the following formula where the stereochemistry at the carbon designated by * has an absolute (R) configuration and where the compound is selected from the group consisting of compounds identified in the following table:

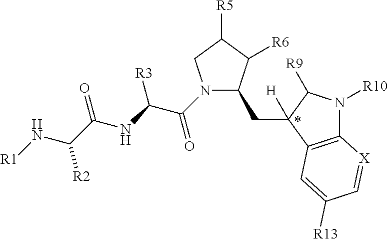

| Compound | R1 | R2 | R3 | R5 | R6 | R9 | R10 | X | R13 |
|---|---|---|---|---|---|---|---|---|---|
| AAAA | Et | Me | R—MeCHOMe | (S)—OH | H | H | Ac | CH | Et |
| BBBB | Me | Me | Cyclohexyl | (S)—OH | H | H | Ac | CH | Et |
| CCCC | Et | Me | Cyclohexyl | (S)—OH | H | H | Ac | CH | Et |
| DDDD | Me | Me | R—MeCHOMe | (S)—OH | H | H | Ac | CH | Et |
| EEEE | Et | Me | R—MeCHOMe | (S)—OH | H | H | Ac | CH | Et |
| VVV | Me | Me | Cyclohexyl | (S)—OH | H | H | Ac | CH | H |
| WWW | Me | Me | R—MeCHOMe | (S)—OH | H | H | Ac | CH | H |

17. A compound of claim 1, or a pharmaceutically salt thereof, having the following formula where the stereochemistry at the carbon designated by * has an absolute (5) configuration and where the compound is selected from the group consisting of compounds identified in the following table:

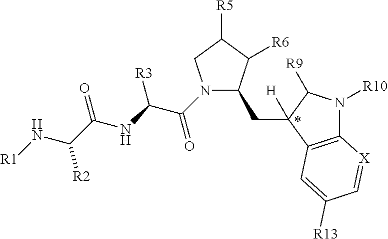

| Compound | R1 | R2 | R3 | R5 | R6 | R9 | R10 | X | R13 |
|---|---|---|---|---|---|---|---|---|---|
| XXX | Me | Me | Cyclohexyl | (S)—OH | H | H | Ac | CH | Et |
| YYY | Et | Me | Cyclohexyl | (S)—OH | H | H | Ac | CH | Et |
| ZZZ | Me | Me | R—MeCHOMe | (S)—OH | H | H | Ac | CH | Et |
| TTT | Me | Me | Cyclohexyl | (S)—OH | H | H | Ac | CH | H |
| UUU | Me | Me | R—MeCHOMe | (S)—OH | H | H | Ac | CH | H |

18. A compound of claim 1, or a pharmaceutically salt thereof, having the following formula and selected from the group consisting of compounds identified in the following table:

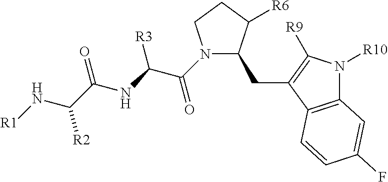

| Compound | R1 | R2 | R3 | R6 | R9 | R10 |
|---|---|---|---|---|---|---|
| JJ | Me | Me | R—MeCHOMe | (S)—Me | H | H |
| KK | Me | Et | R—MeCHOMe | (S)—Me | H | H |
| LL | Me | CH₂OH | R—MeCHOMe | (S)—Me | H | H |
| MM | Et | Me | R—MeCHOMe | (S)—Me | H | H |
| NN | Me | Me | R—MeCHOH | (S)—Me | H | H |
| OO | Me | Et | R—MeCHOH | (S)—Me | H | H |
| PP | Me | CH₂OH | R—MeCHOH | (S)—Me | H | H |
| QQ | Et | Me | R—MeCHOH | (S)—Me | H | H |
| RR | Me | Me | R—MeCHOMe | (S)—OH | H | H |
| SS | Et | Me | R—MeCHOMe | (S)—OH | H | H |
| TT | Me | Et | R—MeCHOMe | (S)—OH | H | H |
| UU | Me | Me | tert-Butyl | (S)—OH | H | H |
| VV | Me | Et | tert-Butyl | (S)—OH | H | H |
| WW | Me | Me | cyclo-Hexyl | (S)—OH | H | H |
| XX | Me | Et | cyclo-Hexyl | (S)—OH | H | H |
| YY | Me | Me | R—MeCHOMe | (S)—OH | H | Me |
| ZZ | Et | Me | R—MeCHOMe | (S)—OH | H | Me |
| A′ | Et | Me | R—MeCHOMe | (S)—OMe | H | Me |
| B′ | Me | Me | R—MeCHOMe | (S)—OMe | H | Me |
| C′ | Me | Et | R—MeCHOMe | (S)—OMe | H | Me |
| D′ | Me | Et | R—MeCHOMe | (S)—OMe | H | H |
| E′ | Me | Me | tert-Butyl | (S)—OMe | H | Me |
| F′ | Me | Et | tert-Butyl | (S)—OMe | H | Me |
| G′ | Et | Me | R—MeCHOMe | (S)—OMe | H | H |
| H′ | Me | Me | tert-Butyl | (S)—OMe | H | H |
| I′ | Et | Me | tert-Butyl | (S)—OMe | H | H |
| J′ | Me | Et | tert-Butyl | (S)—OMe | H | H |
| K′ | Et | Me | tert-Butyl | (S)—OMe | H | Me |
| L′ | Me | Me | R—MeCHOMe | (S)—OMe | H | H |
| M′ | Me | Me | R—MeCHOMe | (R)—OH | H | H |
| N′ | Me | Me | R—MeCHOH | (S)—OMe | Cl | H |
| O′ | Me | Et | R—MeCHOH | (S)—OMe | Cl | H |
| P′ | Me | Me | R—MeCHOMe | (S)—OMe | Cl | H |
| Q′ | Me | Et | R—MeCHOMe | (S)—OMe | Cl | H |
| R′ | Et | Me | R—MeCHOMe | (S)—OMe | Cl | H |
| S′ | Me | CH₂OH | R—MeCHOMe | (S)—OMe | Cl | H |
| T′ | Me | Me | iPr | (S)—OMe | Cl | H |
| U′ | Et | Me | iPr | (S)—OMe | Cl | H |
| V′ | Me | Et | iPr | (S)—OMe | Cl | H |
| W′ | Me | Me | cyclo-Hexyl | (S)—OH | Cl | H |
| X′ | Me | Et | tert-Butyl | (S)—OH | Cl | H |
| Y′ | Me | Me | tert-Butyl | (S)—OH | Cl | H |
| Z′ | Me | Et | iPr | (S)—OH | Cl | H |
| AA′ | Me | Me | iPr | (S)—OH | Cl | H |
| BB′ | Me | Et | R—MeCHOMe | (S)—OH | Cl | H |
| CC′ | Me | Me | R—MeCHOMe | (S)—OH | Cl | H |

19. A compound of claim 1, or a pharmaceutically salt thereof, having the following formula and selected from the group consisting of compounds identified in the following table:

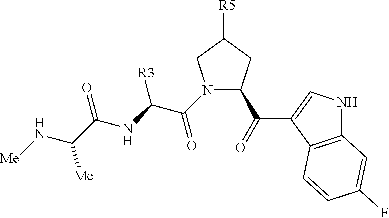

| Compound | R3 | R5 |
|---|---|---|
| OOO | cyclo-Hexyl | S—OH |
| PPP | tert-Butyl | S—OH |
| QQQ | iPr | S—OH |
| RRR | cyclo-Hexyl | R—OH |
| SSS | tert-Butyl | R—OH |

20. A pharmaceutical composition comprising a compound, or a pharmaceutically acceptable salt thereof, selected from claim 1 and a pharmaceutically acceptable excipient.

21. A method for inducing apoptosis in a cell comprising contacting the cell with a compound, or a pharmaceutically acceptable salt thereof, selected from claim 1 in an amount sufficient to induce apoptosis in the cell.

22. The method of claim 21 wherein the cell is a cancer cell.

23. A method of treating cancer selected from the group consisting of sarcomas, bladder cancers, ovarian cancers, breast cancers, brain cancers, pancreatic cancers, colon cancers, blood cancers, skin cancers, lung cancers and bone cancers, comprising administering a therapeutically effective amount of a compound, or a pharmaceutically acceptable salt thereof, selected from claim 1 to a patient in need thereof.

24. The method of claim 23 wherein the cancers are selected from colorectal cancer, renal carcinoma, ovarian carcinoma, pancreatic carcinoma, prostate carcinoma, breast carcinoma, melanoma, glioblastoma, acute myeloid leukemia (AML), small cell lung carcinoma, non-small cell lung carcinoma, rhabdomyosarcoma, and basal cell carcinoma.

25. The method of claim 23 further comprising administering a second therapy selected from radiation, chemotherapy, immunotherapy, photodynamic therapy and combinations thereof.

26. A method of treating an autoimmune disease selected from the group consisting of systemic lupus erythematosus, psoriasis and idiopathic thrombocytopenic purpura (Morbus Werlhof), comprising administering a therapeutically effective amount of a compound, or a pharmaceutically acceptable salt thereof, selected from claim 1 to a patient in need thereof.
